(19) United States
(12) Patent Application Publication
(10) Pub. No.: US 2026/0007692 A1
(43) Pub. Date: Jan. 8, 2026

(54) BIORESORBABLE ORGANIC BIOELECTRONICS

(71) Applicant: Roger Olsson, Malmö (SE)

(72) Inventor: Roger Olsson, Malmö (SE)

(21) Appl. No.: 18/875,811

(22) PCT Filed: Mar. 26, 2024
(86) PCT No.: PCT/SE2024/050275

§ 371 Date: Dec. 17, 2024

(30) Foreign Application Priority Data
Mar. 26, 2023 (SE) 2330136-9

(51) Int. Cl. A61K 31/795; A61K 31/381; C08F 134/04; C08K 5/45

(52) U.S. Cl. CPC A61K 31/795; A61K 31/381; C08F 134/04; C08K 5/45

(57) ABSTRACT

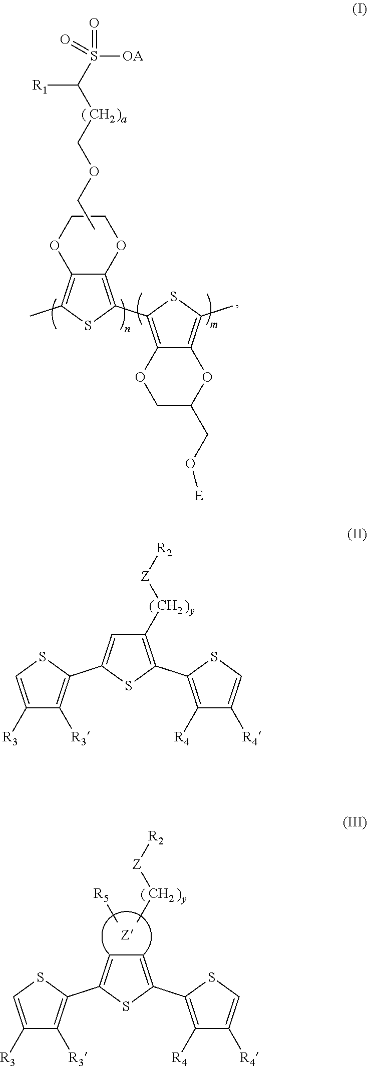

The present invention relates to a composition comprising a polymer of formula (I) and one or more compound of formula (II) or formula (III), and uses thereof in treating a disease, such as cancer, cardiovascular diseases, infections, or neurodegenerative diseases; wherein the polymer of formula (I) and the one or more compound of formula (II) or formula (III) are represented by the structures; and A, E, Z, Z′, R₁, R₂, R₃, R₃′, R₄, R₄′, R₅, R₆, R₇, R₈, R₉, R₁₀, n, m, a, y and q are as defined in the specification.

### TECHNICAL FIELD OF THE INVENTION

The present invention relates to a composition comprising a polymer of formula (I) and one or more of the compound, formula (II) or formula (III), and uses thereof in treating diseases, such as cancer, cardiovascular diseases, infections, immunomodulation, pain or neurodegenerative diseases, and energy storage.

### TECHNICAL BACKGROUND

Bioelectronics is a field of research in the junction of biology and electronics. Bioelectronic devices or bioelectronic medicine have the potential to complement classical therapies in nonchronic treatments, such as immunotherapy, pain, and cancer. Conventional drugs typically solely address biochemical processes, whereas bioelectronics tackles the dysfunction of bioelectric circuits. Further, prior art bioelectronic devices are facing problems with being invasive, needing surgical implantation methods, removal methods, or both, and primarily focusing on chronic illnesses. These types of devices typically face the need for surgical removal after disease relief. Various strategies have been undertaken to design electrodes that seamlessly connect to the structures of the nervous system in animals, where most of these depend on external cues, such as electric fields, chemicals including proteins, or genetic engineering. For example, pioneering work presented in the prior art focus on conducting polymers that forms within the brains of rodents, i.e., the in vivo electropolymerization of 3,4-ethylenedioxythiophene (EDOT) monomer templated on poly(styrene sulfonate) (PSS), thus forming poly(3,4-ethylenedioxythiophene) (PEDOT):PSS structures in the brain of a mouse. However, the formed polymer mixture protruded from the injection electrode as a massive cloud without succeeding in cellular integration.

In view of the above, improved bioelectronics is needed that may offer minimally invasive approaches and new ways to treat diseases, especially within the brain, which may avoid the limitations of traditional open-skull surgery or rigid electrode implantation.

### SUMMARY OF THE INVENTION

In view of the above, it is an object of the present technology to provide self-organizing substrate-free organic electrodes that have the potential to meet the above-mentioned drawbacks and the criteria and the need for seamless integration into dynamic biological systems in a way that is not possible for classical rigid solid-state electronics.

Another object is to provide a general approach to generating bioelectronics that is not dependent on specific external or endogenous triggers.

Another object is to provide a composition or a pharmaceutical composition that is biocompatible, i.e., well tolerated in the human body.

Another object is to provide a composition or pharmaceutical composition that comprises electrically conductive structures that may be placed in the targeted area at a precise localization.

Another object is to provide a composition that may assemble to bioresorbable electrodes from e.g., from water-dispersed nanoparticles in the brain, in and around a tumor, cardiac tissue, and on and around the vascular system.

Another object is to provide a composition that, in situ, may function as electrodes, and that might set off a controlled cellular response in a target organ, e.g., brain, heart, tumor, spinal cord, when relaying external stimuli.

Another object is to provide electroactive organic polymers that may be generated with spatial control in vivo using different modalities such as enzymatic, photochemically, i.e., light-induced, electrochemically controlled processes, or combinations thereof.

Another object of the present disclosure is to provide a composition that does not cause tissue damage or cell and organ toxicity after bioresorption.

Another object is to provide a composition or a pharmaceutical composition that may be in vivo assembled, fully integrated, and transient organic bioelectronics within cellular structures that may be used for nonchronic treatments.

Another object is to provide a composition that is easy and cheap to synthesize from commercially available and affordable materials.

Thus, to achieve at least one of the above objects and also other objects that will be evident from the following description, a composition or a pharmaceutically acceptable salt thereof, a pharmaceutical composition, or a kit, for use in the treatment or prophylaxis of a disease, such as cancer, cardiovascular diseases, infections, or neurodegenerative diseases, and a use of a composition or a pharmaceutically acceptable salt thereof and a method of treating cancer, cardiovascular disease, infections, or neurodegenerative diseases are as defined in the independent claims. Preferred variations to the inventive concept will be evident from the dependent claims.

According to a first aspect, there is provided a composition comprising a polymer of formula (I) and one or more compound of formula (II) or formula (III), wherein the polymer of formula (I) is represented by the structure:

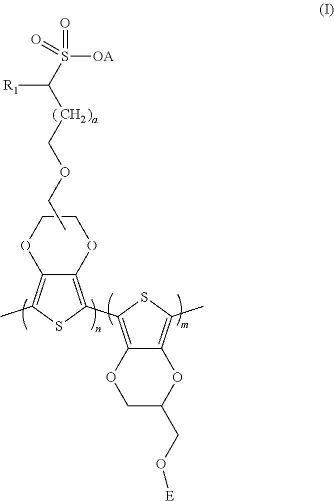

the one or more compound of formula (II) or formula (III) is represented by the structures:

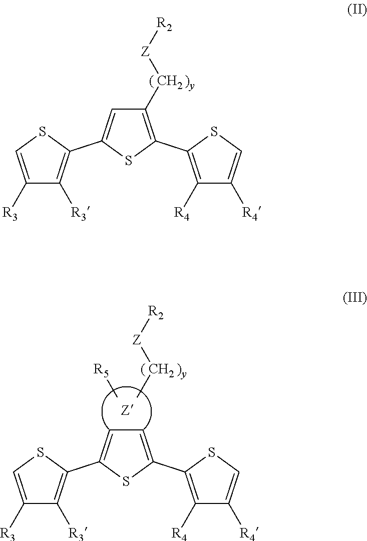

and wherein each A is selected from H, Na, K, Li, Ca, Mg, Sr, and Ba; E is selected from H, C₁₋₆ alkyl, C₂₋₆ alkenyl, C₂₋₆ alkynyl, C₃₋₆ cycloalkyl, aryl, heteroaryl, —(CH₂CH₂O)_{q}CH₂CH₂OH, wherein said C₁₋₆ alkyl is optionally substituted with one or more of —N₃, —OH, —SO₃A, —N(C₁₋₆ alkyl)₂, —NH⁺(C₁₋₆ alkyl)₂, and —N⁺(C₁₋₆ alkyl)₃; R₁ is selected from H, C₁₋₆ alkyl, C₂₋₆ alkenyl, C₂₋₆ alkynyl, C₃₋₆ cycloalkyl, aryl, heteroaryl, wherein said C₁₋₆ alkyl is optionally substituted with one or more —N(C₁₋₆ alkyl)₂, —NH⁺(C₁₋₆ alkyl)₂, and —N⁺(C₁₋₆ alkyl)₃; each Z is selected from a bond, —O—, —OP(O)(O⁻)O—, —OP(O)(OH)O—, —OC(O)—, —C(O)O—, —OC(O)NH—; each R₂ is selected from H, C₁₋₂₀ alkyl, aryl, heterocyclyl, heteroaryl, and Si(C₁₋₆ alkyl)₃, said C₁₋₂₀ alkyl, aryl, heterocyclyl and heteroaryl is optionally substituted with one or more R₅ or R₆; each of R₃, R₃′, R₄ and R₄′ is selected from H and —(CH₂)_{y}—Z—(R₂), when R₃ and R₃′ are C₁₋₆ alkoxy and taken together with the atom to which they are each attached form a heterocycle optionally substituted with one or more R₅ or R₂; when R₄ and R₄′ are C₁₋₆ alkoxy and taken together with the atom to which they are each attached form a heterocycle optionally substituted with one or more R₅ or R₂; R₅ is selected from H, C₁₋₆ alkyl, C₂₋₆ alkenyl, and C₂₋₆ alkynyl; Z′ is 6- or 7-membered heterocycle; each R₆ is selected from-SO₃A, —CO₂A, —CO₂(R₉), —OH, —O(R₉), halogen, —N₃, —NH₂, —NH(R₉), —NHC(O)(R₉), —N(C₁₋₆ alkyl)₂, —N⁺(C₁₋₆ alkyl)₃, —(OCH₂CH₂)_{q}—(R₈), C₁₋₆ alkyl, C₂₋₆ alkenyl, C₂₋₆ alkynyl, aryl, heteroaryl, heterocyclyl, ferrocenyl, —C(O)NH(C₁₋₆ alkyl), wherein said —N⁺(C₁₋₆ alkyl)₃ and —C(O)NH(C₁₋₆ alkyl) being optionally substituted with one or more R₇, and said C₁₋₆ alkyl, heterocyclyl, heteroaryl, and aryl being substituted with one or more R₇, R₉ or R₁₀; R₇ is selected from-SO₃A, —CO₂A, —(OCH₂CH₂)_{q}—(R₈), —NH₂, —NHC(O)—(R₉), —OC(O)—(R₉), aryl, guanidinyl, —C(O)NH(C₁₋₆ alkyl), wherein said guanidinyl and —C(O)NH(C₁₋₆ alkyl) being optionally substituted with one or more R₈; R₈ is selected from C₁₋₆ alkyl, C₁₋₆ alkoxy, —OH, —NH₂, —NH(C₁₋₆ alkyl), —N₃, —OC(O)—(R₉), and —C(O)NH(C₁₋₆ alkyl), wherein said C₁₋₆ alkyl and —C(O)NH(C₁₋₆ alkyl) being optionally substituted with one or more R₉; R₉ is selected from C₁₋₆ alkyl, C₂₋₆ alkenyl, C₂₋₆ alkynyl, aryl, heteroaryl, heterocyclyl, ferrocenyl, —B(OH)₂, —CO₂A, and —CO₂(C₁₋₆ alkyl), wherein said C₁₋₆ alkyl, aryl, heterocyclyl and —CO₂(C₁₋₆ alkyl) being optionally substituted with one or more of R₁₀; R₁₀ is selected from oxo, —SO₃A, —NH₂, —CO₂A, —OH, —P(O)(OH)₂, C₁₋₆ alkyl, C₁₋₆ alkoxy, aryl, and heteroaryl; and n is 4-32; m is 0-10; a is 1-5; y is 0-16; q is 0-15; or a pharmaceutically acceptable salt thereof.

According to another aspect there is provided a composition of the first aspect, or a pharmaceutically acceptable salt thereof, for use in the treatment or prophylaxis of a disease.

According to another aspect there is provided a composition of the first aspect, or a pharmaceutically acceptable salt thereof, for use in the treatment or prophylaxis of a disease, said disease being selected from cancer, cardiovascular diseases, infections, and neurodegenerative diseases.

According to another aspect there is provided a use of a composition of the first aspect, or a pharmaceutically acceptable salt thereof, in the preparation of a medicament for treating cancer, cardiovascular diseases, infections, or neurodegenerative diseases.

According to another aspect there is provided a use of a composition of the first aspect, or a pharmaceutically acceptable salt thereof, in the preparation of a medicament for treating spinal cord trauma, such as spinal cord injuries, wherein said medicament induces regeneration of nerves in spinal cord.

According to another aspect there is provided a method of treating cancer, cardiovascular diseases, infections, or neurodegenerative diseases, comprising administering a therapeutically effective amount of a composition according to the first aspect, or a pharmaceutically acceptable salt thereof, to a patient in need thereof.

According to another aspect there is provided a pharmaceutical composition comprising a composition according to the first aspect, or a pharmaceutically acceptable salt thereof, and a pharmaceutically acceptable diluent, carrier and/or excipient.

According to another aspect there is provided a pharmaceutical composition, comprising a therapeutically effective amount of a composition according to the first aspect, or a pharmaceutically acceptable salt thereof, and another anticancer agent selected from alkylating agents, antimetabolites, anticancer camptothecin derivatives, plant-derived anticancer agents, antibiotics, enzymes, platinum coordination complexes, tyrosine kinase inhibitors, hormones, hormone antagonists, monoclonal antibodies, interferons, and biological response modifiers.

According to another aspect there is provided a hydrogel comprising a composition according to the first aspect, for use in the treatment or prophylaxis of a disease.

According to another aspect there is provided a kit comprising a composition according to the first aspect, for use in the treatment or prophylaxis of a disease.

### DETAILED DESCRIPTION OF THE INVENTION

The present inventive concept is focused on a composition that finds uses in a novel modality of intratumor electrotherapy that incorporates redox processes, ionic currents, or a combination of both. The present invention relates to compositions that may self-assemble into transient conductive electrodes that are biocompatible and bioresorbable. The fluidic nature of the conductive structure suggests that it can be implanted using minimally invasive techniques and seamlessly integrated into and around the tumor. Moreover, the ability to modify electrode properties post-implantation facilitates targeting more precise mechanisms related to bioelectrical circuitry dysfunctions in cancer. This soft electrode may be employed as a stand-alone treatment or in conjunction with irreversible electroporation (IRE) technology, such as Nanoknife. In the latter scenario, the tumor-integrated soft electrode will help the Nanoknife electrodes deliver the therapy throughout the entire tumor. Thus, the present disclosure is focused on a general approach that is not dependent on specific external or endogenous triggers to assemble compositions into bioresorbable high-performing electrode structures within e.g., the central nervous system (CNS) implanted using a minimally invasive method.

Objects within the body, such as nerves and tissues, can greatly differ in size and shape, impacting how medical devices like electrodes interact with them. Nerve bundles and ganglia, for instance, can have highly irregular shapes and sizes, making standard, one-size-fits-all electrodes problematic due to fit and adaptability issues. Traditional electrodes often require invasive surgical procedures for implantation, including making large incisions to provide direct access to the target area. This process can lead to significant trauma, bleeding, and an inflammatory response, which may result in the growth of connective tissue that can interfere with the electrode's function. Moreover, the shapes of conventional electrodes are frequently determined by manufacturing processes, limiting their flexibility to accommodate the diverse anatomical features found within the central and peripheral nervous systems. For example, flat electrodes produced using silicon wafer techniques or rod-shaped electrodes designed for deep brain stimulation can create non-uniform and imprecise electrical fields, posing challenges in effectively stimulating the intended targets without affecting adjacent areas. Additionally, the surgical placement of these electrodes can provoke further tissue irritation and inflammation over time, potentially diminishing the electrode's effectiveness and requiring additional medical interventions. There's also the concern of the financial and health risks associated with surgical implantation procedures, which can deter patients from opting for these treatments. Given these challenges, there's a clear need for an innovative electrode design that can be introduced into the body in a less invasive manner, such as through injection. Ideally, this new type of electrode would mold to and encapsulate the targeted nerve or tissue, creating a more effective and uniform electrical field, minimizing trauma, and providing more stable and long-term outcomes.

The present inventive concept will now be described more fully hereinafter with reference to the accompanying schemes and drawings, showing preferred variants of the inventive concept.

The present disclosure relates to a composition, or a pharmaceutically acceptable salt thereof, comprising a polymer of formula (I) and one or more compound of formula (II) or formula (III), wherein the polymer of formula (I) and the one or more compound of formula (II) or formula (III) are represented by the structures:

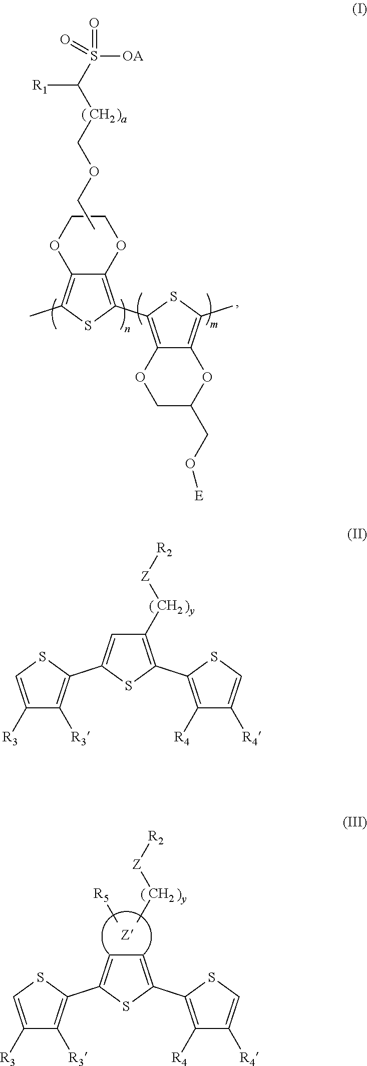

wherein A, E, Z, Z′, R₁, R₂, R₃, R₃′, R₄, R₄′, R₅, R₆, R₇, R₈, R₉, R₁₀, n, m, a, y and q are as described above.

In the conceptualization of the material design, it was anticipated that upon being injected into the tissue, the polymer of formula (I) would induce self-aggregation and the formation of a conductive framework. Subsequently, one or more trimers of formula (II) or formula (III) in the mixture, being smaller than the polymer backbone, will diffuse within and out from the backbone. Applying an external stimuli, such as a low electrical potential, light or enzyme, functionalize the full volume of the polymer backbone with trimers attaching to and extending out from it and thereby changing the properties of the structure. This method positions the conductive framework precisely at the targeted site and facilitates more extensive diffusion into the surrounding tissue and cells. It allows control over the chemical properties since trimers with different moieties can be used during functionalization.

This inventive concept may, however, be embodied in many different forms and should not be construed as limited to the variants set forth herein; rather, these variants are provided for thoroughness and completeness, and fully convey the scope of the present disclosure to the skilled person. The details of the present disclosure are set forth in the accompanying description below. Although methods and materials similar or equivalent to those described herein can be used in the practice or testing of the present disclosure, illustrative methods and materials are now described. Other features, objects, and advantages of the present disclosure will be apparent from the description and from the claims. In the description and the appended claims, the singular forms also include the plural unless the context clearly dictates otherwise. Unless defined otherwise, all technical and scientific terms used herein have the same meaning as commonly understood by one of ordinary skill in the art to which this invention belongs.

Although individual features may be included in different variants, these may possibly be combined in other ways, and the inclusion in different variants does not imply that a combination of features is not feasible. In the context of the present disclosure, the terms “a”, “an” does not preclude a plurality. The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups but does not necessarily have any further functional groups. Suitable substituents used in the optional substitution of the described groups are further defined and described below.

In the polymer of formula (I), the number of repeating units is annotated with n and m, wherein n is between 4 and 32, preferably between 5 and 12, and m is between 0 and 10, preferable between 0 and 3. In an embodiment, m is 0 or 1, and E is then preferably selected from H, C₁₋₆ alkyl, —(CH₂CH₂O)_{q}CH₂CH₂OH, C₁₋₆ alkyl substituted with —N₃, C₁₋₆ alkyl substituted with —SO₃A, and C₁₋₆ alkyl substituted with-SO₃A and —N⁺(C₁₋₆ alkyl)₃, and R₁ is preferably selected from H, C₁₋₆ alkyl, such as methyl, and C₁₋₆ alkyl substituted with —N⁺(C₁₋₆ alkyl)₃. When m is 1, E is preferably H, and R₁ is preferably selected from H and C₁₋₆ alkyl, such as methyl. When m is 0, R₁ is preferably selected from H and C₁₋₆ alkyl substituted with —N⁺(C₁₋₆ alkyl)₃.

In an embodiment, the one or more compound of formula (II) or formula (III) are one or more compounds of formula (II-a), formula (III-a), formula (III-b), formula (III-c), and formula (III-d), and the composition thereby comprises the polymer of formula (I) and the one or more compounds of formula (II-a), formula (III-a), formula (III-b), formula (III-c), and formula (III-d). The compounds of formula (II-a), (III-a), (III-b), (III-c), and (III-d) are represented by the structures:

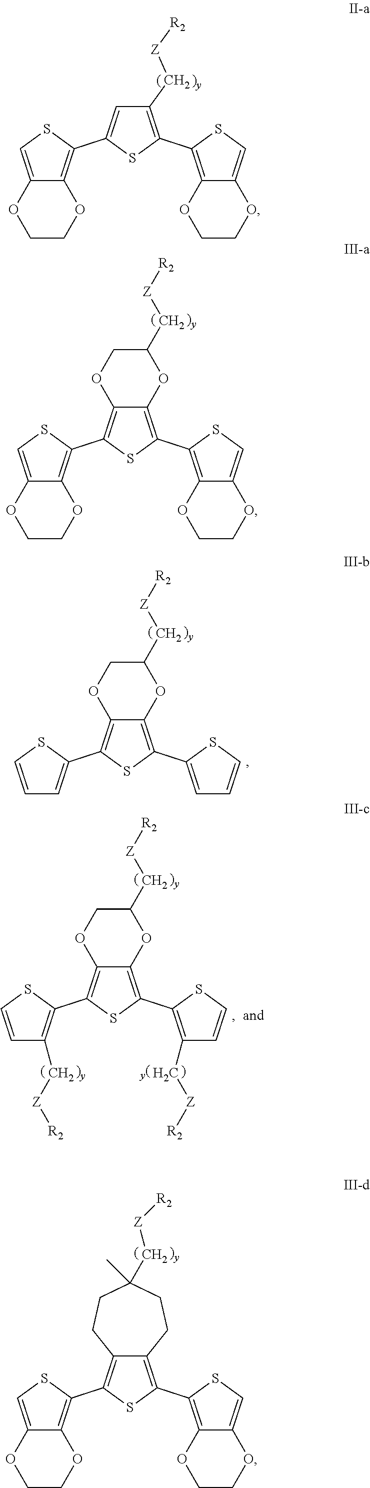

or a pharmaceutically acceptable salt thereof, wherein Z, R₂, R₃, R₃′, R₄, R₄′, R₅, R₆, R₇, R₈, R₉, R₁₀, y and q are as described above. The composition thus comprises the co-polymer of formula (I) and the one or more compounds of formula (II-a), formula (III-a), formula (III-b), formula (III-c), and formula (III-d).

Substituent R₂ in any one of the previously-mentioned structures may be selected from the following:

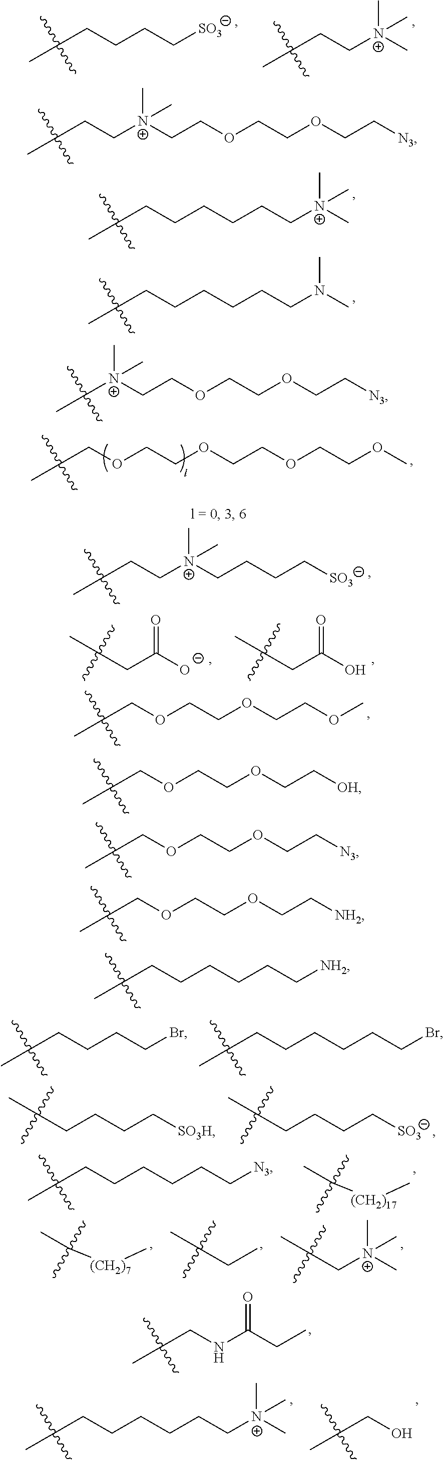

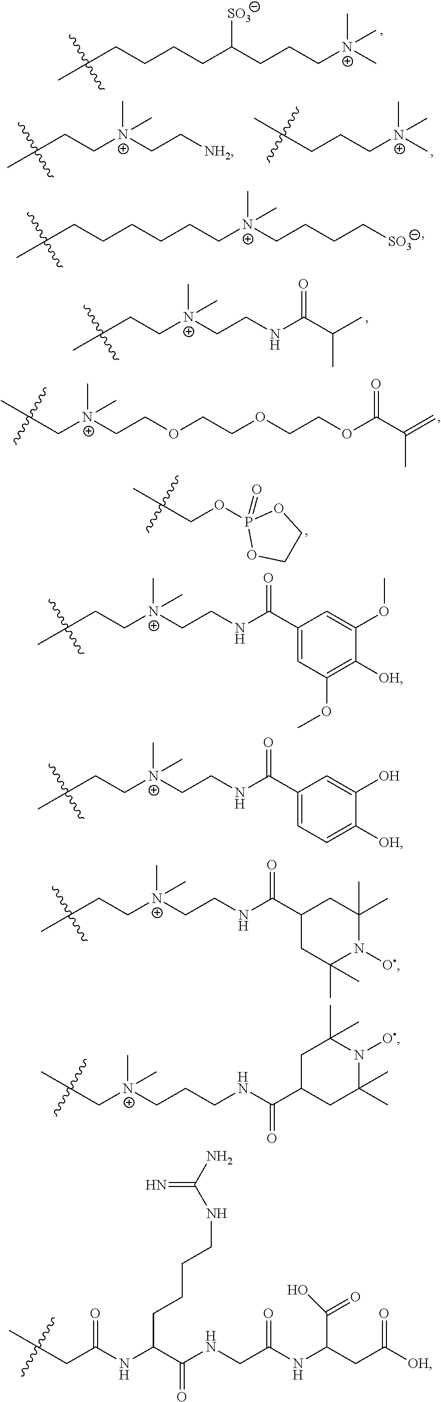

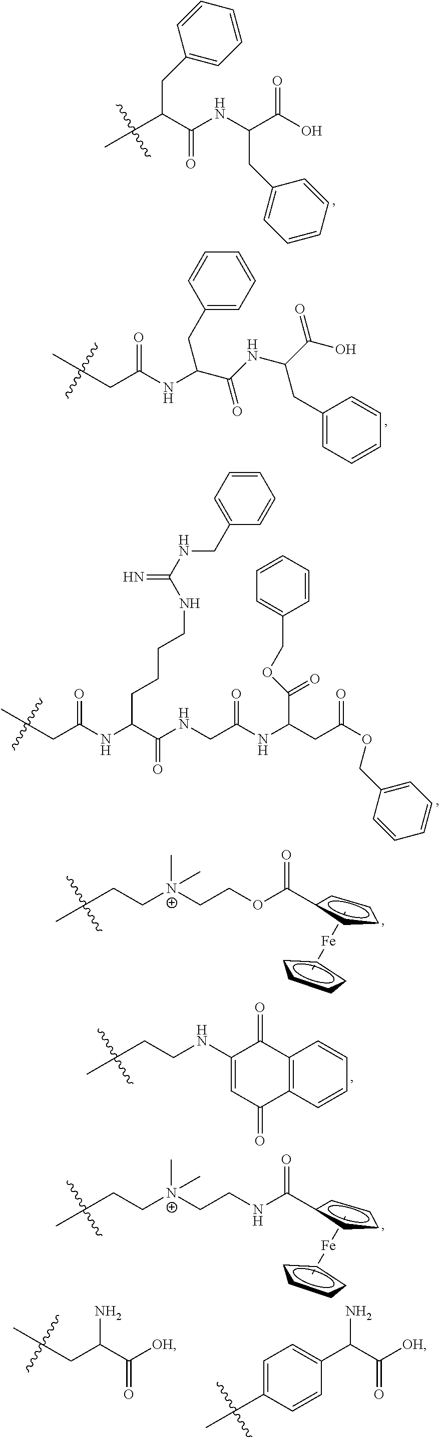

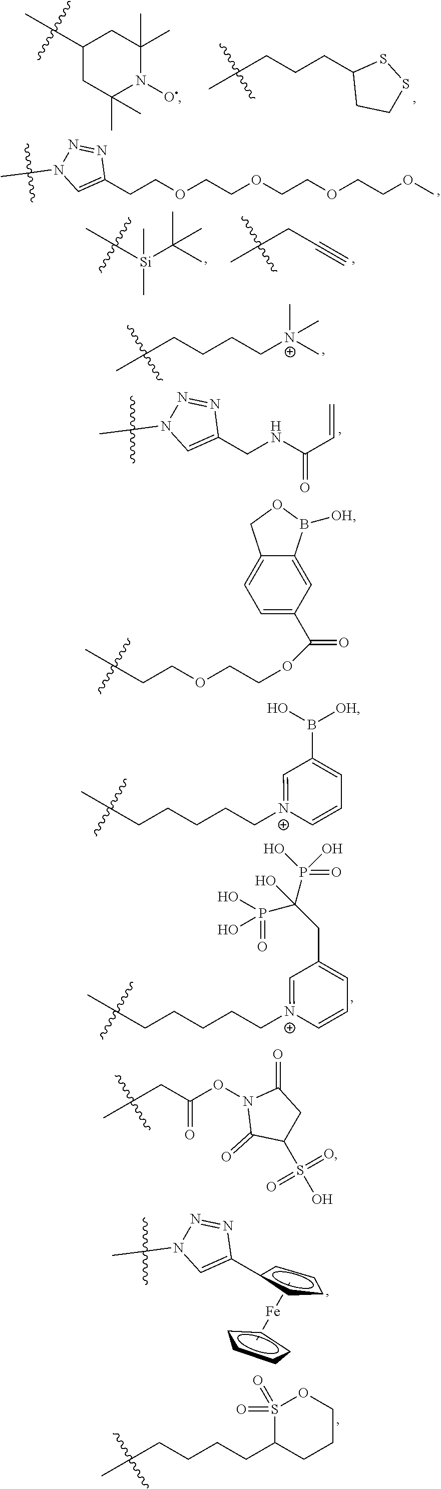

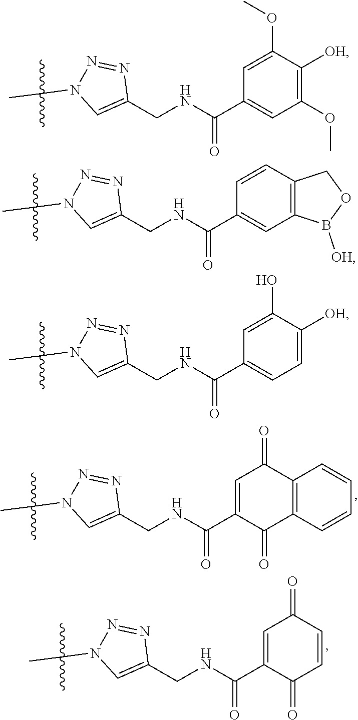

—N₃, —Cl, or a pharmaceutically acceptable salt thereof.

In an embodiment, the composition of the present disclosure comprises the co-polymer represented by the structure (I-1)

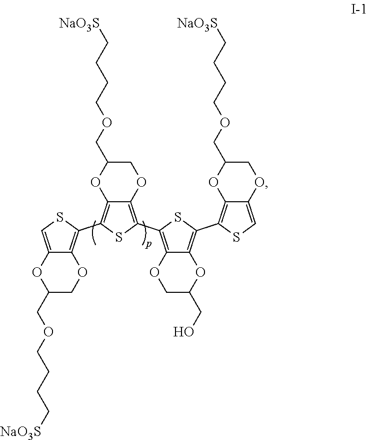

wherein p is 1-12; and the one or more compound represented by the structures (II-1) and (II-2)

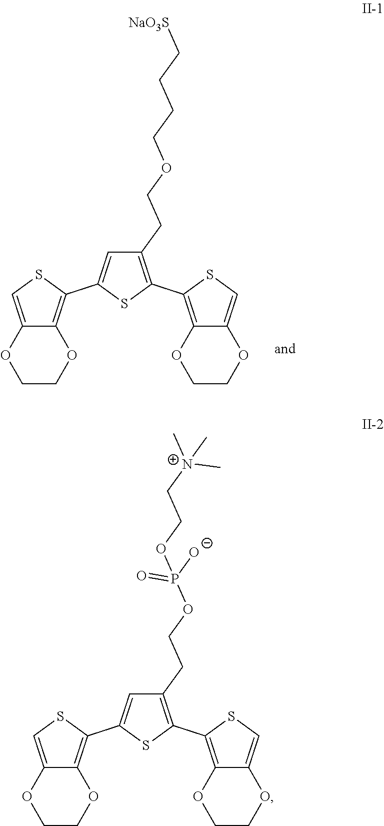

or a pharmaceutically acceptable salt thereof.

The composition comprising a polymer of formula (I) and one or more compounds of formula (II) or formula (III), or a pharmaceutically acceptable salt thereof, is used in the treatment or prophylaxis of a disease, such as cancer, cardiovascular diseases, infections, and neurodegenerative diseases. Alternatively, there is provided a method of treating cancer, cardiovascular diseases, infections, or neurodegenerative diseases, comprising administering a therapeutically effective amount of the composition, or a pharmaceutically acceptable salt thereof, to a patient in need thereof.

A significant benefit of this new invention is its ability to be administered directly to the target area without the need for traditional surgical methods such as cutting through tissue with scalpels or scissors. This method greatly reduces or eliminates damage to the targeted and surrounding areas. The invention uniquely adjusts to the precise contours of the target area, effectively creating a custom fit by forming around the target's shape. This is achieved through a versatile system that utilizes a combination of external forces (such as electronic and light energies) and internal processes (like enzyme reactions) to mold and adapt to the target. This approach ensures an optimal electrical connection and secures the device in place through enhanced mechanical bonding to the target, offering a stable and effective solution. Moreover, through injection the cured electrode of the present disclosure may be placed in hard to reach locations in the body which a surgeon might be unwilling to place a prior art device with elective general surgery, e.g., ganglia of the sympathetic chain or nerves of the CNS or PNS adjacent to major blood vessels and located medially in the body which are difficult to access on a direct line from outside of the body. The present invention introduces a novel bioelectronic approach to cancer therapy by leveraging a composition according to the first aspect that includes compounds from formulas (I), (II), and/or (III). This composition may be used to target cancer cells directly, disrupting their growth and proliferation. By integrating this polymer into bioelectronic devices, it's possible to deliver targeted treatments that disrupt cancerous activities at the molecular level, offering a new avenue for combating various types of cancer with potentially fewer side effects than traditional therapies. Typically, said cancer is selected from brain cancer, glioblastoma, neuroblastoma, prostate cancer, breast cancer, and solid tumors.

For neurodegenerative diseases, the composition according to the first aspect can target and modulate neuronal functions and degeneration pathways. By integrating this composition into bioelectronic systems, it is possible to influence cellular processes involved in diseases like Alzheimer's and Parkinson's, potentially slowing disease progression or alleviating symptoms through targeted electronic interventions. Typically, said neurogenerative diseases is selected from brain trauma, spinal cord trauma, trauma to the peripheral nervous system, and motor neuron disease.

For cardiovascular diseases, the composition according to the first aspect can interact with biological processes that underlie heart and vascular conditions. By incorporating this composition into bioelectronic systems, the technology can offer precise control over heart rhythms, blood flow, and vascular health, potentially treating or managing conditions such as arrhythmias, hypertension, and atherosclerosis. This method represents a groundbreaking step in the use of bioelectronics for cardiovascular interventions. Typically, said cardiovascular diseases are selected from coronary artery diseases (e.g. angina, heart attack), heart failure, hypertensive heart disease, rheumatic heart disease, cardiomyopathy, arrhythmia, congenital heart disease, valvular heart disease, carditis, aortic aneurysms, peripheral artery disease, thromboembolic disease, and venous thrombosis.

The composition according to the first aspect also finds application in controlling infections. By targeting the bioelectronic interactions within pathogens or the infected host's cells, the composition can inhibit the replication of viruses or bacteria, or modulate the body's immune response to these pathogens. This approach opens new doors for treating infections, especially those resistant to conventional drugs, through bioelectronic means. Typically, said infections are selected from viral infections, bacterial infections, parasitic infections and fungal infections.

In the context of immunomodulation, the composition according to the first aspect can be used to modulate the immune system's activity. This bioelectronic application has the potential to treat autoimmune diseases, reduce inflammation, or enhance the immune response against pathogens and tumors. By adjusting the immune system's electronic signals, this technology offers a novel pathway to influence immune-related diseases and conditions. Thus, a composition according to the first aspect may be used to modulate the immune system's activity as described above.

Further, the composition according to the first aspect can be used in the application of pain management, that involves using the composition within bioelectronic devices to target nerve signals that convey pain. This method provides a new strategy for managing chronic pain conditions by interrupting or modulating pain signals before they reach the brain, offering a potentially effective and non-pharmacological alternative to traditional pain treatments. Thus, a composition according to the first aspect may be used in pain management as described above.

The composition, or a pharmaceutically acceptable salt thereof, can be used in the preparation of a medicament for treating cancer, cardiovascular diseases, infections, or neurodegenerative diseases, such as a medicament for treating cancer, cardiovascular diseases, infections, or neurodegenerative diseases. Typically, said cancer is selected from brain cancer, glioblastoma, neuroblastoma, prostate cancer, breast cancer, and solid tumors, and said neurogenerative diseases is selected from brain trauma, spinal cord trauma, trauma to the peripheral nervous system, and motor neuron disease.

The composition, or a pharmaceutically acceptable salt thereof, can also be used in the preparation of a medicament for treating spinal cord trauma, such as spinal cord injuries, wherein said medicament induces regeneration of nerves in the spinal cord.

Furthermore, a pharmaceutical composition comprising the composition, or a pharmaceutically acceptable salt thereof, and a pharmaceutically acceptable diluent, carrier and/or excipient is provided. The pharmaceutical composition can also comprise another anticancer agent selected from alkylating agents, antimetabolites, anticancer camptothecin derivatives, plant-derived anticancer agents, antibiotics, enzymes, platinum coordination complexes, tyrosine kinase inhibitors, hormones, hormone antagonists, monoclonal antibodies, interferons, and biological response modifiers.

Alternatively, a hydrogel or a kit comprising the composition, or a pharmaceutically acceptable salt thereof, is provided, and said hydrogel or said kit can be used in the treatment or prophylaxis of a disease. The kit may have different parts or compartments, such that the compound of formula (I), or a pharmaceutically acceptable salt thereof, is in a first part of the kit, and the one or more compound of formula (II) or formula (III), or a pharmaceutically acceptable salt thereof, is in a second part of the kit.

The particular mechanical and structural properties of the the formed electrode might be varied to match the properties of the tissue targeted, by the selection of the conductive elements. The formation process, i.e., by introducing electrical energi, light, enzymatic reactions, or combinations there of. For example, The Young's modulus measured of BICS (32±6 kPa) closely matched the agarose (38±5 kPa). This is orders of magnitude lower than what has been reported for PEDOT:PSS hydrogels (2-20 MPa) and is within the range of human myocadiac tissue (10-200 kPa). The increase in elastic modulus compared to our previously reported injectable bioresorbable electrodes for brain tissue—that had a shear modulus of 0.57±0.1 kPa compared to 0.5-1 kPa for brain tissue—corroborates the additional stickiness needed for the dynamic heart tissue compared to brain tissue and the materials match better with respective tissue.

Additionally, the present disclosure is put into place without the far greater costs of general surgery, and the attendant risks from general anesthesia and infection. The present invention may be placed by pain physicians accustomed to the placement of pharmacological nerve blocks with or without the aid of ultrasound or angiography as means for visualization.

The present invention also has another distinct advantage over the prior art in its superior qualities as an electrical system for bodily tissue. A wire or needle tip or a flat or smooth metal contact has only a small surface area to inject current capacitively. In one embodiment of the present disclosure, a conductive hydrogel can cover large areas for example after surgery of brain tumors. The charge injection in an implanted electrode may consist of both capacitive and resistive current transfer. In a bodily tissue, in some circumstances, the best way to inject current is via capacitive charge injection, which does not lead to irreversible chemical reactions, but during other circumstances the injection and removal of electrons in redox reactions can be preferred; in some circumstances, a combination of capacitive charge injection and redox reaction are preferred.

The present invention, comprises a variety of material specific physical parameters including, without limitation, curing inside the body, from flexible to stiff and/or rigid post cure, with different conductivities and the ability to mechanically interface with nearby locations within the body next to the target organ to have additional stress and/or strain relief on both, an organ and on a cured electrode post placement.

In contrast to prior art electrodes, whose microscopic surface structure and macroscopic shape is formed ex-vivo, the electrode disclosed herein receives both it microscopic surface structure and macroscopic shape in-vivo: by adapting to target similar to how a cast forms as a mold around an arm or leg. This is achieved by one or more processes of manufacturing the electrode in-vivo either inside a living organism or on the outside of a living organism. Although the electrode may be formed inside the body fully or in part, it may also be formed on the outside without touching

The developed (cured) electrode, once set, can be strategically positioned to engage with blood vessels, enabling it to either stimulate or inhibit signal pathways within the vessel walls.

This innovative approach allows for the direct injection of a liquid compound surrounding a blood vessel to manage blood flow, potentially tightening or loosening the vessel to adjust blood delivery to organs, tissues, or the skin. This regulation can enhance circulation or limit heat loss as needed. In particular applications, this technology can target blood vessels supplying tumors, aiming to restrict or halt blood flow to these areas. This method effectively deprives the growth of essential nutrients and oxygen, potentially slowing or even reversing undesirable cellular proliferation. The process involves using a catheter to deliver the mixture, either within the blood vessel wall or around it, establishing electrical contact from the vessel's exterior through a connected wire. Alternatively, the electrode compound can be administered from a distance, gradually nearing the vessel to form a ring around it. This ring, created by piercing the vessel wall from inside out, can either form a complete or segmented encirclement. A wire component of the electrode, inserted separately, then establishes the necessary electrical connection to precisely target areas within the body or just under the skin, enhancing the treatment's effectiveness and specificity.

The developed (cured) electrode, once set, can be strategically positioned to engage with blood vessels, enabling it to either stimulate or inhibit signal pathways within the neuron of the CNS or PNS, as capillary systems are <100 μm distance from neurons.

The present disclosure, in some embodiments, may be placed into, near, or around an organ, especially specific structures of an organ such as internal blood vessels or neurons, or an inside or outside wall of the organ to be capable of electrical stimulation, or blockage of signal transmission, in the organ, the innervation or the blood supply of the organ, for example, the bladder. Organ activity can be changed by increasing or decreasing neural communication into and out of the organ, and some organ growth and activity can be up- or down-regulated by allowing more or less blood enter the organ, such in the case of the gut, the liver, the lungs or the kidney which are exchange systems for the body, utilizing a fine mesh of blood vessels intertwined with other vessels who either add or extract chemicals in the form of dissolved gasses or liquids. The present invention allows for an efficient way to contact an organ, such as by injecting the liquid mixture to the outside wall of an organ near an innervation point.

Beyond medical treatments, the present disclosure also encompasses energy storage applications, where the unique properties of the composition of the first aspect can be utilized in bioelectronic devices. This includes creating more efficient, biocompatible batteries or capacitors for medical implants and other bioelectronic devices, highlighting the versatile potential of these compounds in both healthcare and technology sectors. Thus, use of a compostions according to the first aspect may be in energy storage applications, such as bioelectronic devices or other biocompatible devices. The bioelectronic devices may for example be biocompatible batteries or capacitors for medical implants.

### Definitions

As used herein, the term “C₁₋₆ alkyl” means both linear and branched chain saturated hydrocarbon groups with 1 to 6 carbon atoms, while the term “C₁₋₂₀ alkyl” refers to linear or branched chain saturated hydrocarbon groups with 1 to 20 carbon atoms. The term “C₁₋₆ alkoxy” means the group O—C₁₋₆ alkyl, where “C₁₋₆ alkyl” is used as described previously. The term “C₃₋₆ cycloalkyl” means a cyclic saturated hydrocarbon group, with 3 to 6 carbon atoms. The term “halogen” means fluorine, chlorine, bromine or iodine. The term “C₂₋₆ alkenyl” refers to a straight or branched chain unsaturated hydrocarbon containing 2-6 carbon atoms. The “alkenyl” group contains at least one double bond in the chain. The double bond of an alkenyl group can be unconjugated or conjugated to another unsaturated group. The term “C₂₋₆ alkynyl” refers to a straight or branched chain unsaturated hydrocarbon containing 2-6 carbon atoms. The “alkynyl” group contains at least one triple bond in the chain.

Unless otherwise specifically defined, the term “aryl” refers to cyclic, aromatic hydrocarbon groups that have 1 to 3 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl).

As used herein, the term “heteroaryl” means a monocyclic aromatic group of carbon atoms wherein from one to three of the carbon atoms is/are replaced by one or more heteroatoms, such as nitrogen, oxygen and/or sulfur. Examples of monocyclic heteroaryl groups include, but are not limited to, furyl, thienyl, pyrrolyl, oxazolyl, thiazolyl, imidazolyl, oxadiazolyl, thiadiazolyl, pyridyl, triazolyl, triazinyl, pyridazyl, isothiazolyl, isoxazolyl, pyrazinyl, pyrazolyl, and pyrimidinyl.

As used herein, the term “heterocyclyl” means a cyclic group of carbon atoms wherein from one to three of the carbon atoms is/are replaced by one or more heteroatoms, such as nitrogen, oxygen and/or sulfur. Examples of heterocyclyl groups include, but are not limited to tetrahydrofuryl, tetrahydropyranyl, pyrrolidinyl, piperidinyl, piperazinyl, morpholinyl, and dioxanyl.

Herein the term “cure” relates to a chemical process that includes, without limitation, polymerizing, crosslinking, going through precipitation and/or going through self-organisation, gelation or other phase transition to a conductive which retains its shape when subjected to shear forces expected for a living body in non-extreme conditions. The curing may be substantially instantaneous, a few seconds or minutes, or may occur over a longer period of time.

“Inject” means introducing into bodily tissue through (a) a dispenser by means of a needle or needle-like structure without the need of an incision besides that of the needle, (b) a catheter in a blood vessel or other bodily structure with a lumen, (c) a pump through a laparoscopic device inserted through a small incision, (d) a hole that has been created with a separate incision, or (e) an auger system transporting the injectable material inside a lumen from which it is expressed near, into or around an interface target. In another term inject means using a needle-free jet injector to inject the electrode solution by contacting a surface of the human body or organ to form a conductive structure in the body or organ.

The needle-based, needle-free jet injector, endovascular catheter, and laparoscopic approach to placing a liquid mixture resulting in a cured electrode allow for a dorsal surgical approach to connecting to organs in novel ways, similar to the ability to connect to intercostal nerves and ganglia of the autonomic nervous system.

A “patient” or “subject” is a mammal, e.g., a human, mouse, rat, guinea pig, dog, cat, horse, cow, pig, or non-human primate, such as a monkey, chimpanzee, baboon or rhesus.

An “effective amount” when used in connection with a compound is an amount effective for treating or preventing a disease in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed composition to a subject, or administering a prodrug derivative or analog of the compound or pharmaceutically acceptable salt of the compound or composition to the subject, which can form an equivalent amount of active compound within the subject's body.

Depending on the substituents present in compounds of the formula (I), (I-1), (I-a), (1-b), (I-c), (I-d), and (I-e), formula (II) and (Ila), and formula (III), (III-a), (III-b), (III-c), and (III-d), the compounds may form salts which are within the scope of the present disclosure. Salts of said compounds, which are suitable for use in medicine are those wherein a counterion is pharmaceutically acceptable.

Suitable salts according to the present disclosure include those formed with organic or inorganic acids or bases. In particular, suitable salts formed with acids according to the present disclosure include those formed with mineral acids, strong organic carboxylic acids, such as alkanecarboxylic acids of 1 to 4 carbon atoms which are unsubstituted or substituted, for example, by halogen, such as saturated or unsaturated dicarboxylic acids, such as hydroxycarboxylic acids, such as amino acids, or with organic sulfonic acids, such as (C1-C4)alkyl or aryl sulfonic acids which are unsubstituted or substituted, for example by halogen.

Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine.

Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucamine, morpholine, thiomorpholine, piperidine, pyrrolidine, a mono, di- or tri lower alkylamine, for example ethyl, tertbutyl, diethyl, diisopropyl, triethyl, tributyl or dimethylpropylamine, or a mono-, di- or trihydroxy lower alkylamine, for example mono-, di- or triethanolamine. Corresponding internal salts may furthermore be formed.

The compounds of the present disclosure may be used in the prophylaxis and/or treatment as such, or in a form of a pharmaceutical composition. While it is possible for the active ingredient to be administered alone, it is also possible for it to be present in a pharmaceutical composition. Accordingly, the present disclosure provides a pharmaceutical composition comprising a polymer of formula (I) and one or more compounds of formula (II) or formula (III), and a pharmaceutically acceptable diluent, excipient and/or carrier. Pharmaceutical compositions of the present disclosure may take the form of a pharmaceutical composition as described below.

The polymer may be one of the different types of polymers selected from the group consisting of: homopolymer, alternating co-polymer, random co-polymer and block co-polymer. In the polymer of formula (I), the number of repeating units is annotated with n and m, wherein n is between 4 and 32, preferably between 5 and 12, and m is between 0 and 10, preferable between 0 and 3. When m is 1 as in the compound of formula (I-a), then p is between 1 and 12, preferably between 5 and 12, a is between 1 and 5, E is preferably H, and R₁ is H or C₁₋₆ alkyl, such as methyl. Further examples are compounds of formula (I-b-1) and formula (I-c-1). The compound of formula (I-b-1) with A being sodium is annotated as compound (1-1) and herein referred to as PEDOT-S derivative A5 or PEDOT-S(A5). The compound of formula (I-c-1) is herein referred to as Ok-PEDOT-S. Any of the previously mentioned examples of compounds with m=1 can be transformed to equivalent compound with E being selected from —(CH₂CH₂O)_{q}CH₂CH₂OH (q=0-15), C₁₋₆ alkyl substituted with —N₃, C₁₋₆ alkyl substituted with-SO₃A, and C₁₋₆ alkyl substituted with-SO₃A and —N⁺(C₁₋₆ alkyl)₃. These compounds are herein referred as PEDOT-S-E.

Furthermore, the polymer of formula (I) may be a homopolymer. In this case m is 0 and R₁ is selected from H and C₁₋₆ alkyl substituted with —N⁺(C₁₋₆ alkyl)₃. Examples of the polymer of formula (I) with m=0 comprise compound (I-d) with R₁ being hydrogen, and compound (I-e) with R₁ being C₁₋₆ alkyl substituted with —N⁺Me₂(R₁₁), wherein R₁₁ being selected from H and C₁₋₆ alkyl, r being 4-30, a being 1-4, and b being 1-3. Compound (I-e) is herein referred to as PEDOT-SA.

The examples of the polymer of formula (I) with m=1 thus comprise the following:

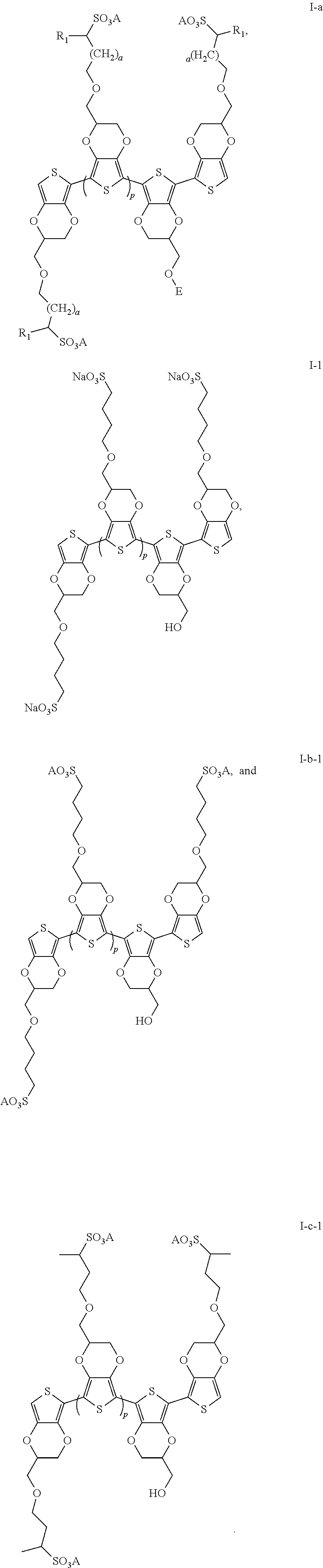

The examples of the polymer of formula (I) with m=1 thus comprise the following:

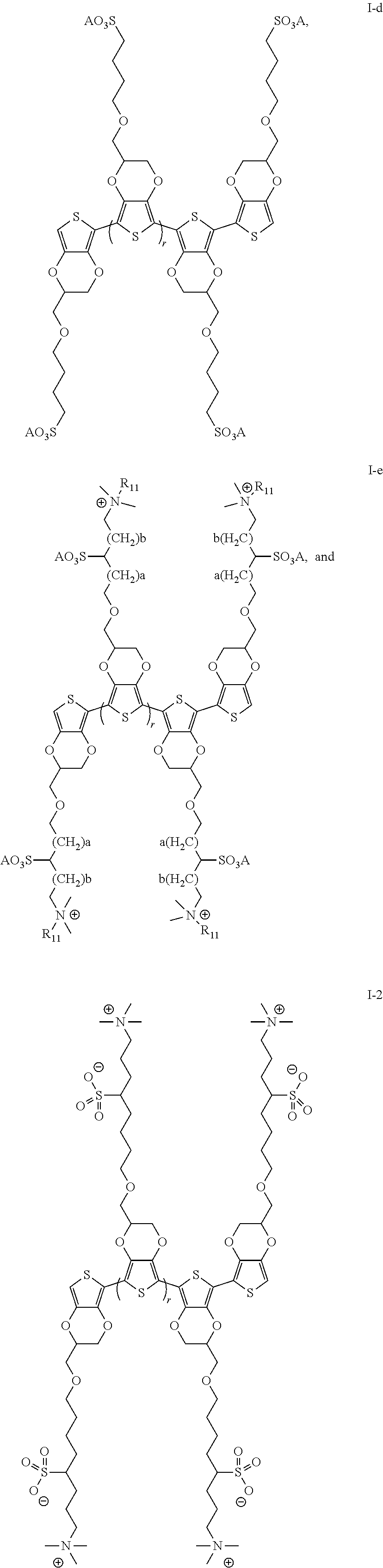

The polymer of formula (I) may be self-doped, water-soluble and a mixed ion-electron conductor of a poly(3,4-ethylenedioxythiophene) butoxy-1-sulfonate (PEDOT-S) derivative. Among the PEDOT-S derivatives, such as A5, are unique because the polymer self-assembles in an agarose gel cast with a physiological buffer and generates a highly conductive hydrogel (1-5 S cm⁻¹). PEDOT-S is also stable for several months. On average, PEDOT-S derivatives comprise small polymers, i.e., oligomers of 7-8 monomers. Thus, PEDOT-S derivatives, e.g. A5, is smaller than the antisense oligonucleotide drugs which contains about 20 nucleotides. The polymer of formula (I) is expected to have better bioresorption properties than PEDOT:PSS, where the PSS part is a large polymer of 200-300 monomers with a Mₙ about 70 000 g/mol. The polymer of formula (I) may be in the form as highly water-dispersed nanoparticles. Further, the polymer of formula (I) may be a self-doped p-type conducting copolymer.

The one or more compound of formula (II) or formula (III) may be more specifically represented by compound of formula (II-a) and (III-a)-(III)-d as previously described. Said compounds may also be referred as trimers of either ETE-R derivatives, EEE-R derivatives, or TET-R derivatives, wherein the “E” stands for a monomer structure of a 3,4-ethylenedioxythiophene (EDOT) and the “T” stands for a monomer structure of a thiophene. The term “R” in these trimer structures means that the trimers may be optionally substituted with one or more substituents which are not explicitly expressed by a replaced “R” but just indicative of the type of substitution. The trimers, e.g., ETE-R, EEE-R or TET-R, are designed to have a lower oxidation potential than the EDOT monomer: 0.3-0.5 V and 1.2 V, respectively, which is significant for minimal damage to the tissue during electropolymerization. In addition to the structural modification, i.e., shape and functionality of the compounds, and increased in vivo stability, conductivities of two to three orders of magnitude higher than tissue were measured. For example, ETE-Rs may provide the functionalization of choice and facilitate reaching neurons where the initially formed electrodes would not. Some examples of the ETE-Rs comprise previously mentioned sodium 4-(2-(2,5-bis(2,3-dihydrothieno[3,4-b][1,4]dioxin-5-yl)thiophene-3-yl)ethoxy)butane-1-sulfonate (compound II-1, herein referred to as ETE-S) and compound II-2 (herein referred to as ETE-PC). Other examples of the ETE-Rs, but also of the EEE-R derivates and the TET-R derivates will be shown in the next section.

ETE-R derivatives or may be mixed into a solution of polymer (I), e.g., A5, such as an aqueous solution. Other solvents such as organic solvents, e.g. acetone, acetonitrile, butanone, dimethyl formamide, dimethyl sulfoxide, methanol, ethanol, isopropanol, glycerol, polyethylene glycol (PEG-400), propylene glycol, may also be possible to use. The concentration of polymer in solution may be in the range of 1-100 g/ml. The solution may be injected into the brains, and electrofunctionalized, forming a mixed ion-electron conducting hydrogel, with different properties depending partly on the R substituents on one or several ETEs. With the term “hydrogel” means a biphasic material comprising a mixture of porous, permeable solids, e.g. insoluble three dimensional network of natural or synthetic polymers, and at least 10% by weight or volume of interstitial fluid composed completely or mainly by water. Thus, hydrogels may be described as polymeric network structures that are able to imbibe large amount of water.

With the term “Mixed ion-electron conducting hydrogel” means that the electrode conduct both ions and electrons.

The conductive hydrogel is transient, and the initial inflammation in the brain caused by the injection resolves, leaving no tissue damage from the electrode behind. Hence, ETE-Rs provide the functionalization of choice and facilitate reaching neurons where the electrodes of the prior art would not.

With the term “transient bioelectronics” or “transient organic bioelectronics” means devices or compositions that are bioresorbable, i.e., that disappears from the environment after a programmed time and leaves minimal and harmless traces after their disposal. Depending on the structure of the composition the bioresporption process, i.e. the degradation of the composition in vivo may be in the range of 1-5 days, 1-14 days, 1-60 days, 1-120 days, 1-240 days or even up to 1 year. Methods for in vivo polymerization of the composition according to the first aspect of the present disclosure may be electropolymerization, photopolymerization, i.e., light-induced polymerization, such as visible light, preferably blue (450-495 nm) and/or green (495-570 nm) visible light, ultraviolet light and/or infrared light, or enzymatic polymerization, i.e., polymerization using enzymes, such as endogenous catalases and peroxidase, e.g. horseradish peroxidase (HRP), Myeloperoxidase (MPO), and lactoperoxidase (LPO).

### Methods for Synthesizing the Compounds and Compositions

The compounds and compositions of the present disclosure may be made by a variety of methods, including standard chemistry. Suitable synthetic routes are depicted in the schemes given below.

The polymers of formula (I) and the compounds of formula (II)-(II) may be prepared by methods known in the art of organic synthesis as set forth in part by the following synthetic schemes. In the schemes described below it is well understood that protecting groups for sensitive or reactive groups are employed where necessary in accordance with general principles or chemistry.

The compounds described herein may be made from commercially available starting materials or synthesized using known organic, inorganic, and/or enzymatic processes. Another aspect of the present disclosure provides a process for preparing the polymer of formula (I) and the compounds of formula (II)-(II), or a pharmaceutically acceptable salt thereof, wherein all substituent in, unless specified otherwise, as defined herein. Said process comprises of:

### (i) Preparation of the Polymer of Formula (I-1)

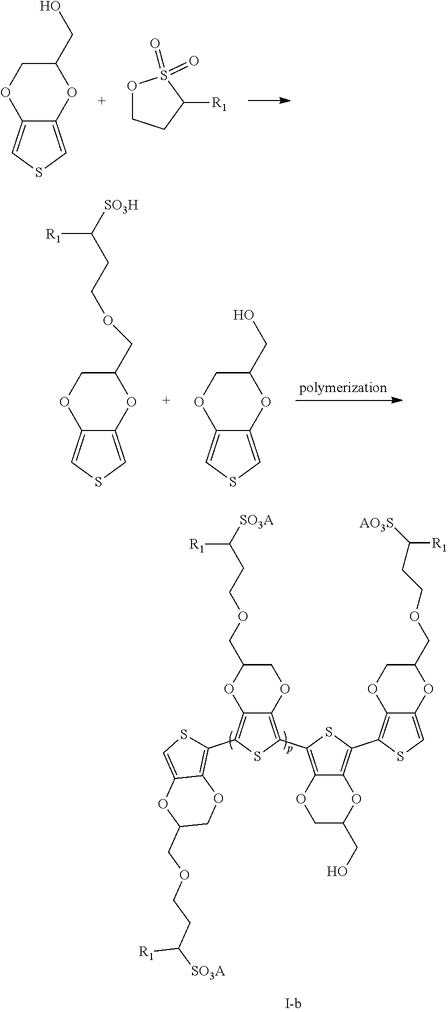

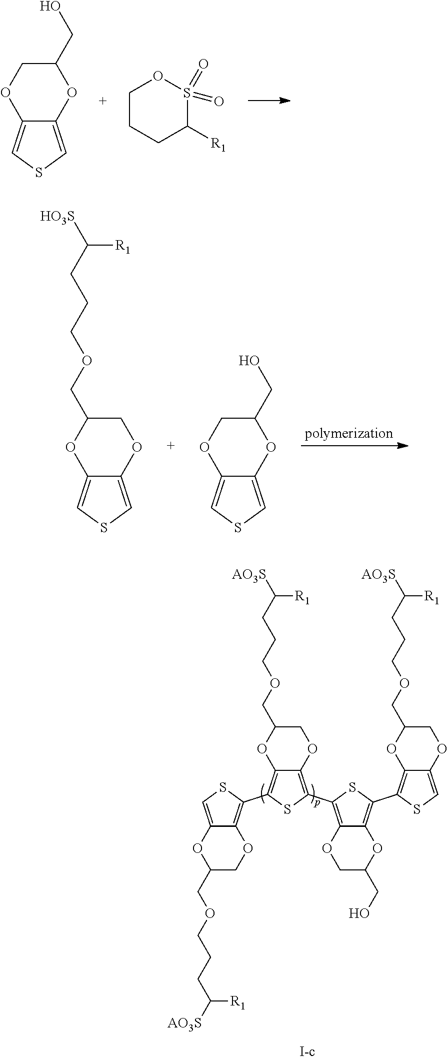

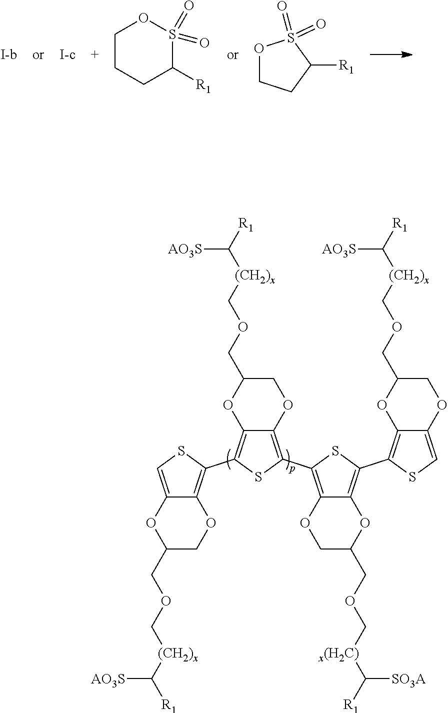

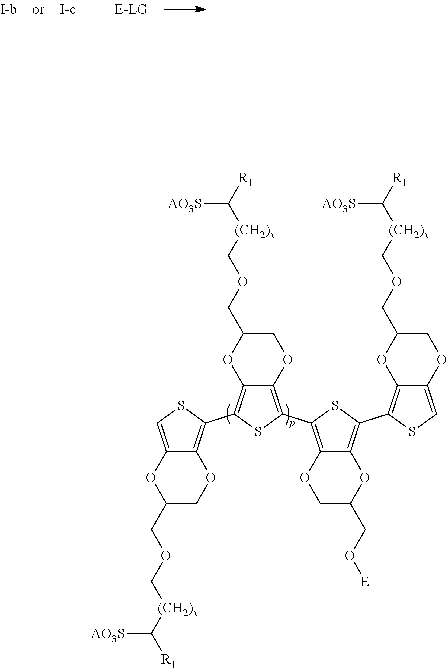

wherein R₁ is as previously defined; E is C₁₋₆ alkyl, C₂₋₆ alkenyl, C₂₋₆ alkynyl, C₃₋₆ cycloalkyl, aryl, heteroaryl, —(CH₂CH₂O)_{q}CH₂CH₂OH, wherein said C₁₋₆ alkyl is optionally substituted with one or more of —N₃, —OH, —SO₃A, —N(C₁₋₆ alkyl)₂, —NH⁺(C₁₋₆ alkyl)₂, and —N⁺(C₁₋₆ alkyl)₃; LG is a leaving group, such as halide, tosylate, mesylate; and x is 2 or 3.

Non-limiting examples of prepared polymers of formula (I) using one or more steps show above comprise: PEDOT-S(A5) (compound I-1); Ok-PEDOT-S(compound I-c-1); compound I-d; PEDOT-SA (compound I-e); PEDOT-BuSA (compound I-2). The polymer of formula (Ib) or (Ic) was synthesis according to Mousa, A. H. et al. (Method Matters: Exploring Alkoxysulfonate-Functionalized Poly(3,4-ethylenedioxythiophene) and Its Unintentional Self-Aggregating Copolymer toward Injectable Bioelectronics. Chemistry of Materials 34, 2752-2763 (2022)) and optionally further substituted polymers of formula (I) may be synthesized by reacting the polymer of formula (Ib) with alkane sultones, e.g. 1,3-propane sultone or 1,4-butane sultone, optionally substituted with R₁, or E-LG as shown above.

As an example, polymers I-2 and I-d were synthetized according to the following reaction schemes:

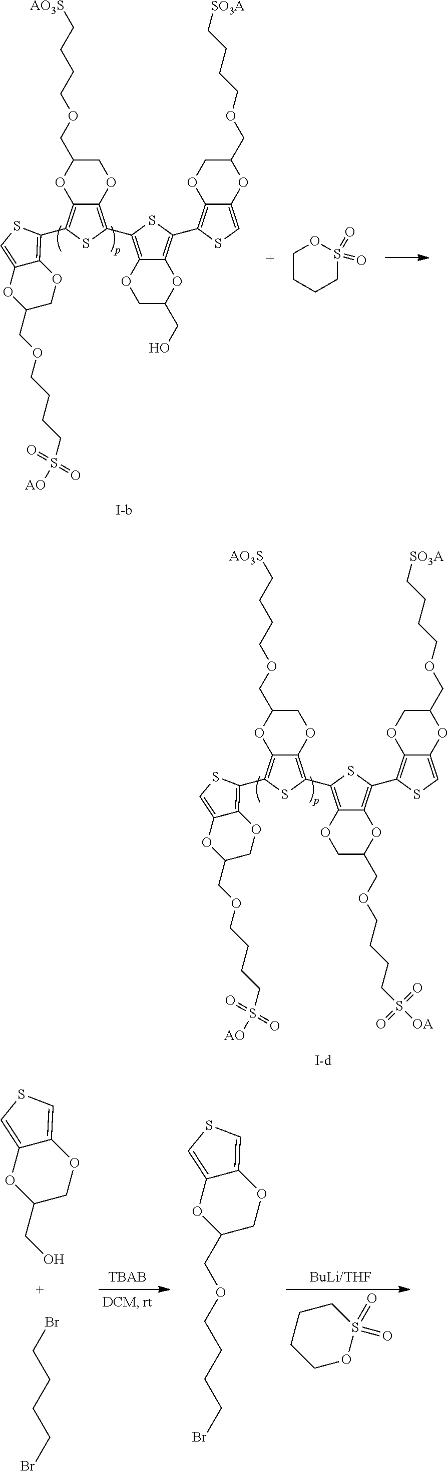

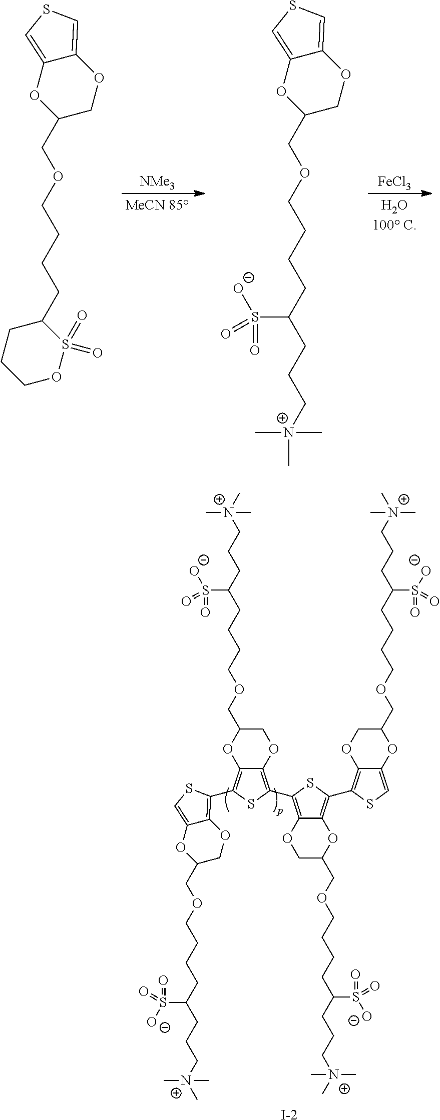

### (ii) Preparation of Compounds of Formula (II)-(III)

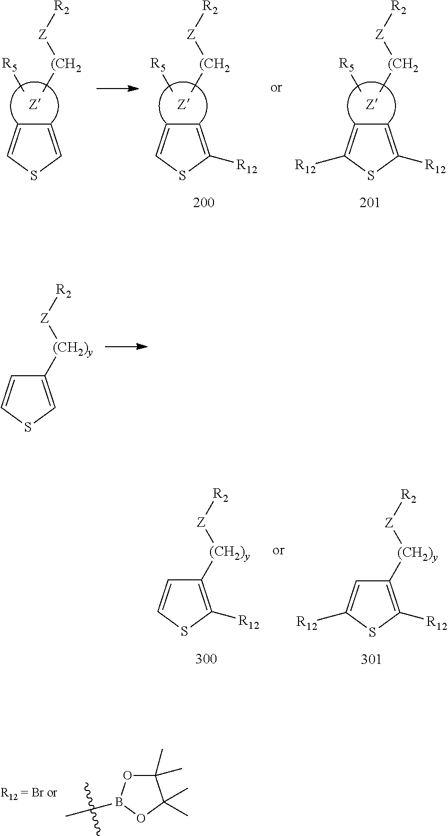

Structures 200, 201, 300 and 301 may be commercially available or synthezied according to known literature procedures, for example mono- or di-bromination of respective thiophen precursors, and optionally further functionalized with boronic acids or boronic esters, such as Bis(pinacolato)diboron. The compounds of formula (II) or (III) may be synthesized from structures 200, 201, 300 and 301 according to the following schemes using Suzuki coupling or any other alternative method known to the skilled person.

Non-limiting examples of prepared compounds of ETE-R derivates using one or more steps shown comprise:

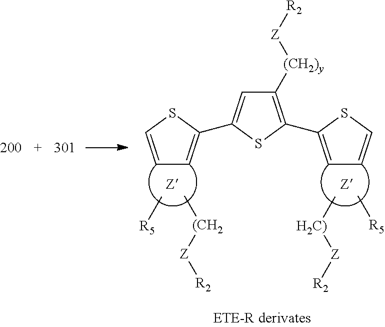

ETE-R derivates: ETE-S(compound II-1), ETE-PC (compound II-2), ETE-BuSultone (compound II-3), ETE-BuSA (compound II-4),

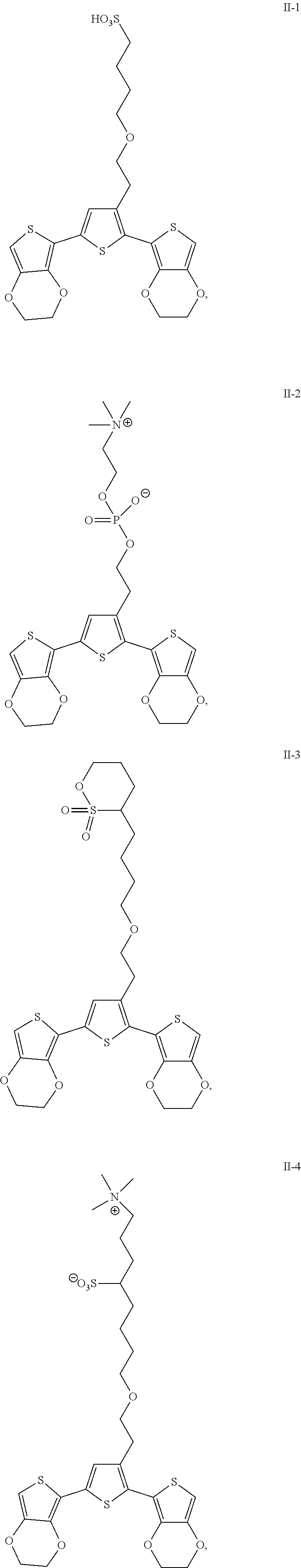

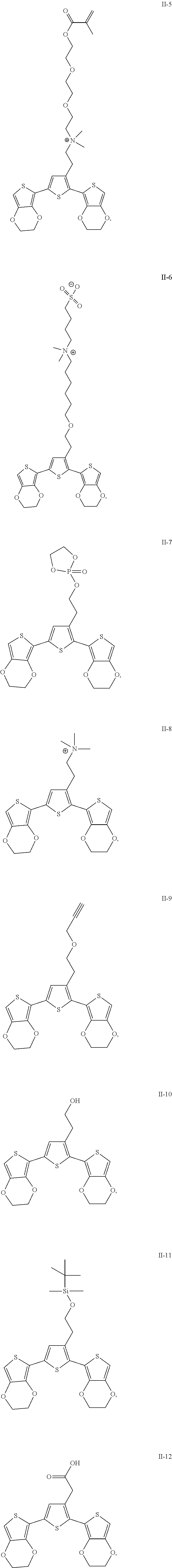

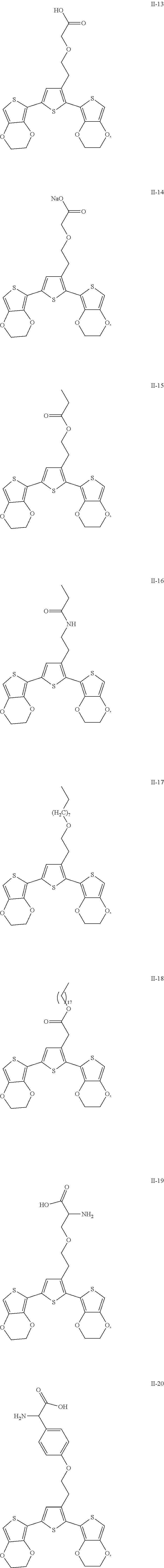

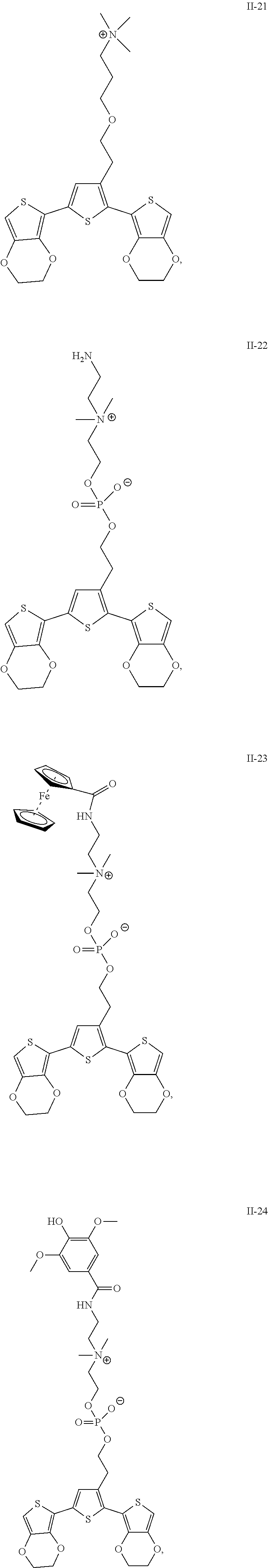

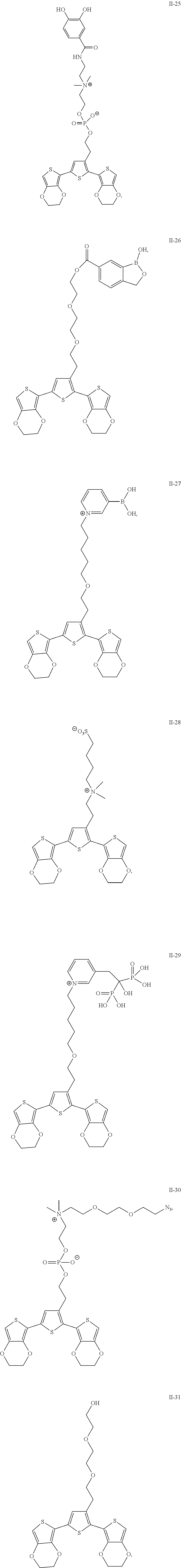

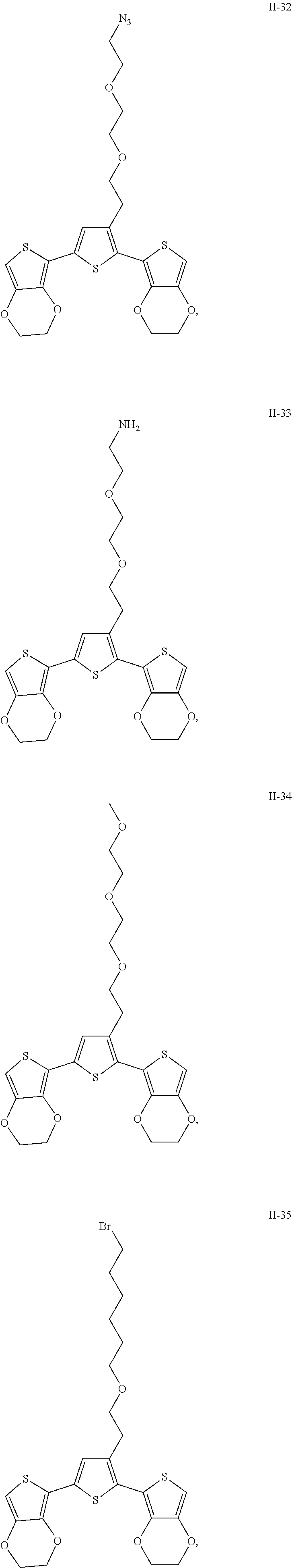 [SMILES: [N-]=[N+]=NCCOCCOCCc1cc(-c2scc3c2OCCO3)sc1-c1scc2c1OCCO2 ; NCCOCCOCCc1cc(-c2scc3c2OCCO3)sc1-c1scc2c1OCCO2 ; COCCOCCOCCc1cc(-c2scc3c2OCCO3)sc1-c1scc2c1OCCO2 ; BrCCCCCCOCCc1cc(-c2scc3c2OCCO3)sc1-c1scc2c1OCCO2]

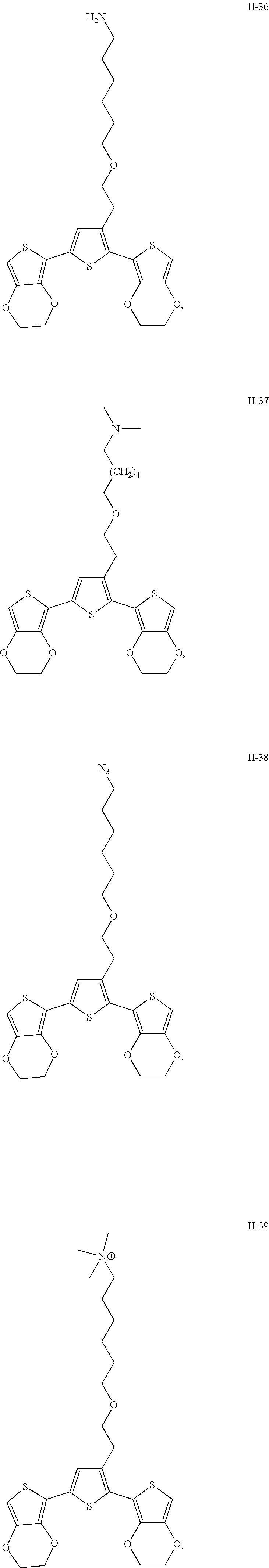

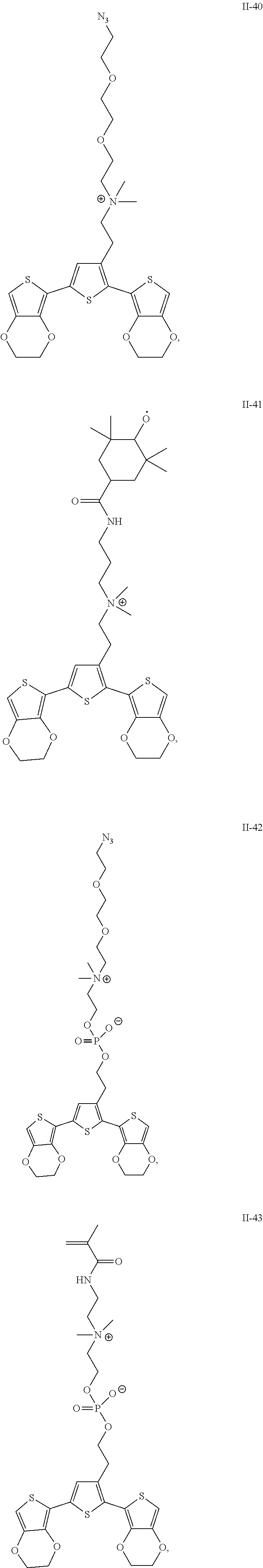

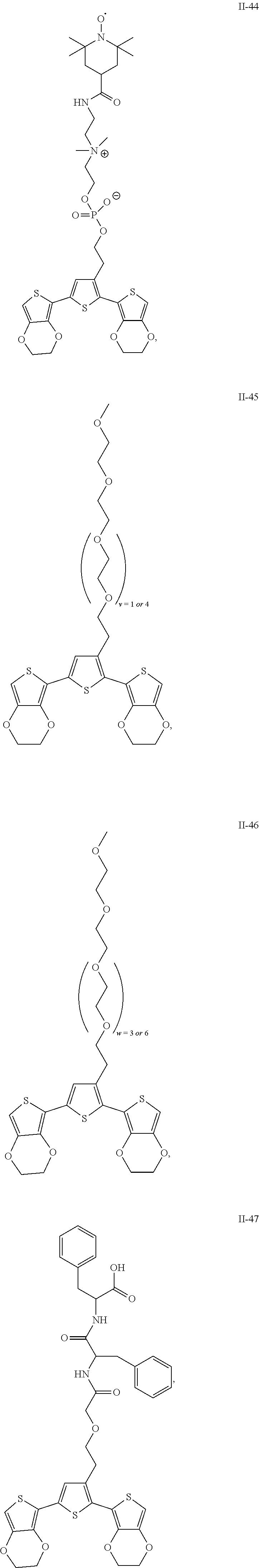

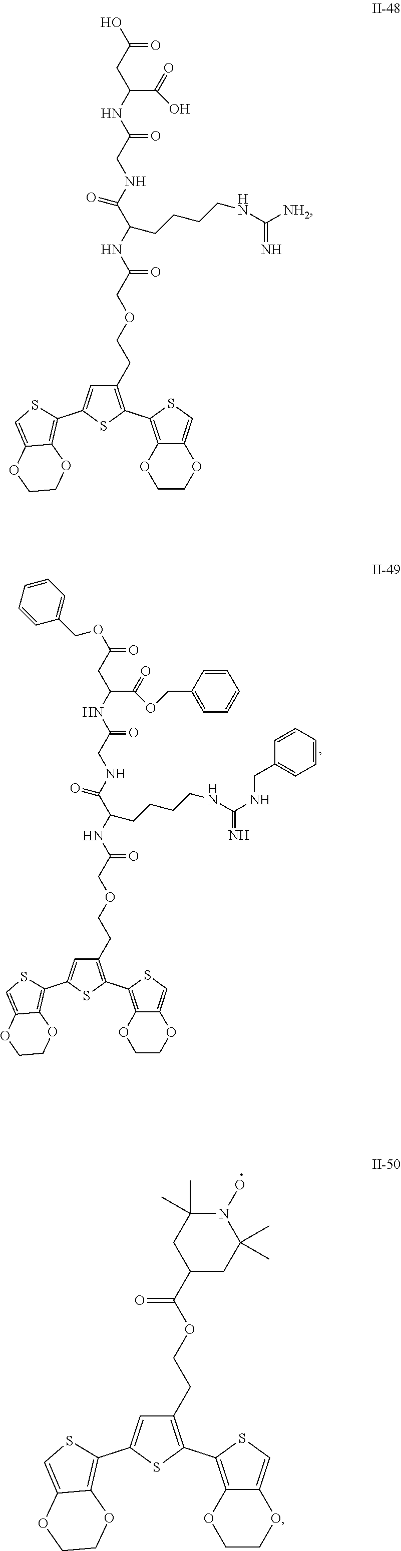

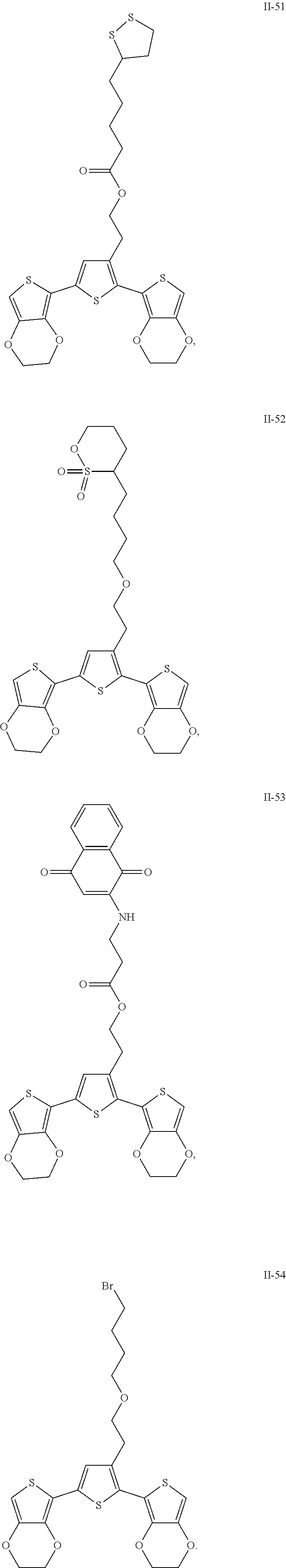 [SMILES: O=C(CCCCC1CCSS1)OCCc1cc(-c2scc3c2OCCO3)sc1-c1scc2c1OCCO2 ; O=S1(=O)OCCCC1CCCCOCCc1cc(-c2scc3c2OCCO3)sc1-c1scc2c1OCCO2 ; O=C(CCNC1=CC(=O)c2ccccc2C1=O)OCCc1cc(-c2scc3c2OCCO3)sc1-c1scc2c1OCCO2 ; BrCCCCOCCc1cc(-c2scc3c2OCCO3)sc1-c1scc2c1OCCO2]

In an embodiment, the ETE derivate may be selected from one or more of II-1, II-2, II-14, II-22, II-23, II-24, II-25, II-28, II-30, II-37, II-39, II-40, II-43, II-44, and II-45.
As an example, the compound II-4 was synthetized according to the following reaction scheme:

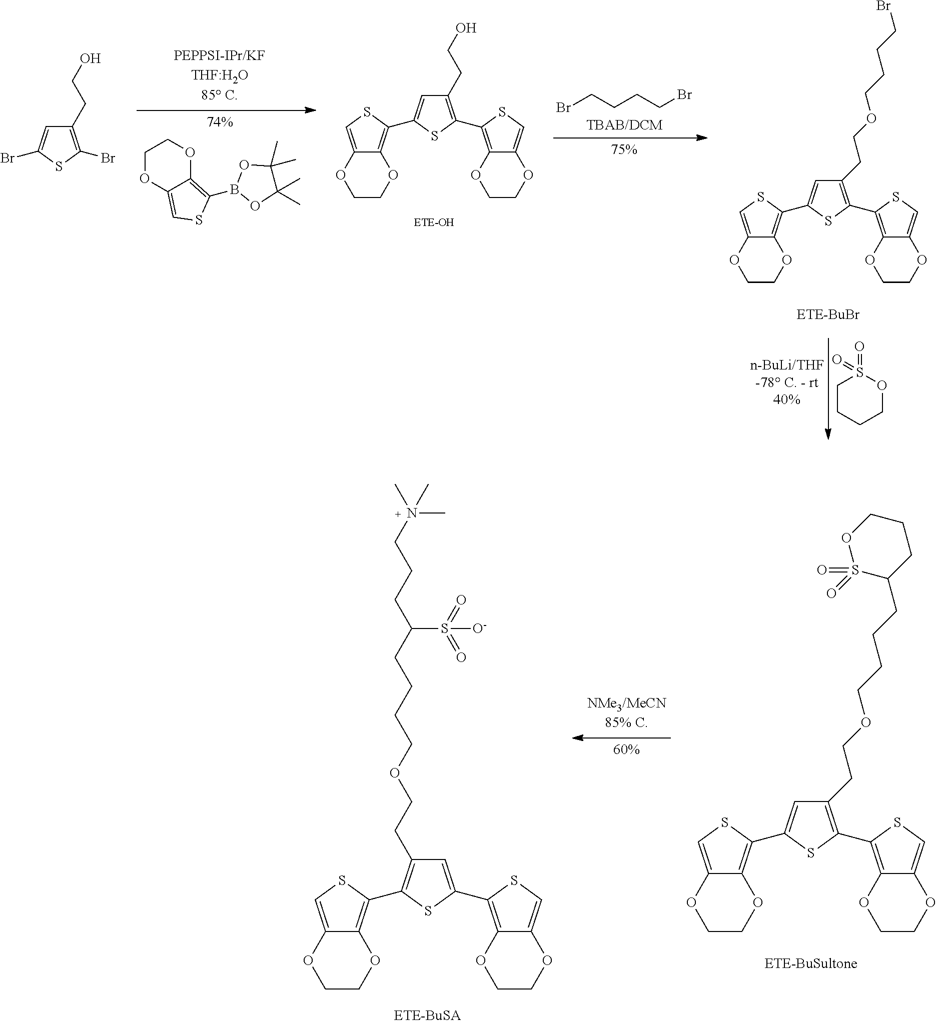

Non-limiting examples of prepared compounds of EEE-R derivates or EPE-R derivates using one or more steps shown comprise:

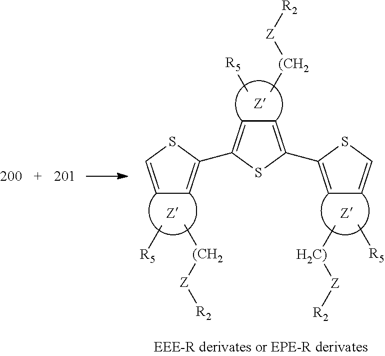

### EEE-R Derivates:

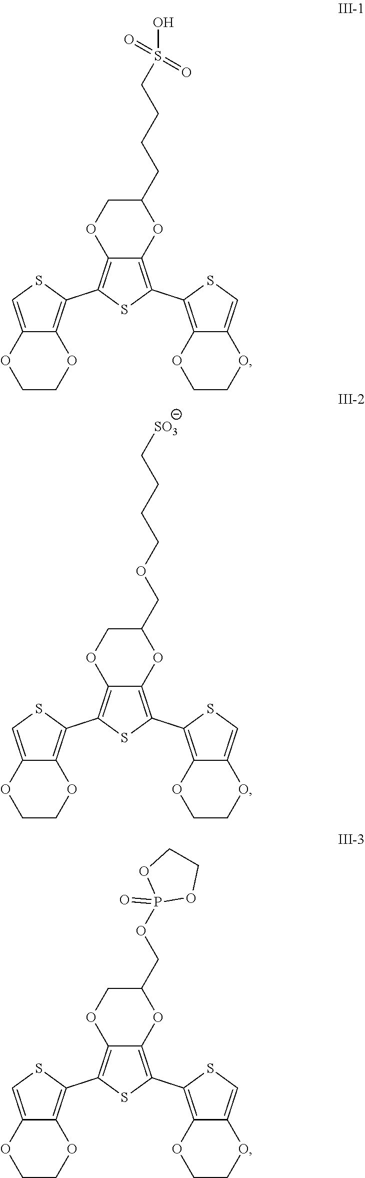

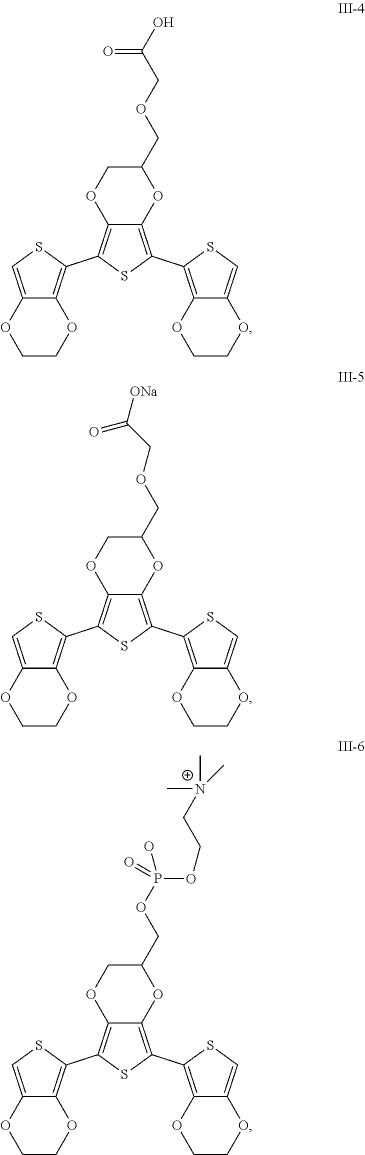

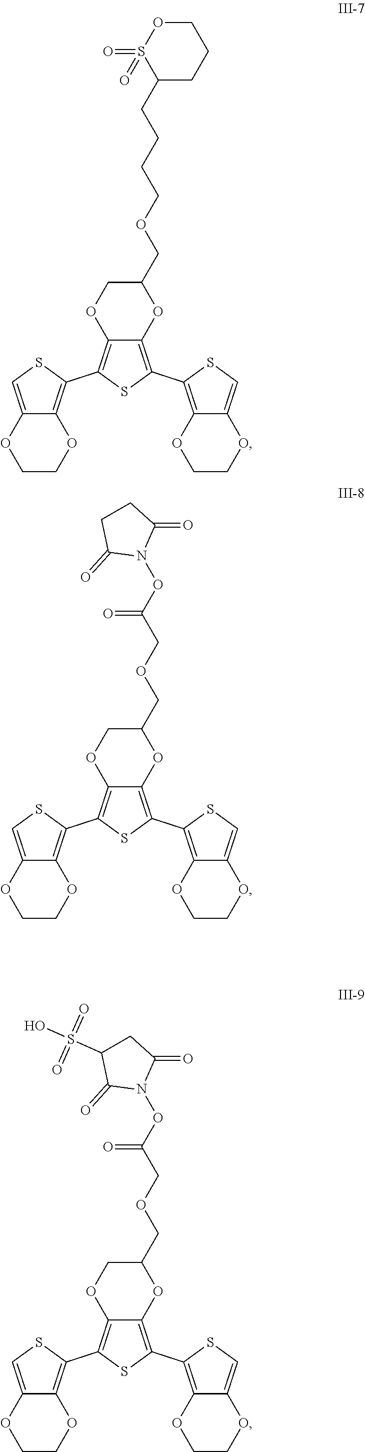 [SMILES: O=S1(=O)OCCCC1CCCCOCC1COc2c(-c3scc4c3OCCO4)sc(-c3scc4c3OCCO4)c2O1 ; O=C(COCC1COc2c(-c3scc4c3OCCO4)sc(-c3scc4c3OCCO4)c2O1)ON1C(=O)CCC1=O ; O=C(COCC1COc2c(-c3scc4c3OCCO4)sc(-c3scc4c3OCCO4)c2O1)ON1C(=O)CC(S(=O)(=O)O)C1=O]

In an embodiment, the EEE derivate may be selected from one or more of III-2, III-5, and III-6.

### EPE-R Derivates:

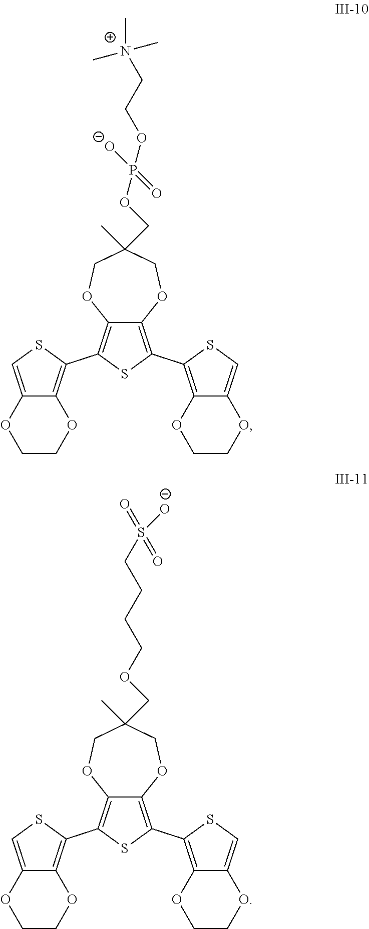 [SMILES: CC1(COP(=O)([O-])OCC[N+](C)(C)C)COc2c(-c3scc4c3OCCO4)sc(-c3scc4c3OCCO4)c2OC1 ; CC1(COCCCCS(=O)(=O)[O-])COc2c(-c3scc4c3OCCO4)sc(-c3scc4c3OCCO4)c2OC1]

Non-limiting examples of prepared compounds of TET-R derivates using one or more steps shown comprise:

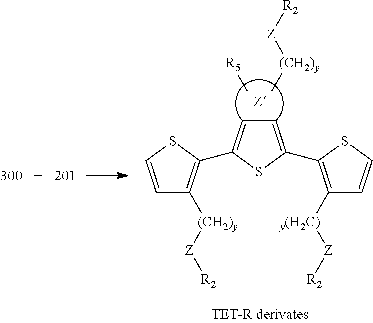

### TET-R Derivates:

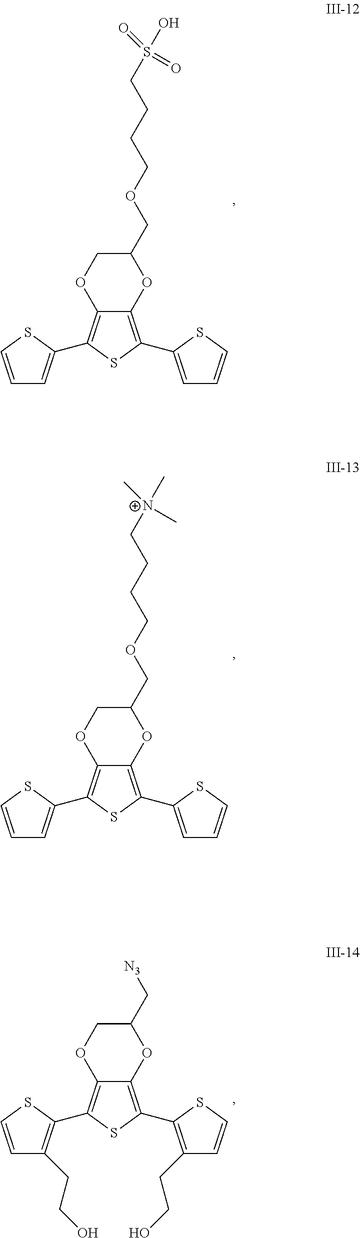 [SMILES: O=S(=O)(O)CCCCOCC1COc2c(-c3cccs3)sc(-c3cccs3)c2O1 ; C[N+](C)(C)CCCCOCC1COc2c(-c3cccs3)sc(-c3cccs3)c2O1 ; [N-]=[N+]=NCC1COc2c(-c3sccc3CCO)sc(-c3sccc3CCO)c2O1]

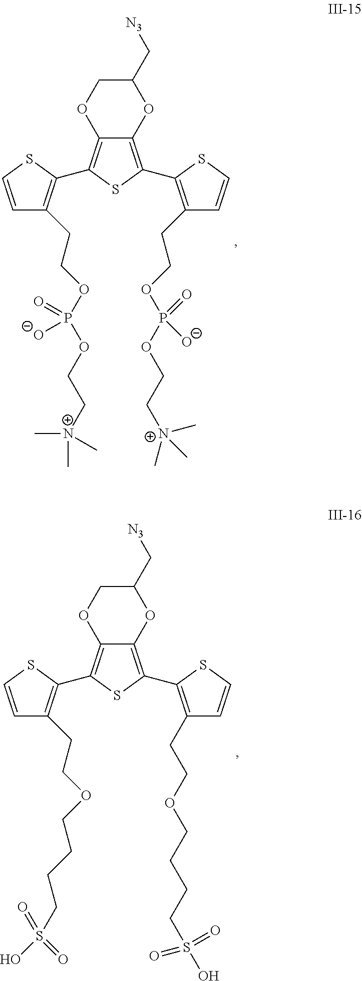 [SMILES: C[N+](C)(C)CCOP(=O)([O-])OCCc1ccsc1-c1sc(-c2sccc2CCOP(=O)([O-])OCC[N+](C)(C)C)c2c1OCC(CN=[N+]=[N-])O2 ; [N-]=[N+]=NCC1COc2c(-c3sccc3CCOCCCCS(=O)(=O)O)sc(-c3sccc3CCOCCCCS(=O)(=O)O)c2O1]

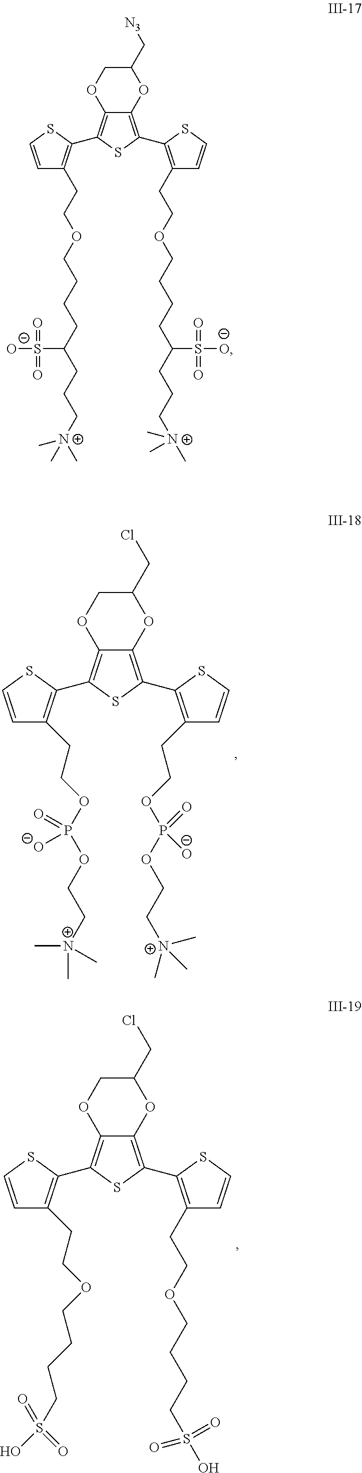

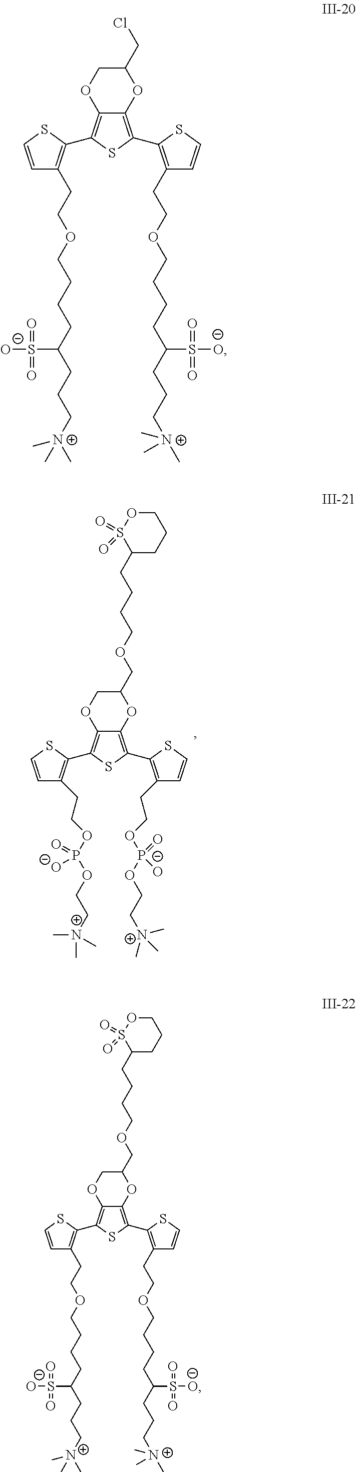

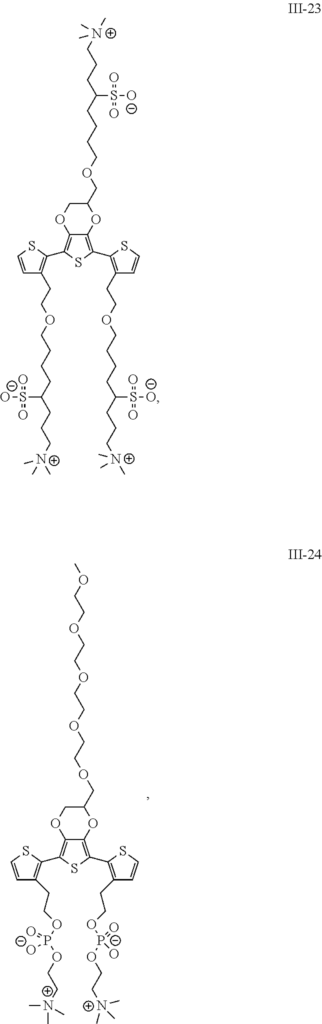

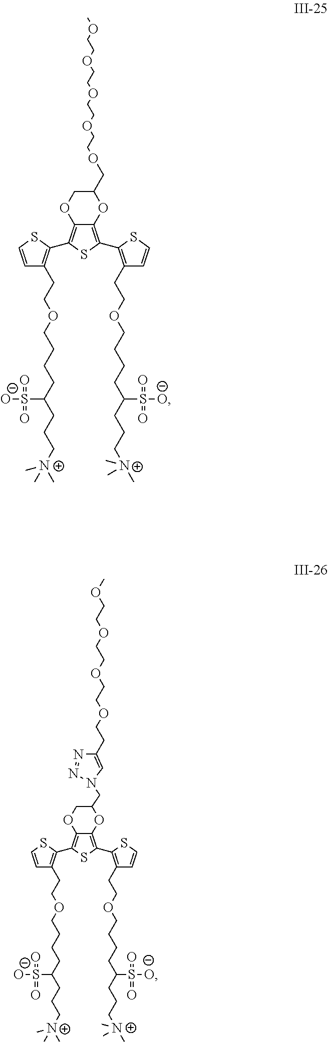

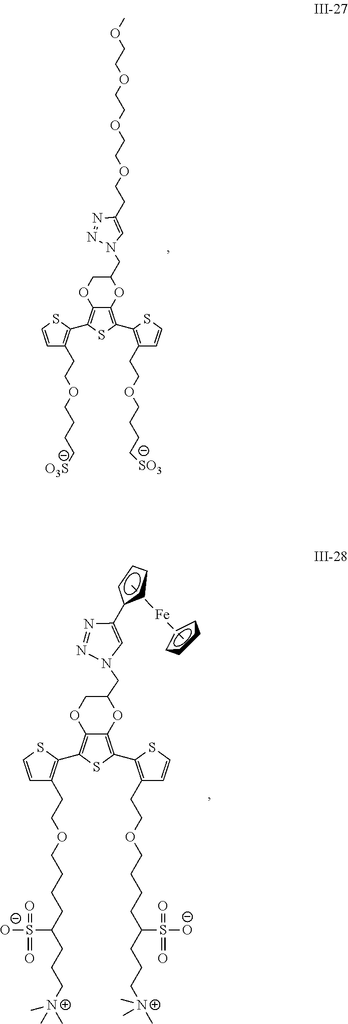

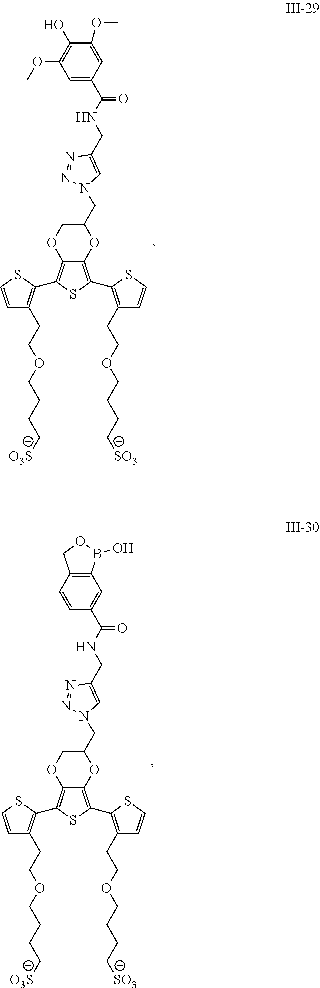

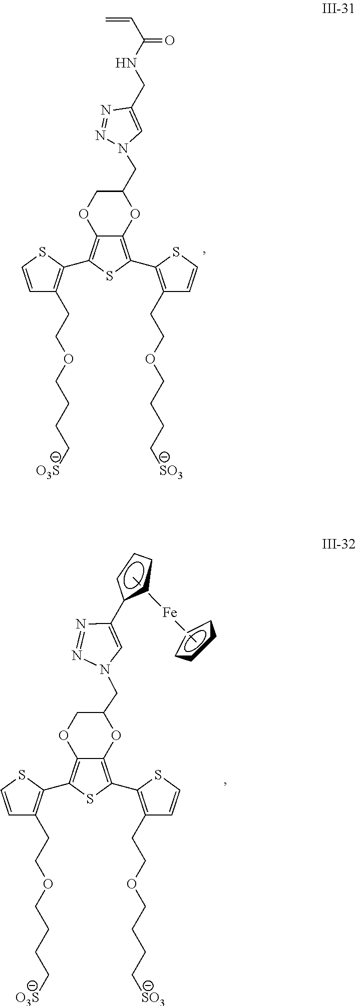

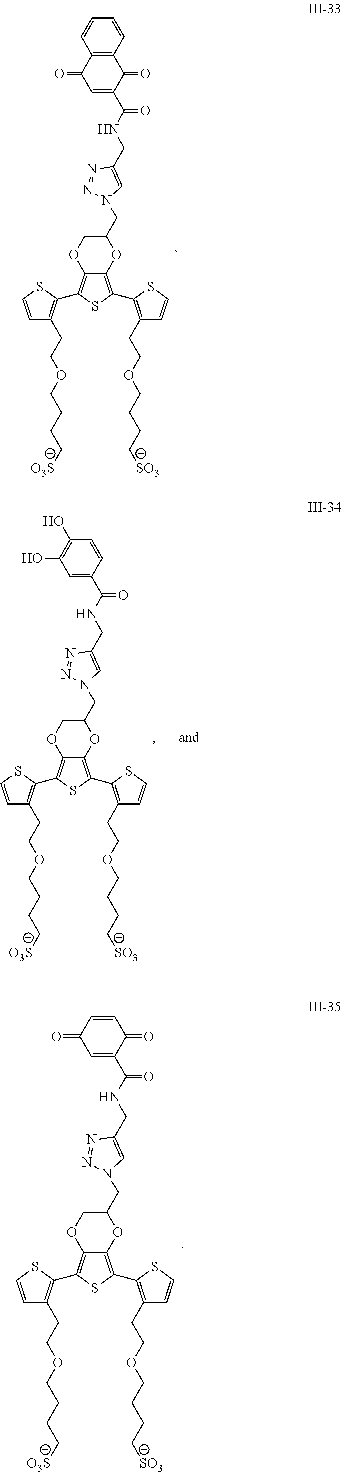

As an example, the compound III-32 was synthetized according to the following reaction scheme:

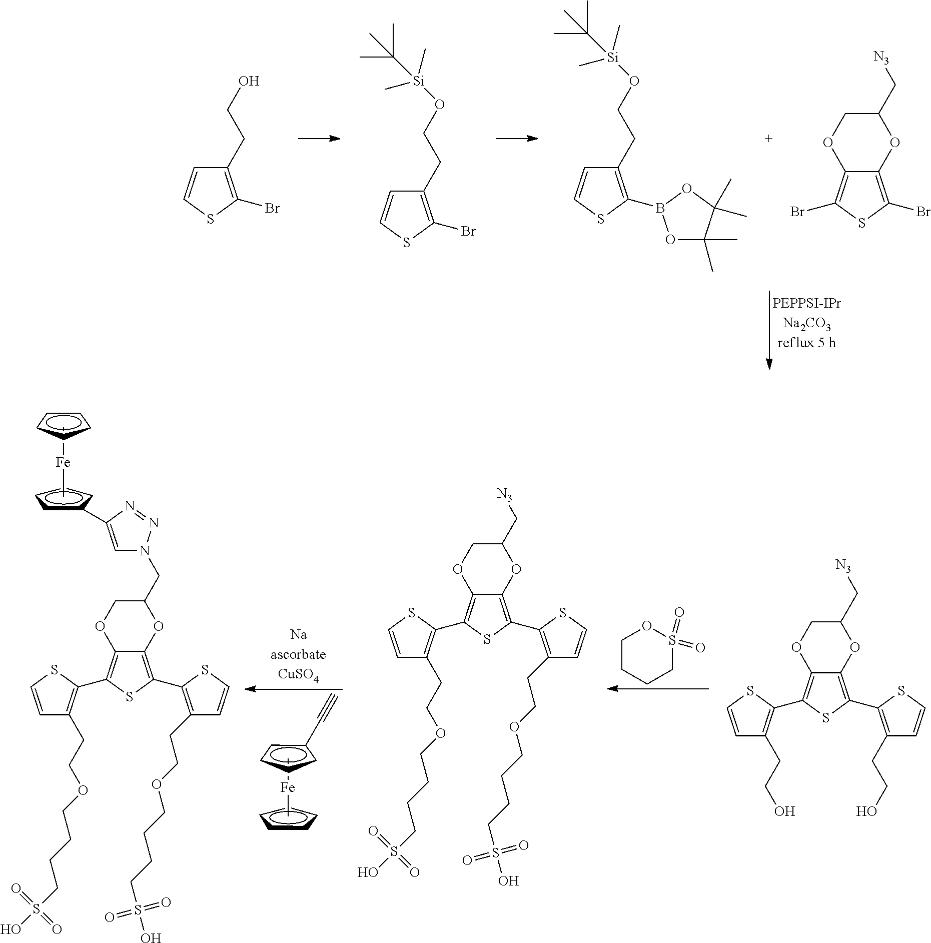

### The Polymer of Formula (I) Self-Assembles to Form a Conductive Electrode in Tissue

Low-concentration agarose gel cast with physiological buffer (Ringer's solution) mimics brain tissue divalent ions enable the polymer to self-assemble into a long-term stable hydrogel in Ringer's solution-agarose gel showing significantly higher conductivity than the surrounding environment. Thus, the polymer, e.g. A5, may be used as an injectable in vivo electrode.

The zebrafish caudal fin is a model system for limb regeneration and neuropathy; thus it is highly dynamic. It is transparent, enabling direct optical access to an injected polymer. A5 (20 mg mL⁻¹) was injected into the caudal fin of zebrafish between the fin rays. To facilitate the assembly of the soft electrodes in the fin, a 25% Ringer's solution was used to dissolve the A5. The use of a formulated composition with additional ion strength, compared to Milli-Q water, is not necessary when injecting into the zebrafish brain. However, this exemplifies the adaptive nature of A5, matching the nanoparticle formulation of the present inventive composition with specific tissue and making it possible to inject into lower and higher ionic strength regions. Directly after injections, dark-blue coherent structures were formed and could visually be seen between the fin rays.

Electrical Properties of A5 in Peripheral Tissue: Initial measurements showed A5 gel's resistance was significantly lower than that of reference samples, indicating higher conductivity. Specifically, resistance measured at 0.16 MΩ for A5 compared to 0.32 MΩ for controls. Upon drying, A5's resistance further decreased to 0.02 MΩ, enhancing conductivity, while the reference's resistance increased to 1 MΩ. These findings underscore the conductive superiority of A5, attributed to the closer alignment of its oligomers upon drying.

Biodegradation and Biocompatibility: A5 displayed transient and bioresorbable properties in zebrafish models. The conductive structure of A5 partially degraded after one week and completely after four weeks when applied in caudal fins, without affecting the zebrafish behavior or causing fin damage, indicating excellent biocompatibility and leaving healthy tissue post-degradation.

Brain Tissue Application and Injection Method: A5 nanoparticles, designed for brain tissue applications, showcased a unique adaptability. A columnar injection technique was utilized, employing a 30 μm diameter cannula to minimize tissue damage, effectively avoiding blood vessel rupture. Following injection, no detrimental effects were observed up to nine days, proving the method's safety and A5's compatibility with delicate brain tissues.

Inflammatory Response and Healing: Initial post-injection observations showed an inflammatory response, which resolved within nine days, indicating the inflammation was due to the injection process rather than A5 itself. This aspect is vital in demonstrating the polymer's biocompatibility and safety for human applications.

Conductive Properties within Brain Tissue: Despite technical challenges, such as variable electrode contact resistance and complex biological interactions, A5 maintained conductive properties in the brain tissue. Visible under bright field microscopy, A5 formed defined conductive patterns, with a linear current-voltage relationship observed at microscale distances. This performance underlines A5's potential in bioelectronic applications, even within the challenging environment of brain tissue.

Comparative Conductivity and Technical Challenges: Although in vitro conductivity of A5 exceeded 30 S cm⁻¹, in vivo measurements within the brain showed lower conductivity due to biological sequestration and other factors. However, even under these conditions, A5 demonstrated favorable resistance values and a clear current-voltage dependence, showcasing its applicability in bioelectronics.

### Electrofunctionalizing the Polymer of Formula (I)

Using the columnar injection technology gives essential control over the positioning and pattern of the soft electrode in the tissue, perhaps more so than the assembly controlled by the genetic expression of enzymes. To add modularity to the polymer of formula (I), e.g. A5, electrode-tissue interface, an auxiliary method that enables flexibility in the electrode surface area, functionality, and further protrusion for seamless extension into the tissue was developed. Co-injecting the aforementioned ETE-R derivatives, e.g. ETE-S, with e.g. A5 would position the soft electrode at the targeted site. Because of the concentration gradient and the electrostatic repulsion between the negatively charged ETE-S and the negatively charged A5, the former diffused from the formed A5 electrode. After a delay, electropolymerization using A5 as an electrode attached the ETE-S to the surface of A5 and intercalated within the A5 backbone.

In addition to increasing the surface area and tissue reach, different substituents I on the ETE-R derivatives would change the properties of the the final polymer electrode, as exemplified herein by e.g. ETE-S and the zwitterionic ETE-phosphatidylcholine (ETE-PC). Further, it may also be possible to have a mixture of different trimers, such as ETE, EEE or TET, with different R-groups, in order to tune the desired properties of the composition. The required high solubility of the injectable electrode imposes requirements on the nanoparticles forming the soft electrode, namely that they should be highly water-soluble and still be able to self-organize when injected into the tissue and thereafter bioresorbable without harming tissue. Thus, instead of redesigning the oligomers forming the polymer of formula (I), e.g. A5, this modular approach takes advantage of unique polymer properties and then adds soluble trimers in situ to customize the electrode-tissue interface.

As an example, polymer of formula (I), such as the PEDOT-S polymer A5 (I-1), forms the backbone of the electrode. A5 can be merged with trimers, such as ETE-PC-R (e.g. II-24, II-25, II-26 or II-45), forming an injectable pre-electrode solution. Once injected and subjected to electrofunctionalization, the properties of the electrode surface are primarily determined by the trimer-R. Loading the syringe sequentially with various polymer blends can produce distinct attributes along the electrode length. For instance, a combination of A5 and ETE-PC might result in an electrode with surface insulation, a mechanism rooted in ion-repelling membrane filter technology, where the change in electron energy in the core has a minimal impact on ions. Therefore, when the electrode is coated with ETE-PC cellular ion activation (bioelectricity) is substantially reduced. In contrast, when A5 is mixed with trimer-R, where R represents a redox mediator, such as TEMPO, ferrocene, acetosyringic acid or catechol, the resulting electrode segment contains redox mediators. This setup can be customized—selecting redox mediators matching targeted redox processes—to influence specific redox reactions in and around a tumor

The auxiliary A5 modular concept was evaluated in an in vitro agarose gel (0.5%) cast with Ringer's solution, which mimics brain tissue. A5 (20 mg mL⁻¹) was dissolved in ETE-S or ETE-PC (40 mg mL⁻¹), without the formation of any precipitate. The dark solution was injected into agarose using a Hamilton syringe where the polymer, i.e. A5, instantly formed an aggregate. The diffusion of the ETE-R derivatives from the polymer A5 injection track was monitored using UV light (365 nm). After 2 h, ETE-R diffused from the A5 at a distance approximately twice the thickness of the A5 hydrogel electrode. The A5 was then the connected electrode, and the ETE-Rs were electropolymerized at an applied bias of 1.5 V. This enabled electropolymerization, even with possible contact resistance. The applied bias may be further optimized, but it was selected for further use to have a high tolerance to variances in contact resistances during electropolymerization.

The thickness of the formed A5 electrode increased, confirming the successful polymerization of ETE-S. Further image analysis showed dendritic structures growing from the A5 core. The electropolymerized regions also displayed increased conductivity. With one contact on the A5 (and the other in the agarose) during electropolymerization, the comparably high resistance in agarose ensures a limited, constant current flow. Instead, by contacting both electrodes on the A5 during electropolymerization, the decreased resistance may be directly mapped as an increase in the current flow. The current increased by an order of magnitude during polymerization over a time course of 12 min. No change in the geometry, except for dendrite formation, of the A5 core was observed. The decreased resistance was explained by the polymerized ETE-S intercalating between the A5 nanoparticles. Although, electropolymerization was optically observed by A5 darkening when applying the voltage, no corresponding increase in current flow for the first 5 min could be registered. The initial incubation time could arise from one or more resistive bottlenecks that limit the current. Once these bottlenecks were removed by electropolymerization, a gradual current increase was observed as electropolymerization continued uniformly along the A5. Keeping both ends of the A5 contacted allowed for cyclic voltametric measurements which showed that electropolymerization increased the current 100 times.

A-ETE-Rs electropolymerized in agarose was used as a basis for evaluating the mechanical properties. The brain is very soft with shear modulus of about 0.5-1 kPa which has been challenging to match for conventional inorganic electrodes. Electropolymerized A5-ETE-PC closely mimicked brain tissue with a static shear modulus of 0.57±0.1 kPa. The A5-ETE-S was electropolymerized in an agarose gel incorporating live cells to evaluate biocompatibility. Lung adenocarcinoma cells were molded, the A549 cell line, into an agarose gel without and with contrast-enhancing DiI (lipophilic stain) cell labeling. After injection of the A5-ETE-S solution, one end of the polymer electrode was contacted, and the grounded counter electrode was kept in agarose (outside the A5). During electropolymerization, the newly formed dendritic structures of ETE-S(sodium salt of II-1) extended from the A5 (1-1) and achieved cell contact and, in some cases, embedded the cells, creating close contact with the cells without any observable detrimental effects such as loss of cell integrity. A close connection between the electrode and cells has been shown to be necessary for efficient and precise low voltage electrical stimulation-recording. A5 and ETE-R toxicity was also evaluated in a limiting-dilution assay, wherein neither A5 nor ETE-PC showed cell toxicity after 1 day exposure in up to 1 mg mL⁻¹ A5 or ETE-PC. On the other hand, ETE-S showed some toxicity at high concentrations. The toxicity study covered a wide range reaching up to more than 1000 times more ETE-R than were injected during in vivo experiments (around 200 μg vs. 400 ng), and a high dilution occurred when injecting into the tissue. For the quantities used in the in vivo setting, no cell toxicity from any of the compounds was observed. Thus, the conclusion of the in vitro experiments of the present inventive concept, is that A5 mediated electropolymerization enabled flexible surface modification, close contact with cells, and significantly decreased electrical resistance.

### Electropolymerization In Vivo

Transferring the above approach into an in vivo setting puts severe constraints on the experimental settings: 1) small diameter injection capillary to avoid blood vessel rupture; 2) A5, ETE-R, and A5-ETE-R all need to be highly soluble and biocompatible; 3) applied voltages and currents used for the electropolymerization need to be kept low to not damage the brain tissue; and 4) the procedure needs to be quick to avoid anesthesia related damage. The 30 μm diameter capillaries, precoated with 50 nm iridium, were used to inject an A5-ETE-S solution into the brain of anesthetized zebrafish. After injection, the ETE-S was left to diffuse into the tissue for 1 min. The coated capillary was then used as the biased electrode to establish seamless contact with the injected A5. Placing the counter electrode onto the zebrafish skin at the nostril allowed electropolymerization in the sedated fish with a low current, e.g. 1-3 μA, mimicking the agarose setup. The procedure, injection, and electropolymerization lasted about 10 min and after an additional 5-10 min, the fish were awake and displayed normal behavior. Typically, no fast darting or odd swim patterns were observed, nor were any difficulties with buoyancy and balance indicative of injury to the brain or stress behavior due to discomfort, e.g. pain, observed. The lack of adverse events shows that the minimally invasive approach was sound and well tolerated by the fish.

Histological staining of the A5-ETE-S containing sagittal brain sections (30 μm thick) showed close contact between cells and the polymer at the polymer-cell interface. An A5-ETE-S injection extending deep into the brain between corpus cerebelli (C) and optic tectum (OT) with ETE-S dendrites extending radially from the A5 reaching into the granular and molecular layers of C and superficial layer of OT could be imaged. As in the in vitro cell-agarose model, the A5-ETE-S wrapped around the neurons, and some were even entirely surrounded by the conductive polymer electrode without any visible damage to the cells. This highlights the benefits of a gel-like microstructural electrode that allows for the exchange of metabolites and ions through the electrode to sustain cell homeostasis.

Inflammatory Response to A5 Injections: Initial observations post-A5 injections noted an inflammatory response due to mechanical injection, which resolved within a few days. This response was attributed to oxidation of tissue resulting from the injection process.

Introduction of Auxiliary Modules (ETE-Rs): The study investigated the impact of incorporating ETE-Rs (electropolymerized trimers) with A5 on tissue integrity. This did not exacerbate tissue damage or inflammation.

Comparative Inflammatory Response with ETE-S and ETE-PC: Both A5-ETE-S(sulfonate functionality) and A5-ETE-PC (phosphatidylcholine) variants were tested. Initial significant inflammatory responses observed around the injection sites began to decrease significantly within 7-9 days for ETE-S and resolved completely within 3 days for ETE-PC, indicating a rapid healing process.

Impact of Electropolymerization Voltage: The process of electropolymerization/electrofunctionalisation, crucial for forming the soft conductive dendritic electrodes, was carefully evaluated to ensure it did not induce further oxidative stress or damage to the tissue. Results confirmed that the applied voltage for electropolymerization was safe and did not lead to additional tissue oxidation.

Minimally Invasive Methodology and Conductive Dendritic Electrodes: The study underscores the methodology's minimally invasive nature, suitable for installing soft conductive dendritic electrodes in brain tissue. The approach, combining A5 with electropolymerized trimers, did not cause additional tissue damage and was compatible with normal fish behavior, demonstrating its potential for neurological applications.

### A5-ETE-R Electrical Properties in Zebrafish Brains

The conductivity of A5 was improved by ETE-R functionalization. For fish that underwent electropolymerization, more than 10 times higher currents (resistances of 5-10 MΩ) were observed under the same applied bias for e.g. both ETE-S and ETE-PC. It was also possible to map conductivity over long electrode distances, allowing for the deduction of a value on the conductivity. Adjusting to the increased diameter of the conductive polymer upon electropolymerization, it was estimated that the conductivity was around 3 S cm⁻¹ for both A5-ETE-S and A5-ETE-PC. This is 2 to 3 orders higher than most tissues (<10⁻² S cm⁻¹). Some fish were also left to swim around with the conductive polymer in the brain for 7 days, showing normal fish behavior. For A5-ETE-S and A5-ETE-PC, distinct polymers were clearly observed in the brains of two out of three fish but with lower conductivities than in the one-day experiments. The A5-ETE-PC showed a linear voltage-dependent current in the low nA regime (resistance around 1 G (2), thereby presenting a polymer still exhibiting long-distance conductivity. Interestingly, the higher 7-day conductivity of A5-ETE-PC indicates that this modification to the polymer rendered it more stable, thus different trimers gives different properties.

Methodology Overview: The disclosed methodology involves injecting a mixture of A5 and ETE-S into zebrafish brains, followed by electropolymerization. The process results in the formation of soft electrodes within the brain tissue, which are then analyzed for their ability to stimulate specific brain regions and alter neuronal activity.

Experimental Procedure: Adult zebrafish of the Casper mutant variety, genetically modified to express the GCaMP6f calcium indicator, were used. This allowed for the visualization of action potentials as increases in green fluorescence, providing a non-invasive method to track neural activity. The presence of A5-ETE-S electrodes was crucial for transmitting electrical pulses and inducing neuronal firing over long distances within the brain.

Spatial Specificity and Toxicity Testing: The experiments demonstrated that the conductive polymer could target specific brain regions without causing acute toxicity to the cells. The addition of PTZ (a GABAA receptor antagonist) indicated the ability to stimulate regions not directly adjacent to the electrodes, underscoring the method's potential for precise neural stimulation.

External Contacting Method: A novel approach allowed for the external contacting of the polymer electrodes within the brain. By leaving a portion of the microcapillary used for injection in place, it was possible to establish external contact with the soft electrodes, facilitating brain activity alteration without direct intervention on the brain tissue. This technique presents a significant advancement for clinical applications, offering a method to interact with internal bioelectronics non-invasively.

Functional and Transient Bioelectronics: The disclosed bioelectronics are not only functional but also well-tolerated within the brain, offering a promising avenue for transient bioelectronic therapies. The use of thiophene oligomers (A5) to form highly water-dispersed nanoparticles enables the creation of stable soft electrodes upon interaction with endogenous ions, eliminating the need for specific triggers for electrode formation.

The present disclosure thus relates to functional and well-tolerated organic bioelectronics in the brain. To meet the demand for transient bioelectronic therapies, implantation was performed using a minimally invasive injection technique, and the formed structures were bioresorbable. The latter is a desired property in, for example, electrotherapeutic cancer treatments, making revision surgery obsolete. This was made possible by using polymers of formula (I), such as thiophene oligomers (A5), that form nanoparticles. These nanoparticles are highly water-dispersed, which makes it possible to have them in high concentrations in a solution without aggregation. However, by injecting them into the tissue and following interaction with endogenous ions, a stable soft electrode is formed. Thus, no specific triggers are necessary. It was also possible to ion-match the nanoparticle solution with endogenous ion strengths in quite different tissues, as shown by establishing conductive structures in, for example, the zebrafish caudal fin and brain. This makes the present disclosure general to several tissues and cross-species. Also, because the nanoparticles comprise oligomers that are the size of conventional drugs; they are bioresorbable.

The unique properties of the polymers of formula (I), such as A5, to generate functional flexibility at the electrode-tissue interface, enabled developing an auxiliary modular methodology. This modular approach takes advantage of said properties and then adds soluble trimers, i.e., one or more compound of formula (II) or formula (III) that attach to the polymer in situ to customize the electrode-tissue interface. The electropolymerization of the trimers with low oxidation potential in situ increased conductivity, formed a close connection to cells and established functional group modifications to the polymer electrodes. This was demonstrated using for example trimers ETE-S and ETE-PC, where the latter had higher long-term stability and less toxicity. Furthermore, it has been shown that electropolymerization did not cause additional oxidation damage to brain tissue. This modular approach opens up for the one or more compound of formula (II) or formula (III) having different functional groups and potentially other trimers with low oxidation potential.

Despite the small zebrafish brain, the critical challenge of contacting soft neural electrodes to allow efficient external interaction was solved. With external contact, neuronal signaling was modulated in live brain slices, excised from fish with implanted bioelectronics, by applying electrical pulses.

The methodologies and workflows presented here are general and not confined to zebrafish, and the procedures would be more straightforward with larger brains, e.g. rodents and primates, especially external connections.

In summary, the present disclosure relates to in vivo assembled, fully integrated, bioresorbable electronics within nervous systems and other tissues that can be used for nonchronic treatments.

### Example 1

### Evaluation of the A5-ETE-S Agarose Mold

A solution of A5 (20 mg mL⁻¹) and ETE-S(40 mg mL⁻¹) in H₂O was injected into an agarose mold (0.5% agarose in Ringer solution). Diffusion of the ETE-S was monitored using a UV lamp at 365 nm. After 2 h, one of the Au-coated W-electrodes were connected to the A5 aggregate (and one in the agarose mold to electropolymerize ETE-S to get 100% coverage. ETE-S was electropolymerized using 1.5 V (Keithley 2612B). The agarose was imaged using brightfield microscopy (10× and 40× objective).

The conductivity of the A5 and A5-ETE-S in agarose was measured using a two-terminal setup in which 25 μm Au-coated tungsten microprobes (Signatone, Gilroy, CA) were connected to the polymer embedded in the agarose. By sweeping an applied electric potential and registering the resulting current over different distances, conductivity can be estimated using the transmission line model.

### MTT Assay

The MTT cell viability assay was conducted to determine in vitro toxicity of A5, ETE-S and ETE-PC. Briefly, HLF-1 (2×10⁴ cells well-1) were plated in a 96-well flat-bottom microplate and grown for 24 h. Cells were treated with A5, ETE-S, or ETE-PC (0-1000 μg ml⁻¹) for 24 h. Post-treatment, 200 μL of MTT (0.5 mg mL⁻¹) was added into each well and incubated at 37° C. for 4 h. After incubation, 200 μL of isopropanol was added to dissolve formazan crystals. The optical density of formazan solution, as a measure of live cells, was obtained using a microplate reader at 570 nm (Spark Cyto, Tecan). The formazan signal (live cell count) was normalized to the control samples not exposed to A5 or ETE-Rs. 3 biological and 3 technical replicates were used for each setting. Each well contained up to 200 μg of our compound (1 mg mL⁻¹, 200 μL) which can be compared to a typical in vivo injection of 400 ng (40 mg mL⁻¹, 10 nL).

### In Vivo-Tail Fin Assay

Before microinjection, fish were anesthetized with tricaine (ethyl 3-aminobenzoate methanesulfonate; 0.2 mg mL⁻¹) until opercular movements had ceased and the fish did not respond to vibrations caused by tapping close to the tricaine container. An anesthetized fish was placed on its side on a plate filled with 1% agarose (Agarose, LE, Analytical Grade, Promega Corporation) in E3 medium that had been allowed to solidify. A piece of moist tissue paper was placed over the fish to keep the body from drying but still exposing the caudal fin. The plate was then transferred to the microinjection setup, and a glass capillary with a 30 μm diameter bevelled tip (cat. No. BM100T-15. Bevelled, straight, shortened+firepolished ends from Biomedical-Instruments GMBH) filled with polymer solution was used to inject the inter-rays of the caudal fin. The total injection volume per inter-ray was estimated to be in the 100 nL range. After injection, the fish were revived directly by flushing the gills with fresh aquarium water and transferred to a post-op aquarium for observation.

In Vivo-Brain. Surgery and Microinjection

Before surgery and microinjection, fish were anesthetized with tricaine (ethyl 3-aminobenzoate methanesulfonate; 0.2 mg mL⁻¹) until opercular movements had ceased and the fish did not respond to tail pinching. For surgery, the anesthetized fish was placed in a mold made of moist tissue paper for stabilization. Then, a small hole was made in the parietal bone just above the corpus cerebelli and immediately left of the midline with the tip of a 30G needle. The fish was then transferred, in its tissue paper mold, to the microinjection setup, and a capillary filled with polymer solution (see below) with a 30 μm diameter bevelled tip (cat. No. BM100T-15. Bevelled, straight, shortened+firepolished ends from Biomedical-Instruments GMBH) was inserted through the hole in the skull roof to a depth of 700 μm. Then, three injections were made: one each at 700 μm, 500 μm, and 300 μm depths. The total injection volume was estimated to be 10 nL. After injection, fish were either revived directly by flushing the gills with fresh aquarium water and transferred to a post-op aquarium for observation or subjected to electropolymerization.

In Vivo—Brain. Electropolymerization

When the polymer injection was followed by electropolymerization, a counter electrode was placed on the skin at one of the nostrils, and an iridium coated polymer-containing capillary served as the electrode. Injections were performed as described above; after the injection, the capillary was left in place in the brain. After 1 min—allowing the polymer solution to diffuse—the injected polymer was electropolymerized by applying 1.5 V (current around 1-3 μA) over the electrodes for 5 min using a Keithley sourcemeter (Keithley Instruments). One side of the 30 μm diameter glass injection capillary (cat. No. BM100T-15. Bevelled, straight, shortened+firepolished ends from Biomedical-Instruments GMBH) was pre-coated with 50 nm Ir in a Quorum sputterer (QT 150, Quorum technologies) resulting in a conductive capillary with maintained backside optical access to verify liquid levels before injection.

When experiments were of longer out-of-water duration than 10 min, the fish was intubated for superfusion of the gill chambers with aerated aquarium water containing 0.1 mg mL⁻¹ tricaine, using a Peri-Star Pro peristaltic pump (World Precision Instruments). Initially, we experimented with diffusion and electropolymerization durations longer than 5 min each. This did improve polymer spread and polymerization, but did also affect the fish more negatively.

### Polymer Formulations for Microinjections

The following polymer formulations were used for microinjections into the brain (all dissolved in Millipore water): A5 (20 mg mL⁻¹); A5 (20 mg mL⁻¹)+ETE-S(40 mg mL⁻¹); A5 (20 mg mL⁻¹)+ETE-PC (40 mg mL⁻¹).

### Post-Experiment Tissue Processing

After polymer injection, with or without electropolymerization, the fish was allowed to recover as described above and then transferred to aquaria for different post-injection survival times. Fish were sacrificed for histological examination or conductivity measurements after 1 h, 2 h, 3 h, 4 h, 1 day, 2 days, 3 days, 7 days, 8 days, or 9 days. The fish were euthanized by immersion in ice-cold water for 10 min and then decapitated. The brains were either excised directly without fixation for freezing on dry ice in TissueTek OCT (Brand) or processed immediately for redox staining (see below), or the skull roof was opened and the head (jaws removed) fixed overnight in 4% paraformaldehyde in 0.1 M phosphate buffer.

Fresh frozen brains were cryosectioned in the sagittal plane (20-50 μm section thickness, depending on subsequent processing) in a Cryostar NX70 cryostat. Sections were mounted on Superfrost Gold microscope slides for microscopy, or on interdigitated gold electrodes for conductivity measurements.

Paraformaldehyde-fixed brains were excised from the skull, rinsed in phosphate-buffered saline (PBS), cryoprotected in PBS with 25% (w/v) sucrose, and frozen on dry ice in TissueTek OCT. The brains were cryosectioned in the sagittal plane (30-50 μm section thickness, dependent on subsequent processing). Sections were mounted on Superfrost Gold slides for further processing.

### Electrical Measurements

Brain sections with polymer were placed on interdigitated Au electrodes connected to a Keithley sourcemeter. Two of the interdigitated electrodes were contacted using external microelectrodes. An applied voltage was swept and the resulting current was registered. This was repeated for all interdigitated electrode leads covering the conductive polymer. Distance between adjacent electrodes was 15 μm, and the width was 2.5 mm.

### Injection and Brain Slice Preparation

A mixture of A5 and ETE-S was microinjected and electropolymerized in the brain, as described above, in adult zebrafish Casper mutants (Tg(elav3:GCaMP6f)). For these experiments, the capillary was cut immediately above the skull roof and left with the tip superficially in the brain. One day post-injection, the fish were euthanized by immersion in ice-cold aquarium water and decapitated. The brains were rapidly dissected and embedded in 3% low melting point agarose dissolved in Zebrafish normal Ringer solution. The blocks were cooled on a metal plate and transferred to NMDG cutting solution on ice, trimmed and mounted for vibratome sectioning. 300 and 400 μm sagittal sections containing the capillary tip and the polymer electrode in the tissue were cut from each brain, transferred to HEPES recovery solution and allowed to reach room temperature (ca 24° C.). The vibratome section was then transferred to artificial (zebrafish) cerebrospinal fluid (aCSF), inspected for GFP positivity, and prepared for electrical stimulation and Ca²⁺-imaging.

### Electrical Stimulation in Brain Slices

10 μm tungsten microelectrodes (Signatone, Gilroy CA) were allowed to contact the A5 in the brain slices, either directly or by contacting the injection capillary which in turn was contacted to the A5. A Grass S48 stimulator (Astro Med) was used to supply the stimulating square voltage pulses with the following settings: 2 trains per second, 200 ms train duration, 20 pulses per second, and 2 ms pulse duration. The magnitude of the voltage pulses as dialed in on the Grass stimulator ranged between 6-14 V. Large losses, including contact resistances, stray currents in buffers surrounding the tissue slices, poor impedance matching, and others motivate the need for a reasonably high stimulating voltage. Extensive bubble formation around the electrodes, which would be the case at high input powers, was not observed.

### 3D Imaging

Samples were imaged in a chamber filled with DBE. The cleared brain slice embedded in agarose was imaged on an Ultra Microscope II (LaVision Biotec) equipped with an sCMOS camera (Andor Neo, model 5.5-CL3) and 4× objective lenses (LaVision LVMI-Fluor 4×/0.3). Two laser configurations (488 nm and 640 nm) were used with the following emission filters: 525/50 for endogenous background (blood vessels) and visualization of ETEs trimer and 680/30 nm for visualization of neurons (Neurotrace 640/660). Stacks were acquired with ImspectorPro64 (LaVision Biotec) using 3 μm z-steps to acquire the volume in 3D. This image stack was stitched to visualize the brain slice in 3D with Arivis Vision 4D 3.5.0 (Arivis AG). Rendered movie was compiled in Final Cut Pro 10.4.3 (Apple Inc.).

### Mechanical Measurements

0.6% agarose molded in Ringer buffer was injected with 3 μl A5 [20 mg ml⁻¹]+ETE-PC [40 mg ml⁻¹] using a Hamilton syringe. The A5-ETE-PC was electropolymerized at 1.75V for 20 min. Cross-sections were cut and placed in a Biomomentum Mach-1 mechanical tester. The test was performed in indentation mode, with a 0.5 mm diameter spherical indenter at a speed of 0.01 mm/s. Indenter depth: 0.15, 0.3 and 0.45 mm. The test profile consisted of the following steps:
Contact (0.1 gf)
10 min wait to recover from contact
3 stress-relaxations
3 sinusoidal test at 0.1, 1 and 4 Hz, respectively
   The agarose and A5-ETE-PC were indistinguishable at 0.1 Hz. At 1 Hz, the A5-ETE-PC had a lower modulus. Looking at the component of the Modulus, the agarose showed a higher elastic response, whereas the A5-ETE-PC may have become more viscous.

### Example 2: Biocompatible 3D Flexible Electrode for Electrotherapy of Glioblastoma Enzymatic Polymerization of ETE-PC

A solution of ETE-PC (500 μg/ml) was prepared in DPBS (Thermo Fischer, Gibco, Cat no. 14190-250) and mixed with different concentrations of hydrogen peroxide (H₂O₂) (0.001-0.01%) and horseradish peroxidase (HRP) (Merck, Sigma Aldrich, P8375-25KU) (5 U/ml in DPBS) was added. The polymerization was examined by the change of colour of the ETE-PC solution from slightly yellow to black and taking absorbance at 350 nm and 780 nm to detect polymerized and unpolymerized ETE-PC. The absorbance maxima of polymerized and unpolymerized ETE-PC were determined by a wavelength scan using UV-Vis spectrophotometry by Spark Cyto (Tecan) multi-mode plate reader.

The wavelength scan by UV-Vis spectroscopy determined the absorbance maxima of unpolymerized and polymerized ETE-PC to be 350 and 780 nm respectively. The enzymatic polymerization results showed that HRP can successfully polymerize ETE-PC in presence of H₂O₂ as exhibited by change in color of ETE-PC solution. Further, the concentration of H₂O₂ required for HRP mediated polymerization of ETE-PC is as low as 0.001%. The absorbance of polymerized vs unpolymerized ETE-PC has been represented in the form of line graph showing decrease in absorbance of unpolymerized ETE-PC while increase in polymerized ETE-PC. Further, the polymerization of ETE-PC by HRP got saturated at 0.002% of H₂O₂. These data suggest that HRP can effectively polymerize ETE-PC in presence of very low concentration of H₂O₂. Tumor cells secrete high amount of H₂O₂ as compared to normal cells. This data suggests that HRP may be used to polymerize ETE-PC in the tumor microenvironment by using H₂O₂ secreted by cancer cells which create possibility of manipulating tumor microenvironment for electrotherapy of cancer.

### Enzymatic Polymerization of ETE-PC in the Presence of Cancer Cells

The cancer cell mediated enzymatic polymerization of ETE-PC using 2D and 3D glioblastoma models were examined.

For the 2D model, U87 glioblastoma cells were seeded in a 96 well plate at a density of 10,000 cells per well in phenol red-free (PR-free) complete DMEM (Thermo Fisher, Gibco, Cat no. 21063045) and maintained in a CO₂ incubator for 24 hrs at 37° C. After 24 hrs, cells were treated either with HRP (5U/ml in DPBS) and ETE-PC (100 μg/ml in PR-free DMEM) alone or in combination. Cells were incubated for 72 hrs, and ETE-PC polymerization was observed by bright field microscopy (10× objective) and UV-Vis spectrophotometry (350 nm and 780 nm by Spark Cyto (Tecan) multi-mode plate reader) at 24, 48, and 72 hrs interval.

The cells treated with ETE-PC/HRP mix demonstrated black deposits of polymerized ETE-PC at 24 hrs which became increasingly darker at 48 and 72 hrs. Cells treated with only HRP or ETE-PC did not show any polymerization. The UV-Vis spectroscopy results validated these observations where the absorbance of unpolymerized ETE-PC was decreased while that of polymerized one increased as the time progressed.

In the case of the 3D model, to mimic brain tissue, we used low-concentration agarose (0.5% [Agarose, low gelling temperature, Sigma Aldrich, Cat no. A9414-25G] in PR free DMEM) gel cast embedded with U87 cell or spheroids. Briefly, 100 μl of U87 cells or spheroid suspension in PR-free DMEM were mixed with 1% agarose (100 μl in PR-free DMEM) to get 0.5% agarose concentration and added into wells of 96 well plate. Cells were kept in CO₂ incubator at 37° C. for 24 to normalize. Cells/spheroids were then treated with ETE-PC (100 μg/ml in PR-free DMEM) either alone or in the presence of HRP (5U/ml in PBS) and incubated for 72 hrs. Post 72 hrs, images were taken using bright field microscopy (10× objective).

Both cells and spheroids demonstrated polymerization of ETE-PC in presence of HRP as observed by black color deposits around the cells/spheroid as well as whole well. These results suggest that once injected ETE-PC and HRP can diffuse into the tumor tissue and polymerize within it with the help of H₂O₂ present in tumor microenvironment.

### Electrofunctionalization of ETE-PC on A5 and Preparation of A5/ETE-PC 3D Flexible Electrode In Agarose

To prepare A5/ETE-PC 3D flexible electrode, 200 μl of 0.5% agarose (Agarose, low gelling temperature, Sigma Aldrich, Cat no. A9414-25G) was added to wells of 8 well chamber slide. Once the agarose gelled, a solution of 20 μl solution of A5/ETE-PC (20 mg/ml A5 and 40 mg/ml ETE-PC in PR-free DMEM) was added to one side of the well, and ETE-PC was left to diffuse laterally into the agarose for 2 hours. After 2 hours, A5 was electrofunctionalized with ETE-PC to form flexible electrodes by applying a bias of 2V vs Au counter electrode (Keithley sourcemeter 2612B, Keithley Instruments) for 30 min. The electrode formation was examined by bright field microscopy (10× objective).

After application of A5/ETE-PC mix, A5 quickly turned in to a thick gel while ETE-PC started diffusing into agarose. High magnification images after electrofunctionaliztion show dendritic structure of ETE-PC branching out of A5 confirming formation A5/ETE-PC flexible electrode.

Post electrode formation, impedance was measured by performing Electrochemical impedance spectroscopy (EIS) using Autolab PGSTAT204 potentiostat (Metrohm) for the frequency range of 1 Hz to 100 KHz in increments of 10 Hz. Impedance measurements on agarose gel without flexible electrode as control.

The EIS studies demonstrated that use of flexible electrode significantly decreased the impedance in agarose based electrical system suggesting that A5/ETE-PC flexible electrode can enhance conductivity of electrical system. Further, A5/ETE-PC electrode was prepared around U87 cell and spheroids for conducting in vitro experiments. To do so, U87 cells were seeded in wells of chamber slide at a density of 20,000 cells per well and maintained in DMEM media in incubator for 24 hrs at 37° C. and 5% CO₂ conditions. After 24 hrs, 0.5% agarose (200 μl) was added on to the cells to make a layer and A5/ETE-PC mix was applied from one side of well. ETE-PC was allowed to diffuse for 2 hrs and electrofunctionalize to prepare the electrode. Cells were stained with calcein-AM for identification. In case of spheroids, U87 spheroids prepared by hanging drop method were mixed with 0.5% agarose and added to well of chamber slide to make a 3D model of spheroid embedded agarose. The A5/ETE-PC electrodes were prepared as described above. The result show successful synthesis of dendritic soft electrodes branching from A5 surrounding the cells as well as spheroids. No detrimental change was observed in morphology of cells as well as spheroids suggesting biocompatibility of electrodes. These results provided us a functional 3D cancer model for assessing the ability of A5/ETE-PC electrode in electrotherapy of cancer.

### Irreversible Electroporation of Cancer Cells by A5/ETE-PC Electrode

The efficacy of A5/ETE-PC flexible electrode in electrotherapy of cancer was examined by performing irreversible electroporation (IRE) in 3D in vitro models (cell and spheroid) of glioblastoma in agarose (Agarose, low gelling temperature, Sigma Aldrich, Cat no. A9414-25G) mold. Briefly, 100 μl of U87 cells or spheroid suspension in PR-free DMEM were mixed with 1% agarose (100 μl in PR-free DMEM) to get 0.5% agarose concentration and added into chambers of 8 well chamber slide (μ-Slide 8 well^{high} ibiTreat, ibidi). Cells were kept in CO₂ incubator at 37° C. for 24 hrs to normalize. After 24 hrs, the A5/ETE-PC electrode was prepared in cells/spheroids containing agarose mold as described above.

To perform IRE, high voltage pulse electric fields were applied (MicroPulser, Bio-Rad) to cells/spheroids using A5/ETE-PC electrode. Specifically, cells were treated with a series of pulse electric fields (200-800 V/cm) by applying 3 sets of 1 ms long pulses with 50 pulses in each round. For comparison, similar treatment was performed with conventional Au needle-shaped electrodes with identical settings. Sample with no treatment was used as control. After treatment, 100 μl live cell staining dye calcein-AM (Thermo Fisher, Invitrogen, Cat no. C3100MP) (2 μM in PR-free DMEM) was added to chambers and incubated for 30 min followed by examination of live cells using fluorescence microscopy.

The result demonstrated that IRE using Au electrodes induced significant cell death at 600 and 800 V as determined by decrease in green fluorescence. However, IRE using A5/ETE-PC electrode exhibited almost complete killing effect from 200 V itself in both cell and spheroid based glioblastoma models. This shows a significant increase in IRE efficacy when using A5/ETE-PC electrode. Conventional solid needle electrode covering small surrounding them leading to requirement of higher voltage to cover large amount of tissue which may result in side effects. In contrast, due to its flexibility and extensive reach of A5/ETE-PC electrode covers more area and hence may require low intensity electric field to perform effective IRE. These results open the possibility of A5/ETE-PC electrode for significantly more effective electrotherapy through IRE. However, the results need to be replicated in vivo first.

### Cytotoxicity Assessment of ETE-PC in Normal Lung Fibroblast

Study of the in vitro toxicity of ETE-PC in normal human lung fibroblast (HLF-1) cells. Briefly, HLF-1 cells were seeded in 96 well plate at a density of 2×10⁴ cell per well and grown for 24 hrs. Later cells were treated with various concentrations of ETE-PC (5-1000 μg/ml) for 24 hrs. Post treatment, 200 μl live cell staining dye calcein-AM (Thermo Fisher, Invitrogen, Cat no. C3100MP) (2 μM in PR free DMEM) was added to wells and incubated for 30 min followed by examination of live cells using fluorescence cell imaging (Spark Cyto cell imager, Tecan).

The results demonstrated no change the calcein-AM fluorescence (green) in cells treated with ETE-PC even at a high concentration of 1000 μg/ml as compared to control. No detrimental change in morphology of HLF-1 cells was observed in phase contrast images. This suggest that ETE-PC is highly biocompatible to normal cell making it suitable for therapeutic use.

### Preparation of A5/ETE-PC Flexible Electrodes in U87 Tumor In Vivo

U87 tumors on CAM (chicken chorioallantoic membrane) were injected with a mixture of A5/ETE-PC (20/40 mg/ml) and initially ETE-PC was electrofunctionalized on A5 by applying a 1.2 V bias for 2.5 min. ETE-PC was then allowed to diffuse in tumor tissue for 5 minute and a second electrofunctionalization was performed by applying 3V for 15 min. Post electrofunctionalization, tumors were harvested and fixed in 4% paraformaldehyde for 24 hrs at 4° C. and washed with PBS followed by incubation in 30% sucrose solution for cryoprotection. The tissues were frozen on dry ice in TissueTek OCT (Fisher scientific: epredia Neg-50). Frozen tumor and liver sections were cryosectioned (10-50 μm thickness) using Cryostar NX70 cryostat and mounted on Superfrost Gold microscope slides for microscopy. Images were taken using bright field microscopy (using 4×, 10× and 20× objectives). During the experiment, EIS was performed on tumor before and after preparation of flexible electrodes in the tumor.

The EIS data demonstrated a significant drop in impedance in tumor post injection an electrofunctionalization of A5/ETE-PC in tumor. This indicates the successful preparation of A5/ETE-PC electrode in tumor and is ability to significantly enhance conductivity within tumor tissue. The microscopic image of tumor section proved the assembly of flexible electrode in tumor. Nicely formed dendritic structure growing from A5 core were observed depicting successful electrofunctionalization of ETE-PC. However, fluorescence imaging exhibited presence of unpolymerized ETE-PC as revealed by green fluorescence of trimer. This suggests that while significant electrofunctionalization of ETE-PC occurred, it is not optimum. Hence, electrofunctionalization may be performed using different conditions to optimize the preparation of flexible electrode in tumor.

### Electrode Formation and Distribution in Tissues, Tumors, and Around Cancer Cells

Preliminary studies on blanket formation and tissue penetration were conducted using a mouse brain model. In this experiment, a cavity was created, the nanoparticle solution was applied, and as anticipated, a blanket was formed with dendrites extending into the tissue. To connect this to treatment, designed to simulate a post-surgery scenario, GBM U87 cells and spheroids were embedded in agarose gel. A5/ETE-PC was added on top and diffused into the agarose; subsequent electrofunctionalization generated dendrites. These dendritic structures created close contacts and embedded cells and spheroids that manifested in an increased efficacy during IRE compared with the gold electrode.

It has been further demonstrated galvanotaxis, where cells actively move toward the electrode layer in response to an electric field, which might be advantageous for the treatment to halt invasive cells, bring them closer to the electrode, and eliminate them.

The term “abscopal”—meaning “away from target”—was coined after documenting the remission of tumors outside the radiation field in patients with metastatic disease. Since then, researchers have published almost 50 case reports about the abscopal effect on various cancers after radiotherapy. This represents quite a few cases, considering that approximately 50-60% of cancer patients receive radiation at some point during their disease. The abscopal effect is caused by an immune response against tumors that are not treated directly. However, the rarity of the abscopal effect underscores the difficulties that need to be overcome to elicit a significant immune response. Five key events have been linked to effective priming of the T cells: (I) release of TAAs, (II) release of damage-associated molecular patterns (DAMPs), (III) uptake and processing of TAAs by antigen-presenting cells (APCs), (IV) antigen presentation by APCs to naïve T cells, and finally, (V) activation and proliferation of cancer-specific CD8+ T cells, reversing immunosuppression in the tumor environment. Notably, mild infections were observed more frequently in patients who developed an abscopal effect. The role of infection-driven inflammation as an abscopal effect-promoting factor is exciting and can be further explored. We believe that modular electrotherapy-variations of pulse sequences combined with redox modalities-will predictably unleash the abscopal effect.

IRE has shown promising results in cancer therapy, but its effectiveness is limited to tumor cells within proximity of 60-100 μm to the electrode. To overcome this limitation, we propose integrating redox modalities into soft electrodes, which can be implanted into and around tumors to target specific redox reactions. IRE and redox modulation represent two distinct approaches to cancer therapy. Both methods have shown some potential in activating the immune system to combat cancer cells, with synergies observed in the activation of immune cells, antigen presentation, and cytokine and chemokine production, meeting all five key elements. IRE releases DAMPs and TAAs that stimulate the activation of immune cells, such as macrophages (e.g., microglia), dendritic cells (DCs), and T cells, which can be done with controlled electroporation, releasing the cellular content over an extended period and thus giving the APCs time to engulf and present antigens. Some of the released TAAs are unstable (e.g., mRNA) and rapidly eliminated; however, electroporation could facilitate both the release from cancer cells and uptake by the APCs, in a lateral transfer between cells. Moreover, IRE enhances the activity of NK cells, which can recognize and kill tumor cells without prior sensitization. Additionally, redox reactions modulate the intracellular redox environment and tumor microenvironment, indirectly activating NK cells by the cytokines IL2, IL12, and IL15 released from immune cells. Other redox targets include key molecular components such as glutathione (GSH) and redox-sensitive cysteine residues in target proteins. These components play a crucial role in maintaining redox homeostasis within cells. They can impact critical signaling pathways responsible for cancer cell proliferation, survival, and metastasis, such as the nuclear factor kappa B (NF-κB) and STAT3 pathways. By targeting the increased ROS production in cancer cells, which are more susceptible to oxidative stress than healthy cells, we can undermine the natural defense mechanism against ROS and RNS by reducing GSH levels. Another approach is to target tumor cell metabolism by depleting essential amino acids, such as cysteine, tyrosine, and lysine. Redox modulation in the tumor microenvironment can also influence the polarization state of tumor-associated macrophages (e.g., microglia), shifting them from the pro-tumorigenic M2 phenotype to the anti-tumorigenic M1 phenotype (this oversimplified model is used here for clarity), enhancing the antitumor immune response, and suppressing tumor growth. These effects complement IRE-induced DAMP release, demonstrating the synergies between IRE and redox reactions in activating the immune system. Another potential target is the tumor vasculature, which is critical for tumor growth and metastasis. Supplementing IRE with redox-targeting modalities can enhance the efficacy of cancer treatments by combining their strengths: targeting the intracellular redox environment, tumor microenvironment, and immune system to achieve a more effective and targeted cancer therapy.

### Example 3: In Situ Assembly of a Bioresorbable Injectable Cardiac Stimulator

### Background

In cardiac arrests or dangerous arrhythmias, the general approach to restoring heartbeats typically encompasses administering electric stimuli through the utilization of a defibrillator, the implantation of a pacemaker, or both. A compact, bioresorbable injectable cardiac stimulator (BICS) the size of a pen could serve as a less invasive and lighter alternative to traditional defibrillators and pacemakers, which necessitate open surgery for implantation. Envisioned for short-term application, especially in hard-to-reach rural areas, BICS aims to circumvent the challenges of transporting bulky, heavy equipment or conducting open surgeries. It is particularly suited for temporary heart stimulation in remote clinical scenarios until the patient can be transferred to a facility equipped for permanent implantation. Utilizing imaging guidance, such as ultrasound, ensures precise placement similar to the interventional technique of pericardiocentesis. However, in urgent life-threatening situations, such as in war zones or remote regions where advanced imaging tools might not be available, the device can be guided into position using anatomical landmarks.

The conductivity of the implanted hydrogel was reported to be slightly below 14 mS/cm, which was approximately twice that of the surrounding tissue (6 mS/cm). Blood conductivity is reported to range between 10-20 mS/cm, which aligns with the conductivity of the hydrogel. Most prior art in vivo injectable conductive hydrogels are employed in a passive capacity, e.g., cardiac patches, or reported without showing external connectivity, and they typically exhibit conductivities in the low mScm⁻¹. However, no reported hydrogel in the prior art has been presented to work for heart stimulation. In addition to injecting enough energy for cardiac stimulation, developing injectable electrodes for cardiac applications faces several challenges. These include the need for the formed electrode to firmly adhere to the beating heart's surface without impairing its natural synchronized movements, matching the elasticity and stiffness of the cardiac tissue, irrespective of the gross placement of the polymer on the heart. The pro-electrode formulation should be administered through thin capillaries for minimal invasiveness. Thus, the formulation must be highly soluble for injection yet prone to aggregation into a conductive structure in vivo to adhere to the beating heart and to form an external connection. These critical adherents-softness and solubility-aggregation dualities are critical and challenging material design criteria. Additionally, after serving its purpose, the hydrogel should be bioresorbable and non-toxic during this process.

Given the intricate complexity of developing BICS, criteria that are challenging to assess solely through in vitro methods. Zebrafish (Danio rerio) offer a viable model for studying the effects and interactions of various materials in a living organism. Zebrafish are increasingly used in replicating human cardiac pathologies, including arrhythmias, because they have a heart rate closer to humans (120 bpm for zebrafish, 60 bpm for humans) than mice (600 bpm), potentially making them a better model in this context. Although there are differences between zebrafish and human hearts, such as zebrafish having a two-chambered heart (one atrium and one ventricle) compared to the four-chambered hearts in humans, key aspects of cardiac electrophysiology are conserved across both species. A model with close anatomic resemblance with the human heart is the chicken embryo heart model, a 3R in vivo model that offer the advantages of an explorative model.

Thus, 8-(2-(2,5-bis(2,3-dihydrothieno[3,4-b][1,4]dioxin-5-yl)thiophen-3-yl)ethoxy)-1-(tri-methylammonio)octane-4-sulfonate (ETE-BuSA), a zwitterionic thiophene trimer was considered suitable for in vivo applications. ETE-BuSA mixed with poly(3,4-ethylenedioxy-thiophene) butoxy-1-sulfonate (PEDOT-S, A5) formed a highly water-soluble mixture, denoted proBICS. Injected into the pericardial cavity using a small-diameter capillary proBICS self-organize into a mixed ion-electron conducting hydrogel-BICS around the heart and was used to stimulate the heart of zebrafish and chicken embryos. Furthermore, the conductive hydrogel is extended out of the pericardial cavity and placed on the skin as an external contact point to relay an external stimulus. The conductive hydrogel, designed to be temporary, leaves no damage from the electrode. Additionally, animals with this implant showed no behavioral changes during and after its bioresorption, and their offspring exhibited neither developmental nor behavioral issues.

### Synthesis of ETE-BuSA

The synthesis of ETE-BuSA begins by coupling 2-(2,5-dibromothiophen-3-yl)ethan-1-ol with EDOT pinacol boronate ester. This step employed the palladium catalyst, PEPPSI-IPr, and resulted in the formation of ETE-OH with a 48% yield; this step was later repeated on a larger scale (11 g), giving an improved yield of 74%, showing a nice scalability. Next, ETE-OH underwent alkylation using dibromobutane in the presence of a tetrabutylammonium bromide (TBAB) catalyst, producing ETE-BuBr with a 75% yield. The synthesis continued by deprotonating 1,4-butane sultone using n-butyllithium (n-BuLi) at −78° C. in anhydrous tetrahydrofuran (THF) and quenching with ETE-BuBr, serving as the electrophile, leading to the formation of ETE-BuSultone. Finally, ring-opening of ETE-BuSultone with trimethylamine yielded the target molecule, ETE-BuSA. After dehydration, ETE-BuSA powder was easy to handle and could be stored in a regular freezer.

Synthesis of 2-(2,5-bis(2,3-dihydrothieno[3,4-b][1,4]dioxin-5-yl)thiophen-3-yl)ethan-1-ol (ETE-OH) (II-10). In a two-neck flask filled with dry THF (50 mL) under a nitrogen atmosphere, 2-(2,5-dibromothiophen-3yl) ethanol (1.54 g, 5.4 mmol) was dissolved. To this solution, EDOT boronic ester (3.34 g, 12.5 mmol) was added, followed by the addition of PEPPSI-iPr (0.183 g, 0.27 mmol), and KF (1.87 g, 32.2 mmol). Subsequently, 15 mL of degassed water was added to the reaction mixture, which was then purged with nitrogen for 30 minutes. The mixture was subsequently heated to 85° C. for 6 hours. The progress of the reaction was followed using TLC (40% of EtOAc in pentane). After cooling to ambient temperature, the reaction mixture was filtered through a short silica pad that was subsequently washed with THE and EtOAc. The organic phase was concentrated under reduced pressure, and the residue was purified by column chromatography using a gradient of EtOAc in pentane (0->80%), yielding a yellow foam (1.05 g, 48% yield) ¹H NMR (600 MHZ, CD₃CN) δ 7.12 (s, 1H), 6.46 (s, 1H), 6.32 (s, 1H), 4.34-4.30 (m, 2H), 4.27 (ddd, J=5.5, 3.1, 1.2 Hz, 2H), 4.23 (tdd, J=3.9, 3.3, 2.1 Hz, 4H), 3.70 (td, J=6.9, 5.6 Hz, 2H), 2.85 (t, J=6.9 Hz, 2H), 2.72 (t, J=5.7 Hz, 1H). ¹³C NMR (151 MHZ, CD3CN) δ 143.2, 142.9, 139.6, 139.1, 138.4, 134.6, 127.7, 126.1, 112.1, 110.1, 100.0, 97.9, 66.1, 65.9, 65.6, 65.5, 62.6, 33.6.

Synthesis of 5,5′-(3-(2-(4-bromobutoxy)ethyl)thiophene-2,5-diyl)bis(2,3-dihydrothieno-[3,4-b][1,4]dioxine) (ETE-BuBr) (II-55). (ETE-BuBr was synthesized according to the published procedure for EDOT-BuBr⁴), A solution of ETE-OH (1.0 g, 2.45 mmol, 1 equiv.) in 20 mL DCM was added to a mixture of 1,4-dibromobutane (3.5 mL, 29.3 mmol, 12 equiv.) and tetrabutylammonium bromide (TBAB) (239 mg, 0.74 mmol, 0.3 equiv.) in 10 mL DCM. This mixture was stirred for 15 minutes before the addition of 30 ml of an aqueous 50 wt % NaOH solution. The resulting biphasic system was then stirred vigorously overnight. The progress of the reaction was followed using TLC (40% of EtOAc in pentane). The reaction mixture was diluted with 100 ml of water was added to the reaction mixture and the product was extracted using three 100 mL portions of DCM. The organic layer was dried over anhydrous Na₂SO₄, filtered, and the solvent was removed under reduced pressure. The residue was purified by column chromatography using a gradient of EtOAc in pentane (0→40%) to yield the product as a sticky yellow liquid (1 g, 75.3% yield). 1H NMR (600 MHZ, CD₃CN) δ 7.12 (s, 1H), 6.43 (s, 1H), 6.29 (s, 1H), 4.32-4.29 (m, 2H), 4.24 (td, J=3.6, 2.0 Hz, 2H), 4.21 (ddt, J=6.3, 4.0, 2.3 Hz, 4H), 3.58 (t, J=6.7 Hz, 2H), 3.44 (t, J=6.8 Hz, 2H), 3.40 (t, J=6.2 Hz, 2H), 2.87 (t, J=6.7 Hz, 2H), 1.90-1.81 (m, 2H), 1.65-1.58 (m, 2H). ¹³C NMR (151 MHZ, CD₃CN) δ 143.15, 142.86, 139.57, 139.01, 138.34, 134.56, 127.61, 126.15, 112.14, 110.06, 100.01, 97.92, 70.96, 70.36, 66.07, 65.84, 65.55, 65.44, 35.27, 30.67, 30.52, 29.03.

Synthesis of 3-(4-(2-(2,5-bis(2,3-dihydrothieno[3,4-b][1,4]dioxin-5-yl)thiophen-3-yl)-ethoxy)butyl)-1,2-oxathiane 2,2-dioxide (ETE-BuSultone) (II-53). To a solution of 1,4-butane sultone (170 μL, 1.66 mmol) in 6 mL dry THF at −78° C. under an inert nitrogen atmosphere, n-BuLi (2.5 M in hexanes, 1.85 mmol, 740 L) was added dropwise. The mixture was stirred at −78° C. for 30 minutes. Subsequently, a solution of ETE-BuBr (897 mg, 1.65 mmol) in 6 ml of dry THF was added, resulting in the immediate formation of a pale red solution. The reaction mixture was further stirred at −78° C. for 30 minutes, after which the cooling bath was removed to allow the reaction to proceed at ambient temperature overnight. The reaction was quenched using a small amount of water, and the solvents were removed under reduced pressure. The residue was redissolved in 100 ml of water and extracted with two 100 ml portions of EtOAc. The organic phase was dried over anhydrous Na₂SO₄ and filtered, and the solvent was removed under reduced pressure. The crude was purified by column chromatography using a gradient of EtOAc in pentane (0->100%). The title compound was obtained as a yellow foam (393 mg, 40% yield). ¹H NMR (600 MHZ, CD₃CN) δ 7.13 (s, 1H), 6.46 (s, 1H), 6.32 (s, 1H), 4.42 (ddd, J=8.9, 3.4, 1.7 Hz, 2H), 4.34-4.31 (m, 2H), 4.26 (ddd, J=5.4, 3.1, 1.1 Hz, 2H), 4.25-4.20 (m, 4H), 3.60 (t, J=6.7 Hz, 2H), 3.40 (t, J=6.0 Hz, 2H), 3.06 (dddd, J=11.1, 7.7, 5.7, 3.8 Hz, 1H), 2.89 (t, J=6.7 Hz, 2H), 1.92-1.71 (m, 4H), 1.57-1.44 (m, 4H), 1.44-1.36 (m, 1H).

Synthesis of 8-(2-(2,5-bis(2,3-dihydrothieno[3,4-b][1,4]dioxin-5-yl)thiophen-3-yl)ethoxy)-1-(trimethylammonio)octane-4-sulfonate (ETE-BuSA) (II-4). Trimethylamine (1.3 mL, 5.46 mmol, 14.8 equiv.) as a 4.2 M solution in ethanol, was added to a degassed solution of ETE-BuBr (0.220 g, 0.37 mmol, 1 equiv.) in 1.5 mL of dry acetonitrile in a 15 mL pressure tube. The tube was heated to 85° C. overnight. After cooling to ambient temperature, the crude solution was transferred to a small flask, and the solvents were removed under reduced pressure, yielding the title compound as a pale-yellow foam in a sticky yellow liquid. The residue was subjected to column chromatography (EtOAc:MeCN:MeOH:H₂O 3:1:1:1) in a fluffy yellow solid (145 mg, 60% yield). ¹H NMR (800 MHZ, CD₃OD) δ 7.15 (s, 1H), 6.46 (s, 1H), 6.32 (s, 1H), 4.37-4.32 (m, 2H), 4.30-4.26 (m, 2H), 4.26-4.21 (m, 4H), 3.65 (td, J=6.7, 3.1 Hz, 2H), 3.48 (t, J=6.0 Hz, 2H), 3.28 (dt, J=12.2, 6.0 Hz, 1H), 3.20 (dd, J=12.1, 5.4 Hz, 1H), 3.07 (s, 9H), 2.91 (t, J=6.7 Hz, 2H), 2.68 (tt, J=8.1, 4.2 Hz, 1H), 1.98 (tdd, J=15.4, 8.3, 4.4 Hz, 3H), 1.75 (dddd, J=14.7, 9.4, 7.4, 5.5 Hz, 1H), 1.69-1.60 (m, 2H), 1.60-1.52 (m, 3H), 1.49-1.43 (m, 1H). ¹³C NMR (201 MHz, CD₃OD) δ 143.59, 143.26, 139.77, 139.20, 138.37, 135.22, 128.01, 126.24, 112.80, 110.62, 100.15, 97.84, 71.49, 67.77, 66.42, 66.17, 65.89, 65.76, 60.21, 53.56, 31.16, 30.78, 30.74, 27.60, 24.84, 21.41. HRMS (ESI) m/z: [M+H]⁺ calcd for C₂₉H₄₀NO₈S₄: 658.1637, found: 658.1649.

### Ex-Vivo Studies

The proBICS comprising a mixture of A5 and ETE-BuSA was injected into agarose to form a dark structure in and around the injection track. Contacting the proBICS using external electrodes and supplying a low voltage allowed the ETE-BuSA to attach to the A5 and form a stable gel electrode inside the agarose. The formation of BICS can be observed from a darkening of the structure and higher magnification revealed dendritic structures extending into the agarose similar to how other trimers react.

Electropolymerization significantly increased the specific capacitance of BICS, reaching values between 30 and 40 F/cm³, in vitro. This improvement is important since it relates directly to the device's capacity to retain electrical charge, thereby enhancing its overall performance and efficiency according to the inventive concept. The decrease in impedance resulting from electropolymerization is another parallel advantageous result which signifies easier electron/ion movement in the BICS structure, essential for fast and effective charge/discharge processes while behaving as a transistor. The decrease in impedance is linked to the documented expansion of the BICS volume.

After establishing that the injectable proBICS solution can be stabilized in situ through electropolymerization, with excellent electrical properties, the mechanical properties were explored. Using a mechanical indenter, the static and dynamic properties of the BICS was investigated in an agarose gel by either gently pushing in/out or by applying a sinusoidal indenter displacement. It is noteworthy that the mechanical compatibility of BICS with cardiac tissue has been demonstrated quantitatively by measuring parameters such as Young's modulus, stress relaxation, and cyclic strain testing. BICS exhibited values that were closely aligning with those of native heart tissue. The results emphasize the capability of BICS for seamless integration and operation in cardiac applications.

By isolating zebrafish hearts ex vivo offers a controlled arena for understanding the relationship between BICS and cardiac tissue. The hearts can be kept beating for more than one hour post excision. Applying proBICS and then electropolymerizing showed no observable damage to the heart and a maintained beating frequency further corroborating the biocompatibility of the material.

The BICS on the heart was contacted (counter electrode in surrounding buffer) and used to relay external voltage pulses resulting in an increase of the beating frequency. Initially, the ex vivo heart was beating at a native frequency of 0.8 Hz. Upon applying a 2 Hz external electrical stimulation, the heart's beating rate adjusted to match this frequency, demonstrating its responsiveness to external electrical stimuli from the BICS touching it. After ceasing the stimulation, the heart naturally returned to its original beating frequency of 0.8 Hz.

### In Vivo Studies

Anesthetized fish were microinjected with proBICS using metal coated capillaries. Upon retraction of the injection capillary, additional proBICS was injected to form a continuous structure extending from the pericardium up to the surface where an additional proBICS patch was deposited. The metal coated microinjection capillary was placed on the patch and connected to an external voltage supply to drive the electrofunctionalization (counter electrode placed under the fish). Post functionalization, BICS forms around the heart and the fish were either woken up, kept under anesthesia, or euthanized depending on which assay was to be used.

In vivo electrochemical analysis provided insights consistent with the in vitro findings. Specifically, the electrofunctionalization of proBICS within the pericardium resulted in a notable reduction in impedance, indicative of a shift towards lower frequencies. This alteration suggests an increase in the capacitance of the BICS relative to its state as pro-BICS before electrofunctionalization and such a change highlights an overall improvement in electrochemical behavior of the material.

One of the most common, and least invasive ways to monitor heart activity is through the use of electrocardiograms (ECGs). Due to their small size, ECGs recorded from zebrafish is typically obtained using three electrodes (compared to 12 electrodes for most human ECGs).

Recording of ECGs from zebrafish were initiated. This allowed for comprehensive analysis and comparison of the beating profiles of hearts with BICS against those of a control group. Before starting to record ECGs from zebrafish, it is essential to immobilize them using a paralyzing agent, such as tricaine, which is a sodium channel blocker. Tricaine, the only FDA-approved anesthetic for this purpose, is widely adopted by the research community for zebrafish anesthesia, despite its potential to affect zebrafish heart rate. In the present study, the depth of anesthesia was carefully controlled and the procedure duration time to minimize potential effects from tricaine. On the other hand, for ensuring stable and reproducible results with ECG sampling, it necessitates the implementation of appropriate high-pass and low-pass filters, particularly with a 1 kHz sampling frequency. By using a 0.3 Hz high-pass filter, a 1 kHz low-pass filter, and a 50 Hz notch filter, raw ECG signals were successfully obtained and characterized by an isoelectric baseline within a 2 mV range, effectively minimizing the impact of noise. Recording an electrocardiogram (ECG) from a zebrafish poses challenges due to the small size of the fish, and electrode placement can result in a low signal-to-noise ratio. To address this, a literature procedure was employed. The noteworthy similarity in R and T wave concordance between zebrafish and human normal ECGs enhances the clinical relevance of the zebrafish heart model as a surrogate for comprehending human cardiac electrophysiology. Similar to human ECG recordings, which are susceptible to noise from various sources like power line artifacts, electrode contact noise, and muscle movement artifacts, previous studies have outlined difficulties in adult zebrafish ECG signals.⁸ These challenges encompass considerable variability in waveform morphology and variations in QT and QTc intervals due to interference. The variation within the morphology and amplitude of the signal among different fish can be attributed to inherent differences in their cardiac physiology, body shape, and the variability of electrode placement, despite efforts to consistently place electrodes in the same locations each time. The raw ECG signal of the zebrafish closely mirrors that of a human ECG, showcasing distinct peaks such as the P wave, QRS complex, and T wave, identifiable without the need for signal processing. On the other hand, given BICS is a conductive structure, its widespread coverage over the heart can result in situations where the P waves are obscured by interference. BICS' conductive characteristics can cause electrical signals that disrupt the detection of P waves, making them hard to differentiate from background noise. To gain deeper insight, ECG recordings were also conducted from the BICS patch applied to the skin of the fish. The results corroborated the previous observations, yet the P waves remained distinguishable. Consequently, in order to identify impacts on the ECG spectra, focus was put on analyzing the time difference between two consecutive R peaks. This approach allowed precise evaluation of the BICS patch's effect on cardiac rhythm, providing a more nuanced understanding of its impact on heart function.

When stimulating the heart using the injected BICS, the low voltage electrical signals from the heart (0.1 mV) was masked by the comparably high stimulation voltage (4 V). The overlap in frequency made it impossible to filter out the stimulation pulses. Due to this reason, the attention was turned to a mechanical method for recording the heart beats, seismocardiography.

A small spherical indenter was utilized to detect the stress and relaxation at the apex of the thoracic cavity, which enabled tracking of the cardiac movement while still having the stimulating electrodes on the BICS patch. Seismography results were correlating with ECG results by showing the QRST complex. The results of the comparative analysis of main electrocardiograms demonstrated similarities in the dynamics of electrocardiogram and seismocardiogram signals while seismocardiography gave the advantage of recording the heartbeat without electrodes and wires. When in vivo stimulation periods were recorded with seismography, the heartbeat signal could be seen and it was clearly following the stimulation peaks at 2 Hz. When the obtained results Fourier transferred, it was concluded that the native heartbeat around 60 bpm had increased to 120 bpm (2 Hz) and the QRST complex was prominent. Interestingly, some fish experienced irregular heart rhythm, arrythmias, which could be recovered using the BICS stimulator. Uneven rhythms, extra beats, and missing beats in the spectra were observed. During BICS stimulation, the arrhythmic heart beats synchronized and showed no signs of the arrythmia. After the stimulation cycle, the hearts went arrythmic again. Control experiments conducted on fish without BICS highlighted the necessity for hearts to be stimulated externally by BICS, thus the external pulses had no effect on heart beating.

BICS Concept and Design: The BICS, envisioned as a compact, bioresorbable, and injectable device, serves as an alternative to traditional, surgically implanted defibrillators and pacemakers. Designed for short-term use, especially in remote or difficult-to-access areas, BICS offers a less invasive solution for temporary cardiac stimulation until permanent treatment can be arranged.

Injection and Placement Methodology: BICS can be accurately placed using imaging guidance like ultrasound or anatomical landmarks in emergency situations, minimizing the need for open surgery and allowing for its use in challenging conditions such as war zones or remote regions.

Material and Formulation Challenges: Creating injectable electrodes for cardiac applications that adhere to the heart's surface without disrupting its movements presents significant challenges. The pro-electrode formulation, a mixture of A5 and ETE-BuSA, addresses these by being highly soluble for injection, forming conductive structures in vivo that match the cardiac tissue's elasticity and stiffness. Another mixture, EEE-COOH, ETE-S, and A5, shows the combination of two trimers and A5, both electrofunctionalization and enzymatic polymerization.

Zebrafish and Chicken Embryo Models: Zebrafish and chicken embryos are utilized for in vivo studies due to their anatomical and physiological similarities to human cardiac systems, providing a viable model for assessing BICS's efficacy and biocompatibility.

Ex-vivo and In-vivo Studies: Studies demonstrated the ability of BICS to stimulate cardiac activity effectively, with injected proBICS forming a stable gel electrode and showing no observable damage to cardiac tissue. In vivo studies further validated BICS's efficacy in stimulating heartbeats and its biocompatibility, with animals showing no behavioral changes post-bioresorption.

Mechanical and Electrochemical Analysis: Mechanical compatibility with cardiac tissue was demonstrated through parameters such as Young's modulus and stress relaxation. Electrochemical analysis in vivo showed reduced impedance and increased capacitance post-electropolymerization, indicating improved material behavior.

ECG and Seismocardiography Studies: ECG recordings from zebrafish provided insights into the cardiac activity influenced by BICS, with adjustments in the beating frequency observed upon electrical stimulation. Seismocardiography offered a mechanical method to record heartbeats, corroborating ECG findings and highlighting BICS's ability to correct arrhythmic heartbeats.

### Example 4: Photopolymerization In Vivo Formation of Conductive Bioelectronics

### Background

The present inventive concept relates to spatial control of the formed conductive polymers or polymer electrodes. In particular, the present inventive concept relates to specific targeting of tissue structures and improved 3D control. Polymerization with spatial control has been achieved using photopolymerization with and without photo masking. The present disclosure offers a composition of the first aspect with optimized oxidation potential which enable photocatalyzed formation of conductive bioelectronics in live zebrafish with spatial control.

### A5-EEE-S Solution

20 μl surfactant solution was added to 0.8 mg EEE-S. To this solution, 1 μL 10 mM rose Bengal in MQW was added. The solution was oxygenated by bubbling oxygen through the solution. The oxygenated EEE-S solution was added to a vial containing 0.2 mg A5. The solution was sonicated for one minute. The final concentration of the solution corresponds to 40 mg/ml EEE-S, 10 mg/ml A5 and 0.4 mM rose Bengal. The solution was freshly prepared for each experiment and immediately consumed after preparation.

### Photopolymerization in Microtiter Plates

A solution of trimer (2 μL, 20 mg/ml in MilliQ water), optional photocatalyst (1 μL, 10 mM in MilliQ water for RoseBengal and DMSO for SIR—COOH) and MilliQ water were added to a black clear bottom 96-well microtiter plate to get a total volume of 100 μL. The solution was illuminated by light (UV 385 nm, Green 550 nm, or Red 621 nm, D-LEDI Nikon) for a specified time. The absorbance spectrum (280-1000 nm) was then recorded (Tecan SparkCyto 400).

### Catalyst Loading

Performed according to the general method photopolymerization in microtiter plates using EEE-COONa (III-5) (20 mg/ml in MilliQ water) at 1, 4, 13, 40 and 113 mol % of RoseBengal (10 mM in DMSO).

### Photopolymerization Using Agarose Mold

### Wavelength Specific Photopolymerization

A trimer solution (2 μL, formulated using 15 μL EEE-COONa (20 mg mL⁻¹ in MilliQ water) with and without 1 μL Rose Bengal (10 mM in DMSO)) was injected as two parallel lines into an agarose mold (0.5% agarose in Ringer solution) using a Hamilton syringe. The agarose mold was transferred to the microscope setup (Nikon ECLIPSE FN1) which was then illuminated by green light for 5 min followed by UV light (385 nm) for 5 min using a 4×/0.10 Nikon objective. Imaging was performed with the same objective.

The conductivity of the A5 and A5-ETE-S in agarose was measured using a two-terminal setup in which 25 μm Au-coated tungsten microprobes (Signatone, Gilroy, CA) were connected to the polymer embedded in the agarose. By sweeping an applied electric potential and registering the resulting current over different distances, conductivity can be estimated using the transmission line model.

### Spatial Controlled Photopolymerization

EEE-COONa (5 μL, 20 mg mL⁻¹ in MilliQ water) was added to the surface of an agarose mold (0.5% agarose in Ringer solution) and the solution was dried in forming a thin layer of the trimer. Two 3D printed photolithography masks were inserted into the light path (ND filter slots). The agarose mold was transferred to the microscope setup (Nikon ECLIPSE FN1) which was then illuminated by UV light (385 nm) for 5 min with Mask 1 followed by Mask 2 for 5 min using a 20×/0.45 Nikon objective. Imaging was performed using a 4×/0.10 Nikon objective.

### Pattern and Electric Measurements

A5-EEE-S solution (2 μL, see preparation of A5-EEE-S solution) was added to the surface of a glass slide. A 1×2×0.5 cm agarose mold was placed on top of the trimer solution (0.5% agarose in Ringer solution). A 3D printed photolithography mask was inserted into the light path (ND filter slots). The agarose mold was transferred to the microscope setup (Nikon ECLIPSE FN1) which was then illuminated by Green light (561 nm) for 15 min using a 20×/0.45 Nikon objective. Imaging was performed using a 4×/0.10 and a 20×/0.45 Nikon objective. The patterned agarose was removed from the glass slide and washed with 3 ml MilliQ water.

The agarose mold was then placed on interdigitated Au electrodes connected to a Keithley sourcemeter 2612B (Keithley Instruments). The pattern was facing towards the Au electrodes. Two of the interdigitated electrodes were contacted using external microelectrodes. An applied voltage was swept, and the resulting current was registered. This was repeated for all interdigitated electrode leads covering the conductive polymer. The distance between adjacent electrodes was 15 μm, and the width was 2.5 mm.

### Photopolymerization Ex Vivo

The trimer solution contained 15 μL EEE-COONa (20 mg mL⁻¹ in MilliQ water) and 1 μL Rose Bengal (10 mM in DMSO). Before microinjection and photo polymerization, larva (Casper mutant (Tg(elav3:GCaMP6f)) on nacre background) were euthanized with tricaine (ethyl 3-aminobenzoate methanesulfonate; 0.2 mg mL⁻¹) until movements had ceased and the fish did not respond to vibrations caused by tapping close to the tricaine container minimum for 10 min. The larva was placed on its side on a plate filled with 1% agarose (Agarose, LE, Analytical Grade, Promega Corporation) in E3 medium that had been allowed to solidify. The plate was then transferred, to the microinjection setup, and a capillary filled with the trimer solution with a 30 μm diameter bevelled tip (cat. No. BM100T-15. Bevelled, straight, shortened and firepolished ends from Biomedical-Instruments GMBH) was inserted into the ventricle. The total injection volume was estimated to be 1 nL. After injection, the larva was transferred to the microscope setup (Nikon ECLIPSE FN1) and the head was illuminated by green light for 15 min using a 4×/0.10 Nikon objective. Photopolymerization was confirmed by imaging using brightfield light and UV (385 nm) with the same objective.

Ex Vivo Patterning Procedures were Performed on Excised Brain-Dura Complexes

For patterning, 2 μL A5-EEE-S solution (prepared as described above) was injected into the subdural space on top of the interhemispheric space of Brain-dura complexes using a 10 μL Microliter Syringe (Hamilton Company). Injected samples were placed on a plate filled with 1% agarose (Agarose, LE, Analytical Grade, Promega Corporation) in Ringer medium that had been allowed to solidify with the injection site orientated ventrally. The plate was then transferred to the photopolymerization setup, and a lithography mask was inserted into the lightpath/ND-filter slot of the microscope. The pattern was focused on the surface of the sample and irradiated with 541 nm light for 15 min. Imaging was performed using a 4×/0.10 Nikon objective.

### Photopolymerization In Vivo—Tail Fin Patterning/Lithography

Adult zebrafish (Danio rerio) AB wildtype were used for the patterning experiment. Before the patterning procedure, fish were anesthetized with tricaine medium (final concentration 0.2 mg/ml) until opercular movements had ceased and the fish did not respond to vibrations caused by tapping close to the tricaine container. The anesthetized fish was placed on its side on a plate filled with 1% agarose (Agarose, LE, Analytical Grade, Promega Corporation) in Ringer medium that had been allowed to solidify. A piece of moist tissue paper was placed over the fish to keep the body from drying but still exposing the caudal fin. The caudal peduncle was carefully lifted with tweezers so that a glass plate [data of glass slide] could be slid under the caudal fin. The caudal fin was desiccated by blotting it with a paper towel. 3 μL freshly prepared EEE-S:A5 mix were applied by gently lifting the fish at the caudal peduncle with a pipette containing the mix. The pipette tip was pivoted towards the tail fin, and the mix was extruded between tail fin and glass slide. The plate was then transferred to the photopolymerization setup, and a lithography mask was inserted into the lightpath/ND-filter slot of the microscope. The pattern was focused on the surface of the sample and irradiated with 550 nm light for 15 min. After patterning, excess material was washed off the tail fin. Fish were revived directly by flushing the gills with fresh aquarium water and transferred to a post-op aquarium for observation.

### Results

It was an object to identify compounds of formula (I), (II) and/or (III) that forms conductive organic polymers after photopolymerization but also that have optimal water solubility and biocompability for in vivo applications. By using a microtiter plate based method to efficiently evaluate compounds, i.e. trimers, and conditions, a fluorescence microscope with a LED light sources with wavelength covering the visible light region from far UV to red was used. The far UV wavelength (385 nm) aligns well to the absorbance peak of e.g. the ETE and EEE trimers between 350 and 400 nm for efficient excitation. Conversion of the trimers and formation of the product was monitored by spectrophotometry analysis (absorbance scan). However, ETE trimers did not form the same type of product (e.g. spectrum) when exposed to light compared to after enzymatic conditions with HRP and H₂O₂. There was quite a large absorbance peak at 350-400 nm similar to the trimers which could indicate ineffective conversion. But this peak was not reduced with longer reaction times pointing towards a product still with three conjugated thiophene units but with unknown structural modifications.

Trimers EEE-S, EEE-COONa and EEE-PC were synthesized and redox analysis showed that indeed the redox potential was lower for the EEE scaffold compared to the ETE. Further, indicative of the higher reactivity already exposure to natural light in the lab darkened both the dry powder and the aqueous solutions of the EEE trimers and it is thus necessary to store the EEE trimers at −80° C. When exposed to far UV light (385 nm), these trimers formed a dark blue-green solution within 5 minutes typical for PEDOTs with overlapping spectrum to the enzymatic process with a broad peak around 600 nm. This was a good starting point for further evaluations but even far UV light is not compatible with biological systems, damaging to the tissue and also faces problems with limited penetration due to light scattering and absorption. Ideal would be to be able to use longer wavelengths green or red light to reduce these effects. Photo catalysts have for example been used to enable longer wavelength photopolymerization in tissue.

Further, catalytic amounts of Rose Bengal was used to photopolymerize EEE-COONa with green light and achieves similar results as without photocatalyst with far UV within 5 minutes. This was encouraging for in vivo experiments with zebrafish, as reaction time is crucial. The reaction was also efficient with 1% catalyst loading, the reaction almost proceeded to completion within 5 minutes and with 4% and above 100% conversion was achieved.

Furthermore, it was also an object to identify photocatalysts which could be used with red light for further improvement of in vivo applications. SIR—COOH is a relatively new analog of the commonly used class of fluorescent dyes rhodamine were the bridging oxygen of rhodamines is replaced with a dimethyl silyl group. Interestingly, the replacement of O to Si (Me) 2 red shift the excitation and emission spectrum with 70-100 nm still maintaining the original brightness. Thus, the excitation maximum is 650 nm for SIR—COOH in the lower range of the first NIR window in tissues (approximately 650-1000 nm). SIR—COOH has recently been used in a bioortogonal photoclick reactions for cell based applications further supporting its suitability for in vivo applications but application in oxidative photopolymerizations have not been evaluated before. SIR—COOH as photocatalyst at 621 nm red light functioned similar to Rose Bengal and lead to the conversion of the EEE-COONa trimer. However, the reaction efficiency was lower compared to reactions with Rose Bengal and there was some remaining trimer after 5 min. The absorbance spectrum for enzymatic and photo reaction fairly well overlapped for EEE trimers but with conditions using SIR—COOH as catalyst there was an additional peak spectrum at 800 nm which was not seen when the photo reaction was performed with Rose Bengal or without catalyst using far UV. Further analysis using Maldi-MS analysis of reaction mixtures from SIR—COOH and far UV conditions showed the formation of dimers in both cases but trimers only when using SIR—COOH as photocatalyst. This could be seen both for EEE-S and EEE-COONa. IT was speculated that the peak at 600 nm is the hexamers and the peak at 800 nm the nonamers. In such case the red light at 621 nm in the SIR—COOH catalyzed reactions could excite the dimer and SIR—COOH activate the monomer thus generate a monomer-dimer reaction which could form nonamers. Addition of more trimer to already completed reactions followed by further red light exposure increased the peak at 800 nm compared to the peak at 600 nm which could support a dual activation mechanism in the SIR—COOH catalyzed reaction. When the trimer concentration was increased from 0.4 mg/ml to 4 mg/ml the reaction efficiacy declined and most of the trimer was remaining after 5 min. The trimer in itself absorb the light, thus the conversion is dependent on the length of the light path in the trimer solution (i.e. cross section) and this could be nicely illustrated by reducing the volume in the well and thereby get complete conversion after 5 minutes. These initial studies in solution showed that EEE-PC is least stable in aqueous solution compared to EEE-S and EEE-COONa, which leads to that these trimers may be preferred in further photopolymerization studies.

### Itemized List of Embodiments

Item 1. A composition comprising a co-polymer of formula (I)

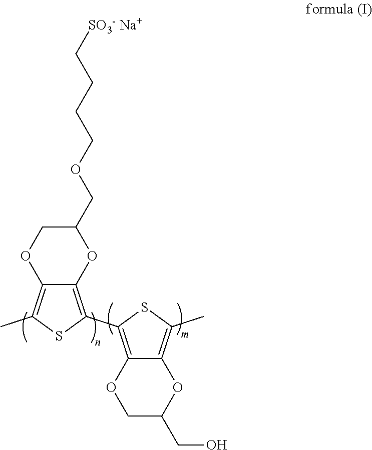

wherein n is 2-25, and m is 0-1n; and
a compound of formula (II) and/or a compound of formula (III):

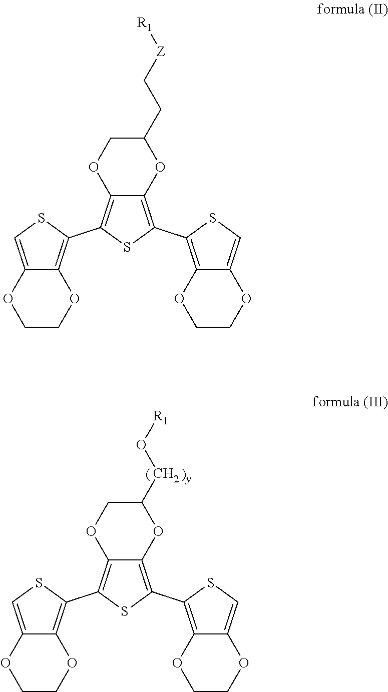

wherein Z is selected from —O— or —CH₂—; each y is an integer selected from the group consisting of 0, 1, 2, 3 and 4, and
each R¹ is selected from the group consisting of optionally substituted phosphate, optionally substituted phosphate esters and derivatives thereof, optionally substituted amine, and optionally substituted amide.

Item 2. The composition of item 1, further comprising an aqueous solvent.

Item 3. The composition of item 2, wherein the aqueous solvent is water.

Item 4. The composition of any of items 1-3, wherein the compound of formula (III) is absent.

Item 5. The composition of any of items 1-3, wherein the compound of formula (II) is absent.

Item 6. The composition of any of items 1-5, wherein R¹ is selected from the group consisting of optionally substituted phosphatidylcholine, optionally substituted phosphatidylamine, optionally substituted C₁₋₁₀ alkyl sulfonate, optionally substituted and alkoxylated sulfonate, optionally substituted C₁₋₁₀ alkylamine, optionally substituted and alkoxylated amine, optionally substituted C₁₋₁₀ alkyl carboxylate, and optionally substituted and alkoxylated carboxylate.

Item 7. The composition of item 6, wherein R¹ is alkoxylated it is ethoxylated.

Item 8. The composition of any of items 1-5, wherein R¹ is phosphatidylcholine.

Item 9. The composition of any of items 1-5, wherein R¹ is phosphatidylcholine substituted by an ethoxylated azide.

Item 10. The composition of any of items 1-4, wherein the compound of formula (II) is selected from the group consisting of

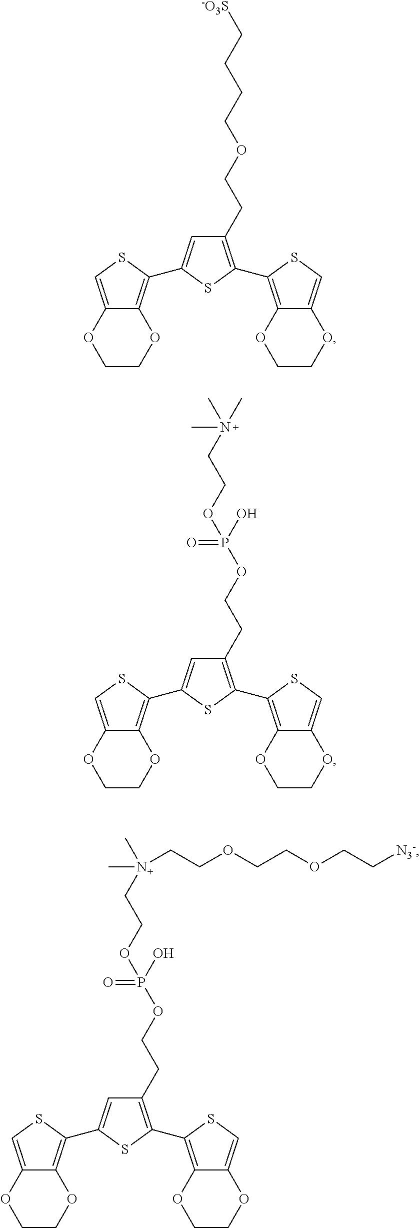

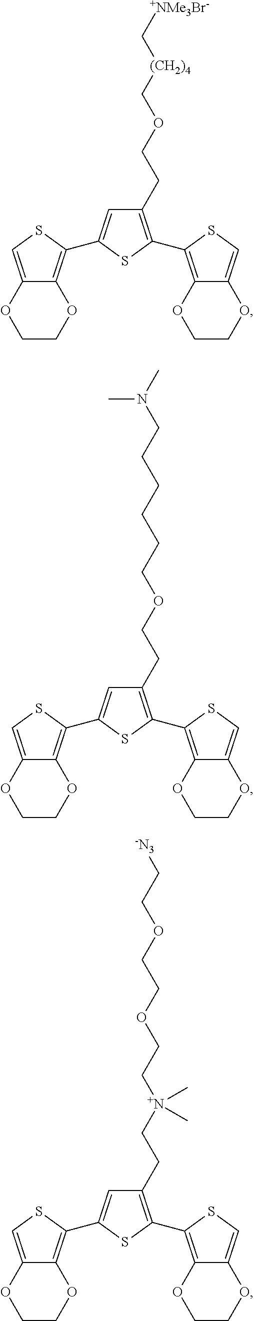

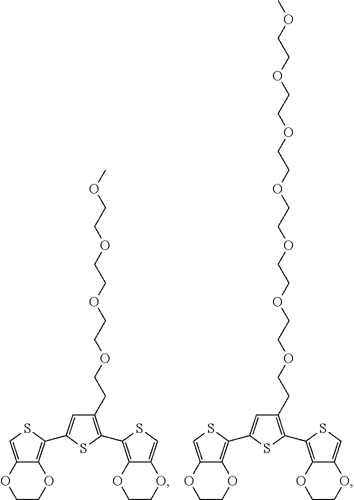

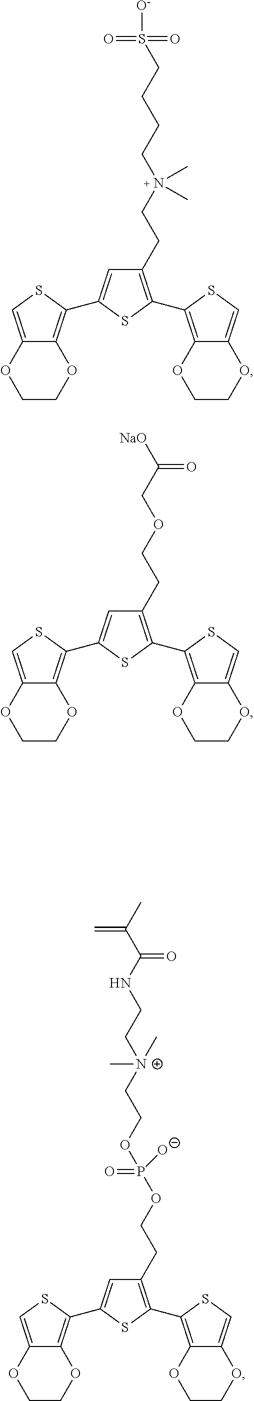

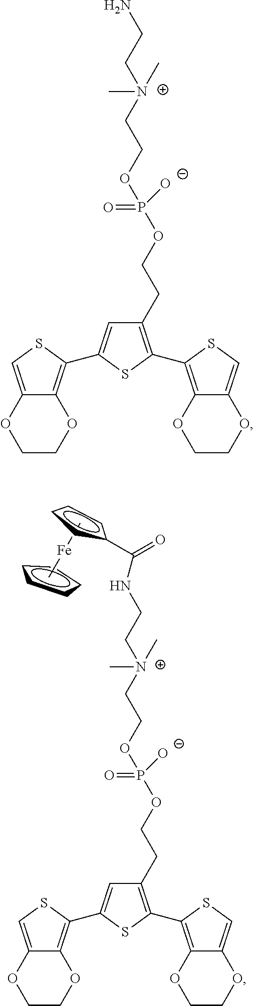

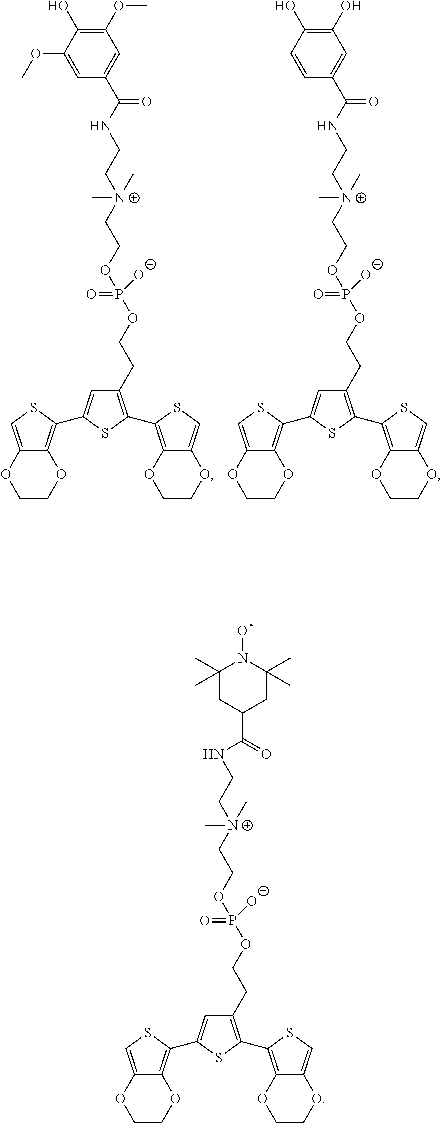

Item 11. The composition of any of items 1-3 and 5, wherein the compound of formula (III) is selected from the group consisting of

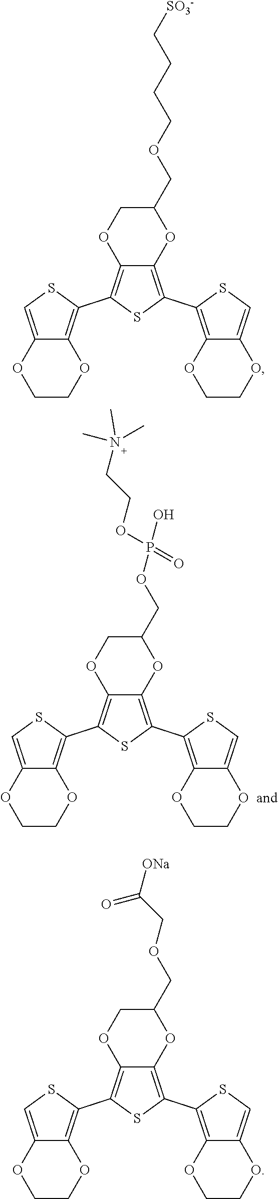

1. A composition comprising a polymer of formula (I) and one or more compound of formula (II) or formula (III),
wherein
the polymer of formula (I) is represented by the structure: 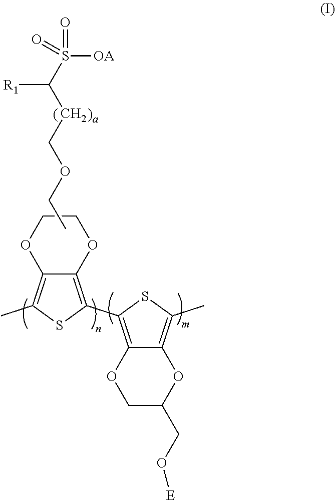
the one or more compound of formula (II) or formula (III) is represented by the structures: 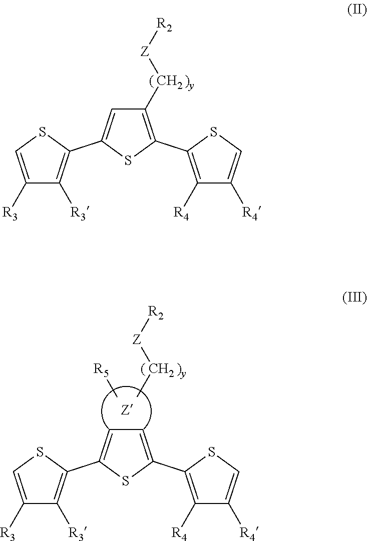
and wherein
each A is independently H, Na, K, Li, Ca, Mg, Sr, or Ba;
E is H, C₁₋₆ alkyl, C₂₋₆ alkenyl, C₂₋₆ alkynyl, C₃₋₆ cycloalkyl, aryl, heteroaryl, or —(CH₂CH₂O)_{q}CH₂CH₂OH, wherein said C₁₋₆ alkyl is optionally substituted with one or more of —N₃, —OH, —SO₃A, —N(C₁₋₆ alkyl)₂, —NH⁺(C₁₋₆ alkyl)₂, or —N⁺(C₁₋₆ alkyl)₃;
R₁ is H, C₁₋₆ alkyl, C₂₋₆ alkenyl, C₂₋₆ alkynyl, C₃₋₆ cycloalkyl, aryl, or heteroaryl, wherein said C₁₋₆ alkyl is optionally substituted with one or more-N(C₁₋₆ alkyl)₂, —NH⁺(C₁₋₆ alkyl)₂, or —N⁺(C₁₋₆ alkyl)₃;
each Z is individually a bond, —O—, —OP(O)(0-)O—, —OP(O)(OH)O—, —OC(O)—, —C(O)O—, or —OC(O)NH—;
each R₂ is H, C₁₋₂₀ alkyl, aryl, heterocyclyl, heteroaryl, or Si(C₁₋₆ alkyl)₃, wherein said C₁₋₂₀ alkyl, aryl, heterocyclyl and heteroaryl is optionally substituted with one or more R₅ or R₆;
each of R₃, R₃′, R₄ and R₄′ is H or —(CH₂)_{y}—Z—(R₂),
when R₃ and R₃′ are C₁₋₆ alkoxy and taken together with the atom to which they are each attached form a heterocycle optionally substituted with one or more R₅ or R₂;
when R₄ and R₄′ are C₁₋₆ alkoxy and taken together with the atom to which they are each attached form a heterocycle optionally substituted with one or more R₅ or R₂;
R₅ is selected from H, C₁₋₆ alkyl, C₂₋₆ alkenyl, and C₂₋₆ alkynyl;
Z′ is 6- or 7-membered heterocycle;
each R₆ is —SO₃A, —CO₂A, —CO₂(R₉), —OH, —O(R₉), halogen, —N₃, —NH₂, —NH(R₉), —NHC(O)(R₉), —N(C₁₋₆ alkyl)₂, —N⁺(C₁₋₆ alkyl)₃, —(OCH₂CH₂)_{q}—(R₈), C₁₋₆ alkyl, C₂₋₆ alkenyl, C₂₋₆ alkynyl, aryl, heteroaryl, heterocyclyl, ferrocenyl, or —C(O)NH(C₁₋₆ alkyl), wherein said-N⁺(C₁₋₆ alkyl)₃ and —C(O)NH(C₁₋₆ alkyl) are each optionally substituted with one or more R₇, and said C₁₋₆ alkyl, heterocyclyl, heteroaryl, and aryl are each independently substituted with one or more R₇, R₉ or R₁₀;
R₇ is —SO₃A, —CO₂A, —(OCH₂CH₂)_{q}—(R₈), —NH₂, —NHC(O)—(R₉), —OC(O)—(R₉), aryl, guanidinyl, or —C(O)NH(C₁₋₆ alkyl), wherein said guanidinyl and —C(O)NH(C₁₋₆ alkyl) are each independently optionally substituted with one or more R₈;
R₈ is C₁₋₆ alkyl, C₁₋₆ alkoxy, —OH, —NH₂, —NH(C₁₋₆ alkyl), —N₃, —OC(O)—(R₉), or
—C(O)NH(C₁₋₆ alkyl), wherein said C₁₋₆ alkyl and —C(O)NH(C₁₋₆ alkyl) are optionally substituted with one or more R₉;
R₉ is C₁₋₆ alkyl, C₂₋₆ alkenyl, C₂₋₆ alkynyl, aryl, heteroaryl, heterocyclyl, ferrocenyl, —B(OH)₂, —CO₂A, or —CO₂(C₁₋₆ alkyl), wherein said C₁₋₆ alkyl, aryl, heterocyclyl and —CO₂(C₁₋₆ alkyl) are each independently optionally substituted with one or more of R₁₀;
R₁₀ is -oxo, —SO₃A, —NH₂, —CO₂A, —OH, —P(O)(OH)₂, C₁₋₆ alkyl, C₁₋₆ alkoxy, aryl, or heteroaryl;
n is 4-32;
m is 0-10;
a is 1-5;
y is 0-16; and
q is 0-15;
or a pharmaceutically acceptable salt thereof.

2. The composition of claim 1, wherein
m is 0 or 1,
E is H, C₁₋₆ alkyl, —(CH₂CH₂O),CH₂CH₂OH, C₁₋₆ alkyl substituted with —N₃, C₁₋₆ alkyl substituted with-SO₃A, or C₁₋₆ alkyl substituted with-SO₃A and —N⁺(C₁₋₆ alkyl)₃, and
R₁ is H, C₁₋₆ alkyl, or C₁₋₆ alkyl substituted with —N⁺(C₁₋₆ alkyl)₃.

3. The composition of claim 1, wherein m is 1, E is H, and R₁ is H or C₁₋₆ alkyl.

4. The composition of claim 1, wherein m is 0 and R₁ H or C₁₋₆ alkyl substituted with —N⁺(C₁₋₆ alkyl)₃.

5. The composition of claim 1, wherein said composition comprises the polymer of formula (I) and one or more compounds of formula (II-a), formula (III-a), formula (III-b), formula (III-c), and formula (III-d): 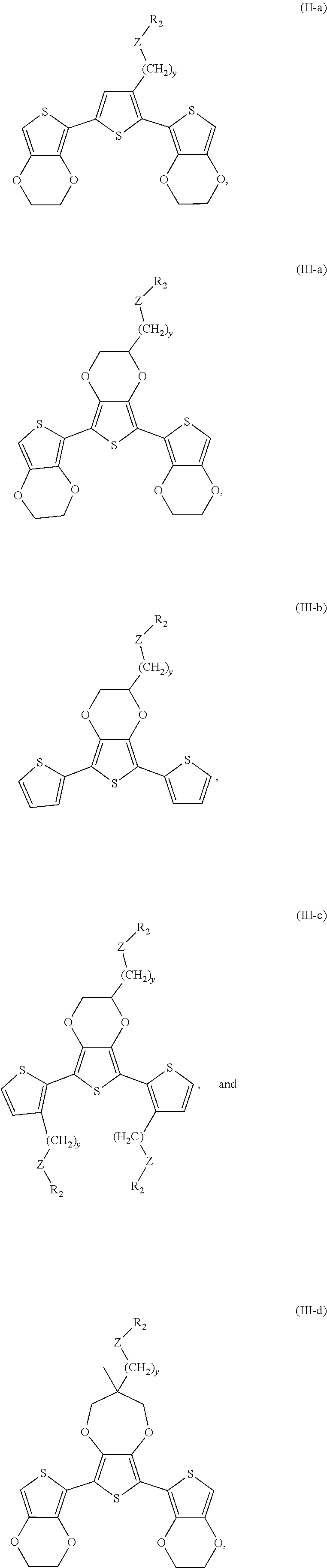
or a pharmaceutically acceptable salt thereof.

6. The composition of claim 1, wherein R₂ is 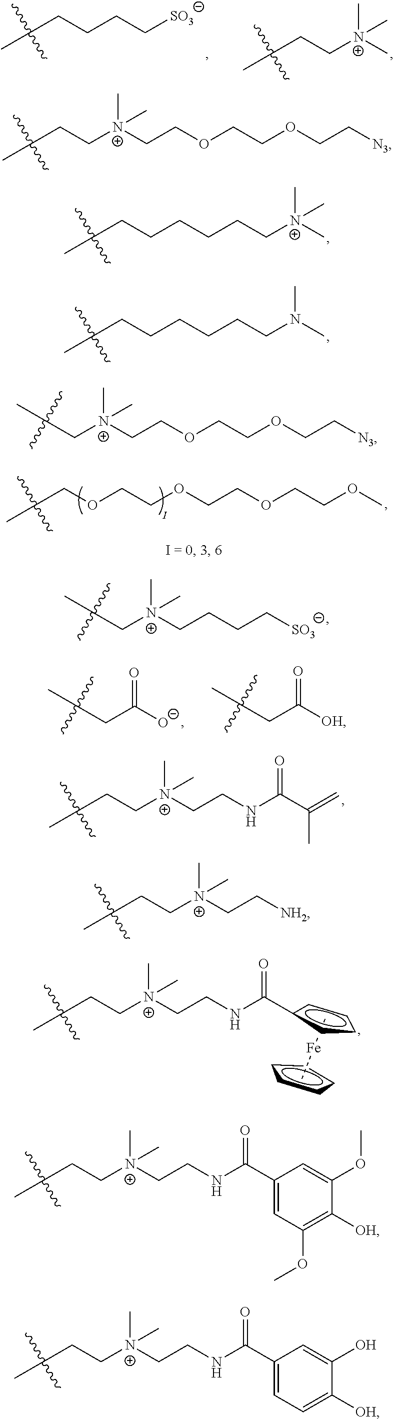 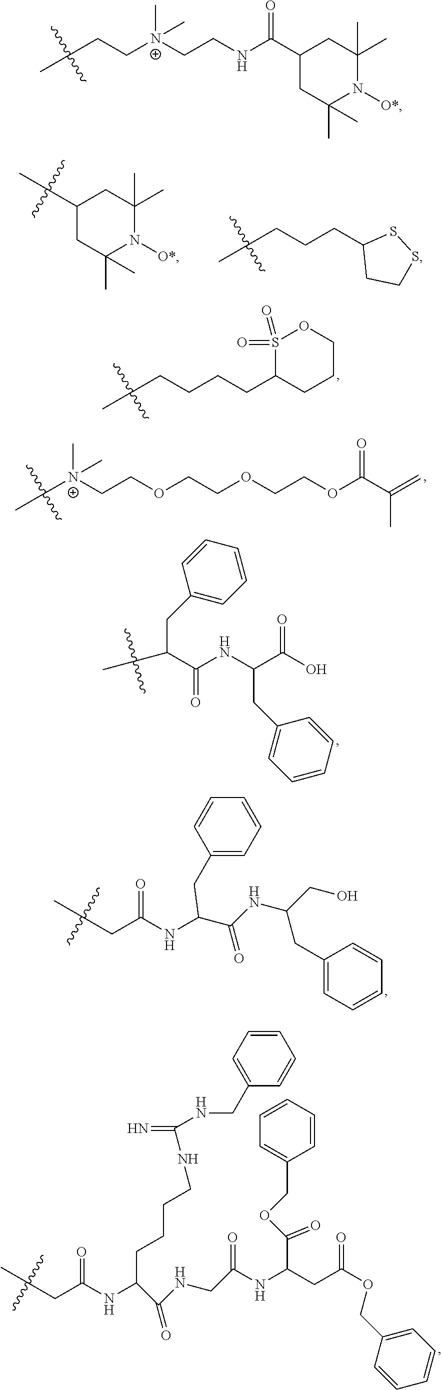 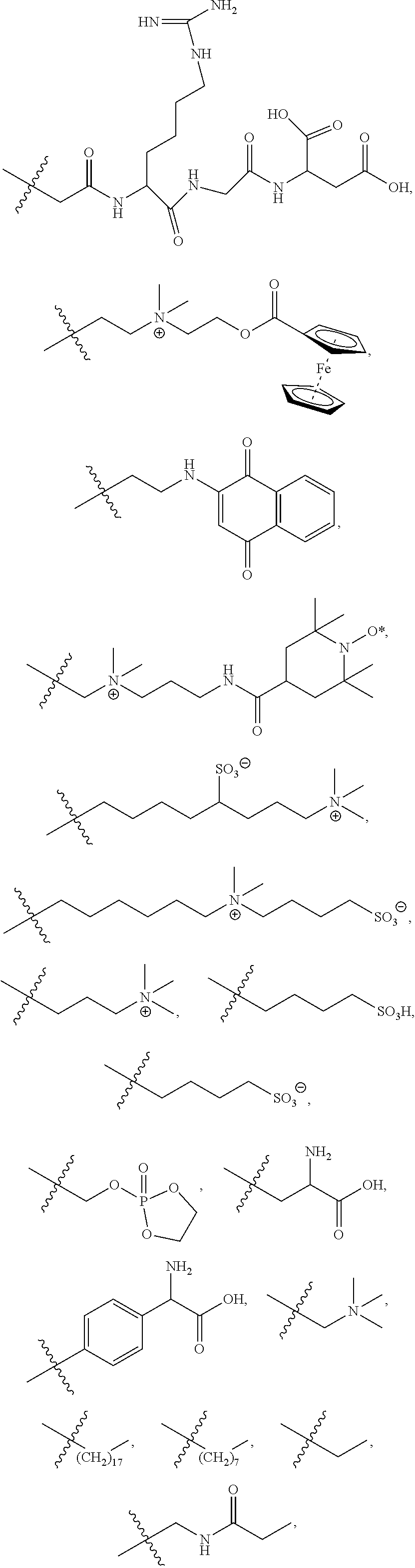 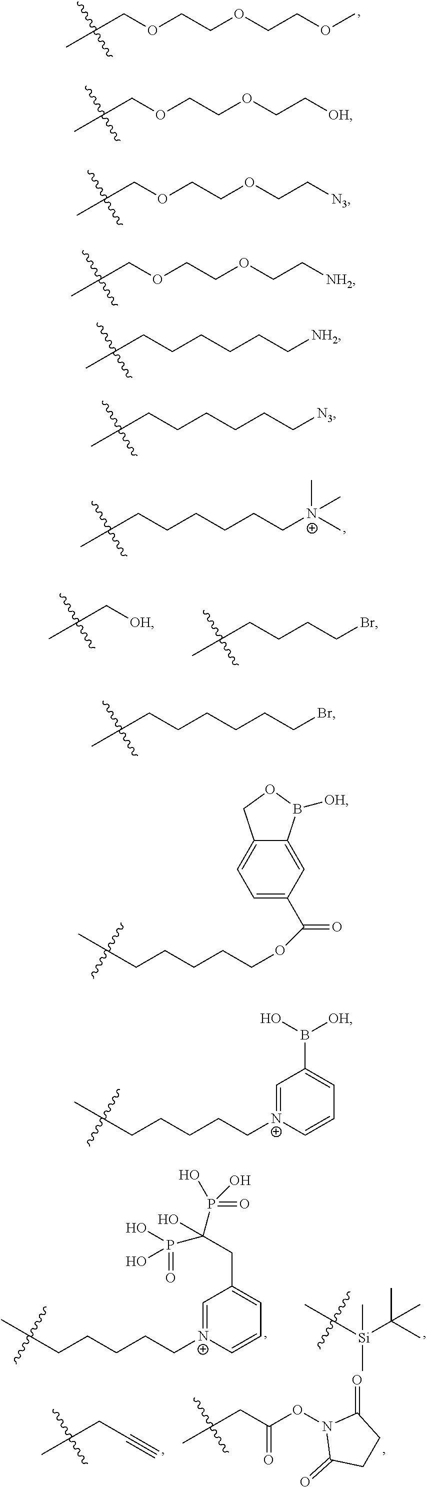 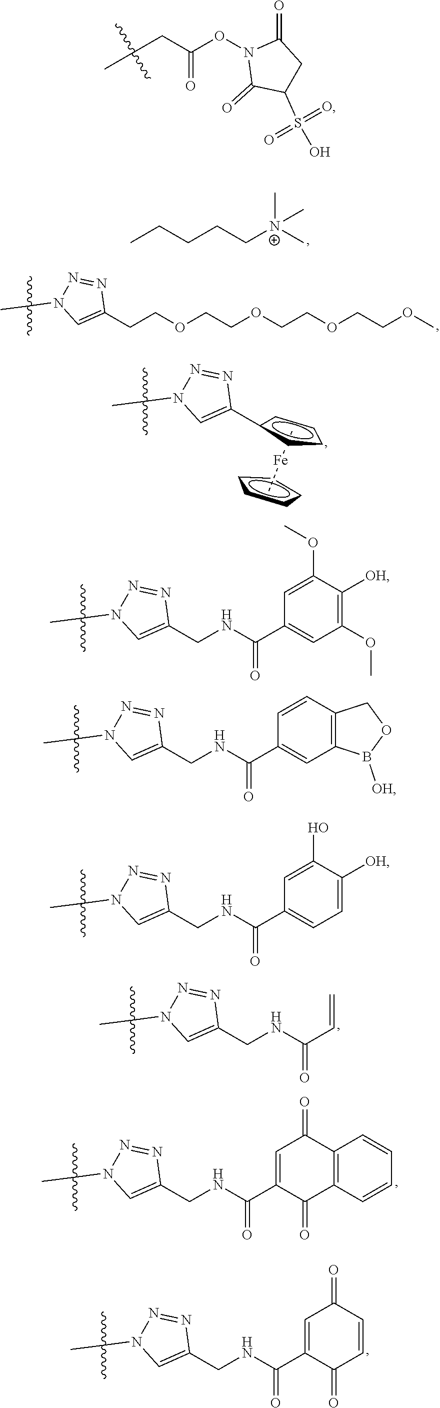
or a pharmaceutically acceptable salt thereof.

7. The composition of claim 1, wherein said composition comprises the polymer represented by the structure 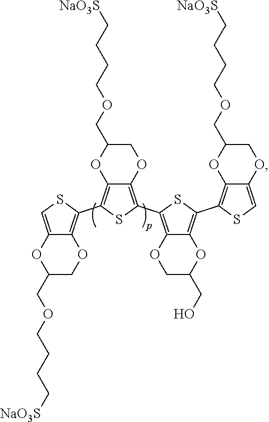
wherein p is 1-12;
and the one or more compound represented by the structures 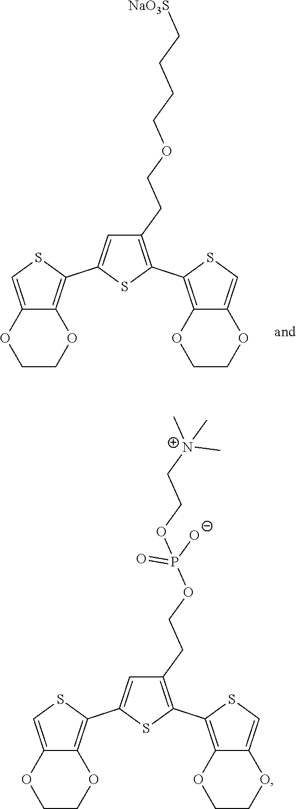
or a pharmaceutically acceptable salt thereof.

8-13. (canceled)

14. A method of treating cancer, a cardiovascular diseases, an infections, a neurodegenerative diseases, or pain, comprising administering a therapeutically effective amount of a composition according to claim 1, or a pharmaceutically acceptable salt thereof, to a patient in need thereof.

15. The method of claim 14, wherein the method is for treating a cancer is-selected from the group consisting of brain cancer, glioblastoma, neuroblastoma, prostate cancer, breast cancer and solid tumors.

16. The composition according to claim 1, wherein the composition is a pharmaceutical composition comprising a pharmaceutically acceptable diluent, carrier and/or excipient.

17. A pharmaceutical composition, comprising a therapeutically effective amount of a composition according to claim 1, or a pharmaceutically acceptable salt thereof, and an additional anticancer agent selected from the group consisting of alkylating agents, antimetabolites, anticancer camptothecin derivatives, plant-derived anticancer agents, antibiotics, enzymes, platinum coordination complexes, tyrosine kinase inhibitors, hormones, hormone antagonists, monoclonal antibodies, interferons, and biological response modifiers.

18. A hydrogel comprising a composition according to claim 1.

19. A kit comprising a composition according to claim 1.

20. The kit of claim 19, wherein the compound of formula (I) is in a first part of the kit, and the one or more compound of formula (II) or formula (III) is in a second part of the kit.

21. The composition of claim 1, wherein said composition comprises the polymer of formula (I) and one or more compounds of formula (II-a):

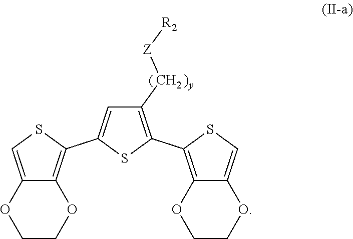

22. The composition of claim 1, wherein R₁ is methyl.

23. The method of claim 14, wherein the method is for treating a neurodegenerative disease selected from the group consisting of brain trauma, spinal cord trauma, trauma to the peripheral nervous system, and motor neuron disease.

24. The method of claim 14, wherein the method is for treating a cardiovascular disease selected from the group consisting of coronary artery diseases (e.g. angina, heart attack), heart failure, hypertensive heart disease, rheumatic heart disease, cardiomyopathy, arrhythmia, congenital heart disease, valvular heart disease, carditis, aortic aneurysms, peripheral artery disease, thromboembolic disease, and venous thrombosis.

25. The method of claim 14, wherein the method is for treating an infection selected from the group consisting of viral infections, bacterial infections, parasitic infections and fungal infections.

26. A method of treating an autoimmune disease, reducing inflammation, or enhancing an immune response against pathogens and tumors, comprising administering a therapeutically effective amount of a composition according to claim 1, or a pharmaceutically acceptable salt thereof, to a patient in need thereof.
